(19) United States
(12) Patent Application Publication
(10) Pub. No.: US 2016/0251408 A1
(43) Pub. Date: Sep. 1, 2016

(54) THROMBIN CLEAVABLE LINKER WITH XTEN AND ITS USES THEREOF

(71) Applicant: BIOGEN MA INC., Cambridge, MA (US)

(72) Inventors: Ekta Seth CHHABRA, Framingham, MA (US); John KULMAN, Belmont, MA (US); Tongyao LIU, Lexington, MA (US)

(73) Assignee: Biogen MA Inc., Cambridge, MA (US)

(21) Appl. No.: 14/894,108

(22) PCT Filed: Jun. 27, 2014
(86) PCT No.: PCT/US2014/044731

§ 371 Date: May 3, 2016

Related U.S. Application Data
(60) Provisional application No. 61/840,872, filed on Jun. 28, 2013.

(51) Int. Cl. C07K 14/755; A61K 38/37

(52) U.S. Cl. CPC C07K 14/755; A61K 38/37; C07K 2319/30; C07K 2319/90; C07K 2319/70; C07K 2319/35; A61K 38/00

(57) ABSTRACT

The present invention provides a chimeric molecule comprising a VWF protein fused to a heterologous moiety via a VWF linker. The invention provides an efficient VWF linker that can be cleaved in the presence of thrombin. The chimeric molecule can further comprise a polypeptide chain comprising a FVIII protein and a second heterologous moiety, wherein the chain comprising the VWF protein and the chain comprising the FVIII protein are associated with each other. The invention also includes nucleotides, vectors, host cells, methods of using the chimeric proteins.

This application claims the benefit of priority of U.S. Provisional Patent Application No. 61/840,872 filed on Jun. 28, 2013. The contents of the above application are incorporated herein by reference in their entirety.

### BACKGROUND OF THE INVENTION

Haemophilia A is a bleeding disorder caused by defects in the gene encoding coagulation factor VIII (FVIII) and affects 1-2 in 10,000 male births. Graw et al., Nat. Rev. Genet. 6(6): 488-501 (2005). Patients affected with hemophilia A can be treated with infusion of purified or recombinantly produced FVIII. All commercially available FVIII products, however, are known to have a half-life of about 8-12 hours, requiring frequent intravenous administration to the patients. See Weiner M. A. and Cairo, M. S., Pediatric Hematology Secrets, Lee, M. T., 12. Disorders of Coagulation, Elsevier Health Sciences, 2001; Lillicrap, D. Thromb. Res. 122 Suppl 4:S2-8 (2008). In addition, a number of approaches have been tried in order to extend the FVIII half-life. For example, the approaches in development to extend the half-life of clotting factors include pegylation, glycopegylation, and conjugation with albumin. See Dumont et al., Blood. 119(13): 3024-3030 (Published online Jan. 13, 2012). Regardless of the protein engineering used, however, the long acting FVIII products currently under development have improved half-lives, but the half-lives are reported to be limited—only to about 1.5 to 2 fold improvement in preclinical animal models. See Id. Consistent results have been demonstrated in humans, for example, rFVIIIFc was reported to improve half-life up to ˜1.7 fold compared with ADVATE® in hemophilia A patients. See Id. Therefore, the half-life increases, despite minor improvements, may indicate the presence of other t_{1/2} limiting factors.

Due to the frequent dosing and inconvenience caused by the dosing schedule, there is still a need to develop FVIII products requiring less frequent administration, i.e., a FVIII product that has a half-life longer than the 1.5 to 2 fold half-life limitation.

### BRIEF SUMMARY OF THE INVENTION

The present invention is directed to a chimeric molecule comprising a Von Willebrand Factor (VWF) protein, a heterologous moiety (H1), an XTEN sequence, and a VWF linker connecting the VWF protein with the heterologous moiety, wherein the linker comprises a polypeptide selected from: (i) an a2 region from Factor VIII (FVIII); (ii) an a1 region from FVIII; (iii) an a3 region from FVIII; (iv) a thrombin cleavage site which comprises X-V-P-R (SEQ ID NO 3) and a PAR1 exosite interaction motif, wherein X is an aliphatic amino acid; or (v) any combination thereof, and wherein the XTEN sequence is connected to the VWF protein, the heterologous moiety (H1), the VWF linker, or any combination thereof. In one embodiment, the XTEN sequence connects the VWF protein with the VWF linker or the VWF linker with the heterologous moiety. In another embodiment, the chimeric molecule further comprises a second polypeptide chain which comprises a FVIII protein, wherein the first polypeptide chain and the second polypeptide chain are associated with each other. In other embodiments, the FVIII protein in the chimeric molecule further comprises an additional XTEN sequence. The additional XTEN sequence can be linked to the N-terminus or the C-terminus of the FVIII protein or inserted between two FVIII amino acids adjacent to each other. In still other embodiments, the second polypeptide chain further comprises a second heterologous moiety (H2).

The instant disclosure also includes a chimeric molecule comprising a first polypeptide chain which comprises a VWF protein, a heterologous moiety (H1), and a VWF linker connecting the VWF protein and the heterologous moiety (H1) and a second polypeptide chain comprising a FVIII protein and an XTEN sequence, wherein the VWF linker in the first polypeptide chain comprises: (i) an a2 region from FVIII; (ii) an a1 region from FVIII; (iii) an a3 region from FVIII; (iv) a thrombin cleavage site which comprises X-V-P-R (SEQ ID NO 3) and a PAR1 exosite interaction motif, wherein X is an aliphatic amino acid; or (v) any combination thereof, and wherein the first polypeptide chain and the second polypeptide chain are associated with each other. In one embodiment, the XTEN sequence is connected to the N-terminus or the C-terminus of the FVIII protein or inserted between two FVIII amino acids adjacent to each other. In another embodiment, the chimeric molecule further comprises an additional XTEN sequence, which is connected to the VWF protein, the heterologous moiety, the VWF linker, or any combination thereof. In other embodiments, the chimeric molecule further comprises a second heterologous moiety (H2). In still other embodiments, the second heterologous moiety is connected to the FVIII protein, the XTEN sequence, or both.

For the chimeric molecules of the present disclosure, the XTEN sequence, either connected to a VWF protein, a VWF linker, a FVIII protein, or any other components in the chimeric molecules, comprises about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, or about 2004 amino acids. In some embodiments, the XTEN polypeptide is selected from AE42, AE72, AE864, AE576, AE288, AE144, AG864, AG576, AG288, or AG144. In other embodiments, the XTEN polypeptide is selected from SEQ ID NO 39; SEQ ID NO 40; SEQ ID NO 47; SEQ ID NO 45; SEQ ID NO 44; SEQ ID NO 41; SEQ ID NO 48; SEQ ID NO 46, SEQ ID NO: 44, or SEQ ID NO: 42.

In other aspects, the additional XTEN sequence in the chimeric molecules comprises about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, or about 2004 amino acids. In some embodiments, the additional XTEN polypeptide is selected from AE42, AE72, AE864, AE576, AE288, AE144, AG864, AG576, AG288, or AG144. In certain embodiments, the additional XTEN polypeptide is selected from SEQ ID NO: 39; SEQ ID NO: 40; SEQ ID NO: 47; SEQ ID NO: 45; SEQ ID NO: 43; SEQ ID NO: 41; SEQ ID NO: 48; SEQ ID NO: 46, SEQ ID NO: 44, or SEQ ID NO: 42.

In one embodiment, the VWF linker useful for connecting a VWF protein and a heterologous moiety in the chimeric molecules comprises an a2 region which comprises an amino acid sequence at least about 80%, about 85%, about 90%, about 95%, or 100% identical to Glu720 to Arg740 corresponding to full-length FVIII, wherein the a2 region is capable of being cleaved by thrombin. In a particular embodiment, the a2 region comprises ISDKNTGDYYEDSYEDISAYLLSKNNAIEPRSFS (SEQ ID NO: 4). In another embodiment, the VWF linker useful for connecting a VWF protein and a heterologous moiety comprises an a1 region which comprises an amino acid sequence at least about 80%, about 85%, about 90%, about 95%, or 100% identical to Met337 to Arg372 corresponding to full-length FVIII, wherein the a1 region is capable of being cleaved by thrombin. In some embodiments, the a1 region comprises ISMKNNEEAEDYDDDLTDSEMDVVRFDDDNSPSFIQIRSV (SEQ ID NO 5).

In other embodiments, the VWF linker useful for connecting a VWF protein and a heterologous moiety comprises an a3 region which comprises an amino acid sequence at least about 80%, about 85%, about 90%, about 95%, or 100% identical to Glu1649 to Arg1689 corresponding to full-length FVIII, wherein the a3 region is capable of being, cleaved by thrombin. In a specific embodiment, the a3 region comprises ISEITRTTLQSDQEEIDYDDTISVEMKKEDFDIYDEDENQSPRSFQ (SEQ ID NO 6).

In still other embodiments, the VWF linker useful for connecting a VWF protein and a heterologous moiety comprises a thrombin cleavage site which comprises X-V-P-R (SEQ ID NO 3) and a PAR1 exosite interaction motif and wherein, the PAR1 exosite interaction motif comprises S-F-L-L-R-N(SEQ ID NO 7). In one embodiment, the PAR1 exosite interaction motif further comprises a sequence selected from P, P-N, P-N-D, P-N-D-K (SEQ ID NO: 8), P-N-D-K-Y (SEQ ID NO: 9), P-N-D-K-Y-E (SEQ ID NO: 10), P-N-D-K-Y-E-P (SEQ ID NO: 11), P-N-D-K-Y-E-P-F (SEQ ID NO 12), P-N-D-K-Y-E-P-F-W (SEQ ID NO: 13), P-N-D-K-Y-E-P-F-W-E (SEQ ID NO: 14), P-N-D-K-Y-E-P-F-W-E-D (SEQ ID NO: 20), P-N-D-K-Y-E-P-F-W-E-D-E (SEQ ID NO: 21), P-N-D-K-Y-E-P-F-W-E-D-E-E (SEQ ID NO 22), P-N-D-K-Y-E-P-F-W-E-D-E-E-S(SEQ ID NO: 23), or any combination thereof. In other embodiment, wherein the aliphatic amino acid is selected from Glycine, Alanine, Valine, Leucine, or Isoleucine. In a particular embodiment, the VWF linker comprises GGLVPRSFLLRNPNDKYEPFWEDEES (SEQ ID NO: 24).

In certain embodiments, thrombin cleaves the VWF linker faster than thrombin, would cleave the thrombin cleavage site if the thrombin cleavage site were substituted for the VWF linker in the chimeric molecule. In other embodiments, thrombin cleaves the VWF linker at least about 10 times, at least about 20 times, at least about 30 times, at least about 40 times, at least about 50 times, at least about 60 times, at least about 70 times, at least about 80 times, at least about 90 times or at least about 100 times faster than thrombin would cleave the thrombin cleavage site if the thrombin cleavage site were substituted for the VWF linker in the chimeric molecule.

In some embodiments, the VWF linker further comprises one or more amino acids having a length of at least about 5, 10, 20, 30, 40, 50, 60, 70, 80, 90, 100, 110, 120, 130, 140, 150, 160, 170, 180, 190, 200, 210, 220, 230, 240, 250, 300, 350, 400, 450, 500, 550, 600, 650, 700, 750, 800, 850, 900, 950, 1000, 1200, 1400, 1600, 1800, or 2000 amino acids. In one example, the one or more amino acids comprise a gly peptide. In another example, the one or more amino acids comprise GlyGly. In other examples, the one or more amino acids comprise a gly/ser peptide. In some examples, the gly/ser peptide has a formula of (Gly₄Ser)n or S(Gly₄Ser)n, wherein n is a positive integer selected from 1, 2, 3, 4, 5, 6, 7, 8, 9, 10, 11, 12, 13, 14, 15, 16, 17, 18, 19, 20, 30, 40, 50, 60, 70, 80, or 100. In certain examples, the (Gly₄Ser)n linker is (Gly₄Ser)₃ (SEQ ID NO: 89) or (Gly₄Ser)₄ (SEQ ID NO: 90).

The VWF protein useful for the chimeric molecule of the invention can comprise the D′ domain and D3 domain of VWF, wherein the D′ domain and D3 domain are capable of binding to a FVIII protein. In one embodiment, the D′ domain of the VWF protein comprises an amino acid sequence at least about 90%, 95%, 96%, 97%, 98%, 99%, or 100% identical to amino acids 764 to 866 of SEQ ID NO: 2. In another embodiment, the D3 domain of the VWF protein comprises an amino acid sequence at least about 90%, 95%, 96%, 97%, 98%, 99%, or 100% identical to amino acids 867 to 1240 of SEQ ID NO 2. In other embodiments, the VWF protein contains at least one amino acid substitution at a residue corresponding to residue 1099, residue 1142, or both residues 1099 and 1142 of SEQ ID NO: 2. In still other embodiments, in the sequence of the VWF protein, an amino acid other than cysteine is substituted for a residue corresponding to residue 1099, residue 1142, or both residues 1099 and 1142 of SEQ ID NO: 2. In yet other embodiments, the sequence of the VWF protein comprises amino acids 764 to 1240 of SEQ ID NO 2. In certain embodiments, the VWF protein further comprises the D1 domain, the D2 domain, or the D1 and D2 domains of VWF. In some embodiments, the VWF protein further comprises a VWF domain selected from the A1 domain, the A2 domain, the A3 domain, the D4 domain, the B1 domain, the B2 domain, the B3 domain, the C1 domain, the C2 domain, the CK domain, one or more fragments thereof, or any combinations thereof. In other embodiments, the VWF protein consists essentially of or consists of (1) the D′ and D3 domains of VWF or fragments thereof; (2) the D1, D′, and D3 domains of VWF or fragments thereof; (3) the D2, D′, and D3 domains of VWF or fragments thereof; (4) the D1, D2, D′, and D3 domains of VWF or fragments thereof; or (5) the D1, D2, D′, D3, and A1 domains of VWF or fragments thereof. In still other embodiments, the VWF protein further comprises a signal peptide of VWF. In yet other embodiments, the VWF protein is pegylated, glycosylated, hesylated, or polysialylated. The term “pegylated” refers to having polyethylene glycol (PEG) on the protein; the term “glycosylated” refers to having glycosylation on the protein; the term “hesylated” refers to having hydroxyethyl starch (HES) on the protein; and the term “polysialylated” refers to having polysialic acids (PSA) on the protein. Examples of PEG, HES, and PSA, are shown elsewhere herein.

In some aspects, the heterologous moiety (H1) fused to the VWF protein via a VWF linker is capable of extending the half-life of the chimeric molecule. In one embodiment, the heterologous moiety (H1) comprises an immunoglobulin constant region or a portion thereof, albumin, albumin-binding moiety, PAS, HAP, transferrin or a fragment thereof, polyethylene glycol (PEG), hydroxyethyl starch (HES), PSA, the C-terminal peptide (CTP) of the β subunit of human chorionic gonadotropin, or any combination thereof. In another embodiment, the heterologous moiety comprises an FcRn binding partner. In other embodiments, the heterologous moiety comprises an Fc region. In other embodiments, the heterologous moiety (H1) comprises a clearance receptor, or fragment thereof, wherein the clearance receptor blocks binding of the FVIII protein to FVIII clearance receptors. In some embodiments, wherein the clearance receptor is a low-density lipoprotein receptor-related protein 1 (LRP1) or FVIII-binding fragment thereof.

In some aspects, the second heterologous moiety fused to the FVIII protein via an optional FVIII linker comprises an immunoglobulin constant region or a portion thereof, albumin, albumin-binding polypeptide, PAS, the C-terminal peptide (CTP) of the β subunit of human chorionic gonadotropin, polyethylene glycol (PEG), hydroxyethyl starch (HES), albumin-binding small molecules, or any combinations thereof. In one embodiment, the second heterologous moiety (H2) is capable of extending the half-life of the FVIII protein. In another embodiment, the second heterologous moiety (H2) comprises a polypeptide, a non-polypeptide moiety, or both. In other embodiment, the second heterologous moiety (H2) comprises an immunoglobulin constant region or a portion thereof. In still other embodiments, the second heterologous moiety comprises an FcRn binding partner. In yet other embodiments, the second heterologous moiety comprises a second Fc region.

In some embodiments, the first heterologous moiety fused to the VWF protein via a VWF linker and the second heterologous moiety fused to the FVIII protein via an optional linker, in which an XTEN sequence is fused to any one of the components, are associated with each other. In one embodiment, the association between the first polypeptide chain and the second polypeptide is a covalent bond. In another embodiment, the association between the first heterologous moiety and the second heterologous moiety is a disulfide bond. In other embodiments, the first heterologous moiety is an FcRn binding partner and the second heterologous moiety is an FcRn binding partner. In still other embodiments, the first heterologous moiety is an Fc region, and the second heterologous moiety is an Fc region.

In certain embodiments, the FVIII protein is linked to the second heterologous moiety by a FVIII linker. In one embodiment, the second linker is a cleavable linker. In another embodiment, the FVIII linker is identical to the VWF linker. In other embodiments, the FVIII linker is different from the VWF linker.

In some aspects, a chimeric molecule of the invention comprises a formula selected from: (a) V-L1-X1-H1:H2-L2-X2-C; (b) V-X1-L1-H1:H2-L2-X2-C; (c) V-L1-X1-H1:H2-X2-L2-C; (d) V-X1-L1-H1:H2-X2-L2-C; (e) V-L1-X1-H1: H2-L2-C(X2); (f) V-X1-L1-H1:H2-L2-C(X2); (g) C-X2-L2-H2:H1-X1-L1-V; (h) C-X2-L2-H2:H1-L1-X1-V; (i) C-L2-X2-H2:H1-L1-X1-V; (j) C-L2-X2-H2:H1-L1-X1-V; (k) C(X2)-L2-H2:H1-X1-L1-V; or (1) C(X2)-L2-H2:H1-L1-X1-V; wherein V is a VWF protein; L1 is a VWF linker; L2 is an optional FVIII linker; H1 is a first heterologous moiety; H2 is a second heterologous moiety; X1 is a XTEN sequence; X2 is an optional XTEN sequence; C is a FVIII protein; C(X2) is a FVIII protein fused to an XTEN sequence, wherein the XTEN sequence is inserted between two FVIII amino acids adjacent to each other; (-) is a peptide bond or one or more amino acids; and (:) is a covalent bond between the H1 and the H2.

In other aspects, a chimeric molecule comprises a formula selected from: (a) V-L1-X1-H1: H2-L2-X2-C; (b) V-X1-L1-H1: H2-L2-X2-C; (c) V-L1-X1-H1: H2-X2-L2-C; (d) V-X1-L1-H1: H2-X2-L2-C; (e) V-L1-X1-H1: H2-L2-C(X2); (f) V-X1-L1-H1: H2-L2-C(X2); (g) C-X2-L2-H2: H1-X1-L1-V; (h) C-X2-L2-H2: H1-L1-X1-V; (i) C-L2-X2-H2:H1-L1-X1-V; (j) C-L2-X2-H2:H1-L1-X1-V; (k) C(X2)-L2-H2:H1-X1-L1-V; or (l) C(X2)-L2-H2:H1-L1-X1-V; wherein V is a VWF protein; L1 is a VWF linker; L2 is an optional FVIII linker; H1 is the first heterologous moiety; H2 is a second heterologous moiety; X1 is an optional XTEN sequence; X2 is an XTEN sequence; C is a FVIII protein; C(X2) is a FVIII protein fused to an XTEN sequence, wherein the XTEN sequence is inserted between two FVIII amino acids adjacent to each other; (-) is a peptide bond or one or more amino acids; and (:) is a covalent bond between the H1 and the H2.

In the chimeric molecules of the invention, the VWF protein can inhibit or prevent binding of endogenous VWF to the FVIII protein.

In certain aspects, the FVIII protein in the chimeric molecules can comprise a third heterologous moiety (H3). The third heterologous moiety (H3) can be an XTEN sequence. In other aspects, the FVIII protein comprises a fourth heterologous moiety (H4). The fourth heterologous moiety (H4) can be an XTEN sequence. In some aspects, the FVIII protein comprises a fifth heterologous moiety (H5). The fifth heterologous moiety can be an XTEN sequence. In other aspects, the FVIII protein comprises the sixth heterologous moiety (H6). The sixth heterologous moiety can be an XTEN sequence. In certain aspects, one or more of the third heterologous moiety (H3), the fourth heterologous moiety (H4), the fifth heterologous moiety (H5), and the sixth heterologous moiety (H6) are capable of extending the half-life of the chimeric molecule. In other aspects, the third heterologous moiety (H3), the fourth heterologous moiety (H4), the fifth heterologous moiety (H5), and the sixth heterologous moiety (H6) are linked to the C terminus or N terminus of FVIII or inserted between two amino acids of the FVIII protein. In still other aspects, one or more of the third heterologous moiety, the fourth heterologous moiety, the fifth heterologous moiety, and the sixth heterologous moiety comprise a length selected from one or more of about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, or about 2004 amino acids. For example, the XTEN sequence of the third heterologous moiety, the fourth heterologous moiety, the fifth heterologous moiety, or the sixth heterologous moiety can be selected from AE42, AE72, AE864, AE576, AE288, AE144, AG864, AG576, AG288, or AG144. More specifically, the XTEN sequence can be selected from SEQ ID NO: 39; SEQ ID NO: 40; SEQ ID NO: 47; SEQ ID NO: 45; SEQ ID NO: 43; SEQ ID NO: 41; SEQ ID NO: 48; SEQ ID NO: 46, SEQ ID NO: 44, or SEQ ID NO: 42.

In certain embodiments, the half-life of the chimeric molecule is extended at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than wild-type FVIII.

The instant disclosure also provides a polynucleotide or a set of polynucleotides encoding a chimeric molecule or a complementary sequence thereof. The polynucleotide or the set of polynucleotides can further comprise a polynucleotide chain, which encodes PC5 or PC7.

Also included is a vector or a set of vectors comprising the polynucleotide or the set of polynucleotides and one or more promoter operably linked to the polynucleotide or the set of polynucleotides. In some embodiments, the vector or the set of vectors can further comprises an additional poly nucleotide chain, encoding PC5 or PC7.

The present invention also includes a host cell comprising the polynucleotide or the set of the polynucleotides or the vector or the set of vectors. In one embodiment, the host cell is a mammalian cell. In another embodiment, the host cell is selected from a HEK293 cell, CHO cell, or BHK cell.

In some aspects, the invention includes a pharmaceutical composition comprising a chimeric molecule disclosed herein, the polynucleotide or the set of polynucleotides encoding the chimeric molecule, the vector or the set of vectors comprising the polynucleotide or the set of polynucleotides, or the host cell disclosed herein, and a pharmaceutically acceptable carrier. In one embodiment, the chimeric molecule in the composition has extended half-life compared to wild type FVIII protein. In another embodiment, wherein the half-life of the chimeric molecule in the composition is extended at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than, wild type FVIII.

Also included is a method of reducing a frequency or degree of a bleeding episode in a subject in need thereof comprising administering an effective amount of a chimeric molecule disclosed herein, the polynucleotide or the set of polynucleotides encoding the chimeric molecule, the vector or the set of vectors disclosed herein, the host cell disclosed herein, or the composition disclosed herein. The invention also includes a method of preventing an occurrence of a bleeding episode in a subject in need thereof comprising administering an effective amount of a chimeric molecule disclosed herein, the polynucleotide or the set of polynucleotides encoding the chimeric molecule, the vector or the set of vectors disclosed herein, the host cell disclosed herein, or the composition disclosed herein. In one embodiment, the bleeding episode is from a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combinations thereof. In another embodiment, a chimeric molecule disclosed herein, the polynucleotide or the set of polynucleotides encoding the chimeric molecule, the vector or the set of vectors disclosed herein, the host cell disclosed herein, or the composition disclosed herein can be administered by a route selected from topical administration, intraocular administration, parenteral administration, intrathecal administration, subdural administration, oral administration, or any combinations thereof.

The instant disclosure also includes a method of making a chimeric molecule, comprising transfecting one or more host cell with a polynucleotide disclosed herein or a vector disclosed herein and expressing the chimeric molecule in the host cell. The method further comprises isolating the chimeric molecule. In some embodiments, the FVIII activity of the chimeric molecule can be measured by aPTT assay or ROTEM assay.

### BRIEF DESCRIPTION OF THE DRAWINGS/FIGURES

FIG. 1 shows an exemplary diagram of a chimeric molecule (FVIII-XTEN/VWF heterodimer) comprising two polypeptide chains, the first chain comprising a VWF protein (e.g., a D′ domain and a D3 domain of VWF) fused to an Fc region via a thrombin cleavable VWF linker and the second chain comprising a FVIII protein fused to a second Fc region via a FVIII linker. The FVIII protein comprises one or more XTENs in various domains of FVIII.

FIG. 2 shows various VWF constructs, each construct comprising a D′ domain and a D3 domain fused to an Fc region via a thrombin cleavable VWF linker except control (i.e., VWF-052). VWF-031 comprises a linker of 48 amino acids comprising a thrombin cleavage site of L-V-P-R (SEQ ID NO 25). VWF-034 comprises an XTEN sequence having 288 amino acids and a linker of 35 amino acids comprising a thrombin cleavage site of L-V-P-R (SEQ ID NO 25). VWF-035 comprises a linker of 73 amino acids comprising a thrombin cleavage site of L-V-P-R (SEQ ID NO: 25). VWF-036 comprises a linker of 98 amino acids comprising a thrombin cleavage site of L-V-P-R (SEQ ID NO 25). VWF-039 comprises a VWF linker of 26 amino acids comprising a thrombin cleavage site of L-V-P-R (SEQ ID NO 25) and a PAR1 exosite interaction motif VWF-051 comprises a linker of 54 amino acids, comprising a thrombin cleavage site of A-L-R-P-R-V-V (SEQ ID NO 26). VWF-052 comprises a linker of 48 amino acids without any thrombin cleavage site (control). VWF-054 comprises a VWF linker of 40 amino acids comprising an a1 region from FVIII. VWF-055 comprises a VWF linker of 34 amino acids comprising an a2 region from FVIII. VWF-056 comprises a VWF linker of 46 amino acids comprising an a3 region from FVIII.

FIG. 3A shows the rate of thrombin-mediated cleavage in units of resonance units per second (RU/s) as a function of capture density in units of RU for VWF-Fc fusion constructs, VWF-031, VWF-034, VWF-036, VWF-039, VWF-051, and VWF-052. FIG. 3B shows the rate of thrombin-mediated cleavage in units of resonance units per second (RU/s) as a function of capture density in units of RU for VWF-Fc fusion constructs, i.e., VWF-031, VWF-034, VWF-036, VWF-051, and VWF-052. In these experiments, each VWF-Fc fusion construct was captured at various densities and subsequently exposed to a fixed concentration of human alpha-thrombin. The slope of each curve in FIG. 3A and FIG. 3B directly reflect the susceptibility to thrombin cleavage for each construct.

FIG. 4A shows the rate of thrombin-mediated cleavage in units of resonance units per second (RU/s) as a function of capture density in units of RU for VWF-Fc fusion constructs, i.e., VWF-054, VWF-055, and VWF-056. FIG. 4B shows the rate of thrombin-mediated cleavage in units of resonance units per second (RU/s) as a function of capture density in units of RU for VWF-Fc fusion constructs, i.e., VWF-031. VWF-039, VWF-054, VWF-055, and VWF-056. In these experiments, each VWF-Fc fusion construct was captured at various densities and subsequently exposed to a fixed concentration of human alpha-thrombin. The slopes of each curve in FIG. 4A and FIG. 4B directly reflect the susceptibility to thrombin cleavage for each construct.

FIG. 5 shows the results of a linear regression analysis to determine the susceptibility of various VWF-Fc constructs, VWF-031, VWF-034, VWF-036, VWF-039, VWF-051, VWF-052, VWF-054, VWF-055, and VWF-056, to thrombin-mediated cleavage. Values are expressed in units of inverse seconds and reflect the slopes of the curves presented in FIG. 3 and FIG. 4. The relative susceptibility of two different constructs is derived from the quotient of their respective slopes. Slope_{VWF-039}/slope_{VWF-031} is 71, indicating that VWF-Fc fusion construct VWF-039 is 71-fold more susceptible to thrombin-mediated cleavage than is VWF-031. Slope_{VWF-055}/slope_{VWF-031} is 65, and slope_{VWF-051}/slope_{VWF-031} is 1.8.

FIG. 6 shows clotting time of various chimeric molecules in a HemA patient measured by whole blood ROTEM assay. FVII155/VWF-031 comprises two polypeptide chains, the first chain comprising BDD FVIII fused to an Fc region and the second chain comprising a D′ domain and a D3 domain of VWF fused to an Fc region via a minimal thrombin cleavage site (i.e., L-V-P-R (SEQ ID NO: 25)). FVII155/VWF-039 comprises two polypeptide chains, the first chain comprising BDD FVIII fused to an Fc region and the second chain comprising a D′ domain and a D3 domain of VWF fused to an Fc region via a VWF linker comprising L-V-P-R (SEQ ID NO: 25) and a PAR1 exosite interaction motif. FVII155/VWF-055 comprises two polypeptide chains, the first chain comprising BDD FVIII fused to an Fc region and the second chain comprising a D′ domain and a D3 domain of VWF fused to an Fc region via a VWF linker comprising an a2 region from FVIII.

FIG. 7 shows a diagram of representative FWIII-VWF heterodimer and FVIII169, FVIII286, VWF057, tWF0599, and VWF062 constructs. For example, FVIII169 construct comprises a B domain deleted FVIII protein with R1648A substitution fused to an Fc region, wherein an XTEN sequence (e.g., AE288) is inserted at amino acid 745 corresponding to mature full length FVIII (A1-a1-A2-a2-288XTEN-a3-A3-C1-C2-Fc). FVIII286 construct comprises a B domain deleted FVIII protein with R1648 substitution fused to an Fc region, wherein an XTEN sequence (e.g., AE288) is inserted at amino acid 745 corresponding to mature full length FVIII, with additional a2 region in between FVIII and Fc (A1-a1-A2-a2-288XTEN-a3-A3-C1-C2-a2-Fc). VWF057 is a VWF-Fc fusion construct that comprises D′D3 domain of the VWF protein (with two amino acid substitutions in D′D3 domain, i.e., C336A and C379A) linked to the Fc region via a VWF linker, which comprises LVPR thrombin site (“LVPR”) and GS linker (“GS”), wherein an XTEN sequence (i.e., 144XTEN) is inserted between D′D3 domain and the VWF linker (D′D3-144XTEN-GS+LVPR-Fc). VWF059 is a VWF-Fc fusion construct that comprises D′D3 domain of the VWF protein (with two amino acid substitutions in D′D3 domain, i.e., C336A and C379A) linked to the Fc region via an acidic region 2 (a2) region as a VWF linker, wherein an XTEN sequence is inserted between D′D3 domain and the VWF linker. VWF062 is a VWF-Fc fusion construct that comprises D′D3 domain of the VWF protein (with two amino acid substitutions in D′D3 domain, i.e., C336A and C379A) linked to the Fc region, wherein an XTEN sequence is inserted between D′D3 domain and the Fc region (D′D3-144XTEN-Fc).

FIG. 8 shows, acute efficacy of FVIII-XTEN-Fc/D′D3-Linker-Fc heterodimers (i.e., FVIII169/VWF034, FVIII169/VWF059, and FVIII169/VWF057), compared to B domain deleted FVIII (“SQ BDD FVIII” or “BDD-rFVIII”) or vehicle control in HemA, mice tail clip model. BDD-rFVIII is shown as circle while FVIII169/VWF034 is shown as square, FVIII169/VWF059 is shown as triangle, FVIII169/VWF057 is shown as hollow circle, and vehicle is shown as inverted triangle. VWF034 is a VWF-Fc fusion construct that comprises a D′ domain and a D3 domain of VWF fused an Fc region via a VWF linker, which comprises LVPR, wherein an XTEN sequence (i.e., 288XTEN) is inserted between D′D3 domain and the VWF linker (D′D3-288XTEN-LVPR-Fc). The construct details of FVIII169, VWF059, and VWF057 are shown elsewhere herein. The median blood loss (uL) of mice after dosing of 75 IU/kg of the construct in each treatment groups are indicated by the horizontal lines.

### DETAILED DESCRIPTION OF THE INVENTION

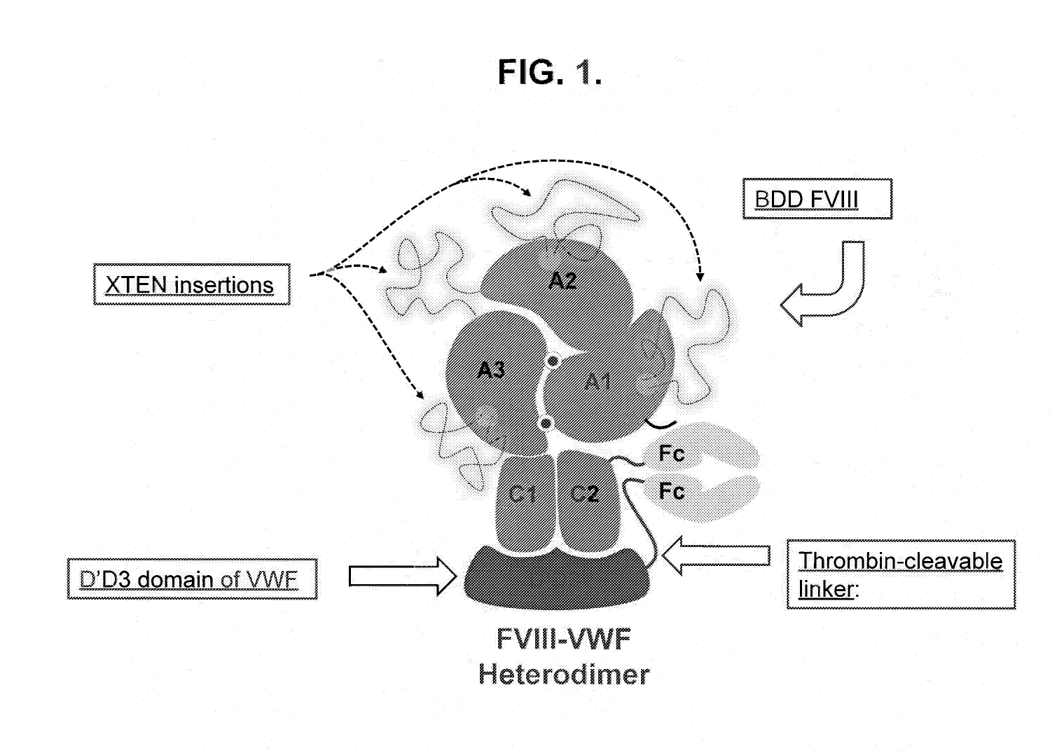

The present invention is directed to a chimeric molecule comprising an XTEN sequence and a thrombin cleavable linker connecting a VWF protein or a FVIII protein with a heterologous moiety, e.g., a half-life extending moiety. The invention also provides a chimeric molecule comprising two polypeptide chains, the first chain comprising a VWF protein fused to a heterologous moiety, and a second chain comprising a FVIII protein and a second heterologous moiety, wherein the chimeric molecule comprises an XTEN sequence in the first or second polypeptide chains and wherein either the VWF protein or the FVIII protein (or both) is fused to the heterologous moiety via a VWF linker or a FVIII linker (or both). The thrombin cleavable linker (VWF linker or FVIII linker) can be cleaved efficiently by thrombin at the site of injury where thrombin is readily available. Exemplary chimeric molecules are illustrated in the instant description and figures. In some embodiments, the invention pertains to chimeric molecules having the structures set forth, for example, in FIGS. 1 to 7. In other embodiments, the invention pertains to polynucleotide encoding chimeric molecule constructs disclosed herein.

In order to provide a clear understanding of the specification and claims, the following definitions are provided below.

### I. DEFINITIONS

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The term “polynucleotide” or “nucleotide” is intended to encompass a singular nucleic acid as well as plural nucleic acids, and refers to an isolated nucleic acid molecule or construct, e.g., messenger RNA (mRNA) or plasmid DNA (pDNA). In certain embodiments, a polynucleotide comprises a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a Factor VIII polypeptide contained in a vector is considered isolated for the purposes of the present invention. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present invention. Isolated polynucleotides or nucleic acids according to the present invention further include such molecules produced synthetically. In addition, a polynucleotide or a nucleic acid can include regulatory elements such as promoters, enhancers, ribosome binding sites, or transcription termination signals.

As used herein, a “coding region” or “coding sequence” is a portion of polynucleotide which consists of codons translatable into amino acids. Although a “stop codon” (TAG, TGA, or TAA) is typically not translated into an amino acid, it may be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′terminus, encoding the carboxyl terminus of the resulting polypeptide. Two or more coding regions of the present invention can be present in a single polynucleotide construct, e.g., on a single vector, or in separate polynucleotide constructs, e.g., on separate (different) vectors. It follows, then, that a single vector can contain just a single coding region, or comprise two or more coding regions, e.g., a single vector can separately encode a first polypeptide chain and a second polypeptide chain of a chimeric molecule as described below. In addition, a vector, polynucleotide, or nucleic acid of the invention can encode heterologous coding regions, either fused or unfused to a nucleic acid encoding a chimeric molecule of the invention. Heterologous coding regions include without limitation specialized elements or motifs, such as a secretory signal peptide or a heterologous functional domain.

Certain proteins secreted by mammalian cells are associated with a secretory signal peptide which is cleaved from the mature protein once export of the growing protein chain across the rough endoplasmic reticulum has been initiated. Those of ordinary skill in the art are aware that signal peptides are generally fused to the N-terminus of the polypeptide, and are cleaved from the complete or “full-length” polypeptide to produce a secreted or “mature” form of the polypeptide. In certain embodiments, a native signal peptide, e.g., a FVIII signal, peptide or a VWF signal peptide is used, or a functional derivative of that sequence that retains the ability to direct the secretion of the polypeptide that is operably associated with it. Alternatively, a heterologous mammalian signal peptide, e.g., a human tissue plasminogen activator (TPA) or mouse β-glucuronidase signal peptide, or a functional derivative thereof, can be used.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions may include promoters, translation leader sequences, introns, polyadenylation recognition sequences, RNA processing sites, effector binding sites and stem-loop structures. If a coding region is intended for expression in a eukaryotic cell, a polyadenylation signal and transcription termination sequence will usually be located 3′ to the coding sequence.

A polynucleotide which encodes a gene product, e.g., a polypeptide, can include a promoter and/or other transcription or translation control elements operably associated with one or more coding regions. In an operable association a coding region for a gene product, e.g., a polypeptide, is associated with, one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other transcription control elements, besides a promoter, for example enhancers, operators, repressors, and transcription termination signals, can also be operably associated with a coding region to direct gene product expression.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation, initiation and termination codons, and elements derived from picornaviruses (particularly an internal ribosome entry site, or IRES, also referred to as a CITE sequence).

The term “expression” as used herein refers to a process by which, a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

A “vector” refers to any vehicle for the cloning of and/or transfer of a nucleic acid into a host cell. A vector may be a replicon to which another nucleic acid segment may be attached so as to bring about the replication of the attached segment. A “replicon” refers to any genetic element (e.g., plasmid, phage, cosmid, chromosome, virus) that functions as an autonomous unit of replication in vivo, i.e., capable of replication under its own control. The term “vector” includes both viral and nonviral vehicles, for introducing the nucleic acid into a cell in vitro, ex vivo or in vivo. A large number of vectors are known and used in the art including, for example, plasmids, modified eukaryotic viruses, or modified bacterial viruses. Insertion of a polynucleotide into a suitable vector can be accomplished by ligating the appropriate polynucleotide fragments into a chosen vector that has complementary cohesive termini.

Vectors may be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and, used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), -galactosidase (LacZ), -glucuronidase (Gus), and the like. Selectable markers may also be considered to be reporters.

The term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

Eukaryotic viral vectors that can be used include, but are not limited to, adenovirus vectors, retrovirus vectors, adeno-associated virus vectors, poxvirus, e.g., vaccinia virus vectors, baculovirus vectors, or herpesvirus vectors. Non-viral vectors include plasmids, liposomes, electrically charged lipids (cytofectins), DNA-protein complexes, and biopolymers.

A “cloning vector” refers to a “replicon,” which is a unit length of a nucleic acid that replicates sequentially and which comprises an origin of replication, such as a plasmid, phage or cosmid, to which another nucleic acid segment may be attached so as to bring about the replication of the attached segment. Certain cloning vectors are capable of replication in one cell type, e.g., bacteria and expression in another, e.g., eukaryotic cells. Cloning vectors typically comprise one or more sequences that can be used for selection of cells comprising the vector and/or one or more multiple cloning sites for insertion of nucleic acid sequences of interest.

The term “expression vector” refers to a vehicle designed to enable the expression of an inserted nucleic acid sequence following insertion into a host cell. The inserted nucleic acid sequence is placed in operable association with regulatory regions as described above.

Vectors are introduced into host cells by methods well known in the art, e.g., transfection, electroporation, microinjection, transduction, cell fusion, DEAE dextran, calcium phosphate precipitation, lipofection (lysosome fusion), use of a gene gun, or a DNA vector transporter.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within, the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced recombinant technology, but is not necessarily translated from a designated nucleic acid sequence. It can be generated in any manner, including by chemical synthesis.

An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

Also included in the present invention are fragments or variants of polypeptides, and any combination thereof. The term “fragment” or “variant” when referring to polypeptide binding domains or binding molecules of the present invention include any polypeptides which retain at least some of the properties (e.g., FcRn binding affinity for an FcRn binding domain or Fc variant, coagulation activity for an FVIII variant, or FVIII binding activity for the VWF protein) of the reference polypeptide. Fragments of polypeptides include proteolytic fragments, as well as deletion fragments, in, addition to specific antibody fragments discussed elsewhere herein, but do not include the naturally occurring full-length polypeptide (or mature polypeptide). Variants of polypeptide binding domains or binding molecules of the present invention include fragments as descried above, and also polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “VWF fragment” or “VWF fragments” used herein means any VWF fragments that interact with FVIII and retain at least one or more properties that are normally provided to FVIII by full-length VWF, e.g., preventing premature activation to FVIIIa, preventing premature proteolysis, preventing association with phospholipid membranes that could lead to premature clearance, preventing binding to FVIII clearance receptors that can bind naked FVIII but not VWF-bound FVIII, and/or stabilizing the FVIII heavy chain and light chain interactions. In a particular embodiment, the “VWF fragment” as used herein comprises a D′ domain and a D3 domain of the VWF protein, but does not include the A1 domain, the A2 domain, the A3 domain, the D4 domain, the B1 domain, the B2 domain, the B3 domain, the C1 domain, the C2 domain, and the CK domain of the VWF protein.

The term “half-life limiting factor” or “FVIII half-life limiting, factor” as used herein indicates a factor that prevents the half-life of a FVIII protein from being longer than 1.5 fold or 2 fold compared to wild-type FVIII (e.g., ADVATE® or REFACTO®). For example, full length or mature VWF can act as a FVIII half-life limiting factor by inducing the FVIII and VWF complex to be cleared from system by one or more VWF clearance pathways. In one example, endogenous VWF is a FVIII half-life limiting factor. In another example, a full-length recombinant VWF molecule non-covalently bound to a FVIII protein is a FVIII-half-life limiting factor.

The term “endogenous VWF” as used herein indicates VWF molecules naturally present in plasma. The endogenous VWF molecule can be multimer, but can be a monomer or a dimer. Endogenous VWF in plasma binds to FVIII and forms a non-covalent complex with FVIII.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

As known in the art, “sequence identity” between two polypeptides is determined by comparing the amino acid sequence of one polypeptide to the sequence of a second polypeptide. When discussed herein, whether any particular polypeptide is at least about 50%, 60%, 70%, 75%, 80%, 85%, 90%, 95%, 99%, or 100% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full-length of the reference polypeptide sequence and that gaps in homology of up to 5% of the total number of amino acids in the reference sequence are allowed.

As used herein, an “amino acid corresponding to” or an “equivalent amino acid” in a VWF sequence or a FVIII protein sequence is identified by alignment to maximize the identity or similarity between a first VWF or FVIII sequence and a second VWF or FVIII sequence. The number used to identify an equivalent amino acid in a second VWF or FVIII sequence is based on the number used to identify the corresponding amino acid in the first VWF or FVIII sequence.

A “fusion” or “chimeric” molecule comprises a first amino acid sequence linked to a second amino acid sequence with which it is not naturally linked in nature. The amino acid sequences which normally exist in separate proteins can be brought together in the fusion polypeptide, or the amino acid sequences which normally exist in the same protein can be placed in a new arrangement in the fusion polypeptide, e.g., fusion of a Factor VIII domain of the invention with an immunoglobulin Fc domain. A fusion protein is created, for example, by chemical synthesis, or by creating and translating a polynucleotide in which the peptide regions are encoded in the desired relationship. A chimeric protein can further comprises a second amino acid sequence associated with the first amino acid sequence by a covalent, non-peptide bond or a non-covalent bond.

As used herein, the term “half-life” refers to a biological half-life of a particular polypeptide in vivo. Half-life may be represented by the time required for half the quantity administered to a subject to be cleared from the circulation and/or other tissues in the animal. When a clearance curve of a given polypeptide is constructed as a function of time, the curve is usually biphasic with a rapid α-phase and longer β-phase. The α-phase typically represents an equilibration of the administered polypeptide between the intra- and extra-vascular space and is, in part, determined by the size of the polypeptide. The β-phase typically represents the catabolism of the polypeptide in the intravascular space. In some embodiments, chimeric molecule of the invention are monophasic, and thus do not have an alpha phase, but just the single beta phase. Therefore, in certain embodiments, the term half-life as used herein refers to the half-life of the polypeptide in the β-phase. The typical β phase half-life of a human antibody in humans is 21 days.

The term “heterologous” as applied to a polynucleotide or a polypeptide, means that the polynucleotide or polypeptide is derived from a distinct entity from that of the entity to which it is being compared. Therefore, a heterologous polypeptide linked to a VWF protein means a polypeptide chain that is linked to a VWF protein and is not a naturally occurring part of the VWF protein. For instance, a heterologous polynucleotide or antigen can be derived from a different species, different cell type of an individual, or the same or different type of cell of distinct individuals.

The term “linked,” “fused,” or “connected” as used herein refers to a first amino acid sequence or nucleotide sequence joined to a second amino acid sequence or nucleotide sequence (e.g., via a peptide bond or a phosphodiester bond, respectively). The term “covalently linked” or “covalent linkage” refers to a covalent bond, e.g., a disulfide bond, a peptide bond, or one or more amino acids, e.g., a linker, between the two moieties that are linked together. The first amino acid or nucleotide sequence can be directly joined to the second amino acid or nucleotide sequence or alternatively an intervening sequence can join the first sequence to the second sequence. The term “linked,” “fused,” or “connected” means not only a fusion of a first amino acid sequence to a second amino acid sequence at the C-terminus or the N-terminus, but also includes insertion of the whole first amino acid sequence (or the second amino acid sequence) into any two amino acids in the second amino acid sequence (or the first amino acid sequence, respectively). In one embodiment, the first amino acid sequence can be joined to a second amino acid sequence by a peptide bond or a linker. The first nucleotide sequence can be joined to a second nucleotide sequence by a phosphodiester bond or a linker. The linker can be a peptide or a polypeptide (for polypeptide chains) or a nucleotide or a nucleotide chain (for nucleotide chains) or any chemical moiety (for both polypeptide and polynucleotide chains). The covalent linkage is sometimes indicated as (-) or hyphen.

As used herein the term “associated with” refers to a covalent or non-covalent bond formed between a first amino acid chain and a second amino acid chain. In one embodiment, the term “associated with” means a covalent, non-peptide bond or a non-covalent bond. In some embodiments this association is indicated by a colon, i.e., (:). In another embodiment, it means a covalent bond except a peptide bond. In other embodiments, the term “covalently associated” as used herein means an association between two moieties by a covalent bond, e.g., a disulfide bond, a peptide bond, or one or more amino acids (e.g., a linker). For example, the amino acid cysteine comprises a thiol group that can form a disulfide bond or bridge with a thiol group on a second cysteine residue. In most naturally occurring IgG molecules, the CH1 and CL regions are associated by a disulfide bond and the two heavy chains are associated by two disulfide bonds at positions corresponding to 239 and 242 using the Kabat numbering system (position 226 or 229, EU numbering system). Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, an hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity.

As used herein, the term “cleavage site” or “enzymatic cleavage site” refers to a site recognized by an enzyme. In one embodiment, a polypeptide has an enzymatic cleavage site cleaved by an enzyme that is activated during the clotting cascade, such that cleavage of such sites occurs at the site of clot formation. In another embodiment, a FVIII linker connecting a FVIII protein and a second heterologous moiety can comprise a cleavage site. Exemplary such sites include e.g., those recognized by thrombin, Factor XIa or Factor Xa. Exemplary FXIa cleavage sites include, e.g, TQSFNDFTR (SEQ ID NO: 27) and SVSQTSKLTR (SEQ ID NO: 28). Exemplary thrombin cleavage sites include, e.g, DFLAEGGGVR (SEQ ID NO: 29), TTKIKPR (SEQ ID NO: 30), LVPRG (SEQ ID NO: 31) and ALRPR (amino acids 1 to 5 of SEQ ID NO: 26). Other enzymatic cleavage sites are known in the art. A cleavage site that can be cleaved by thrombin is referred to herein as “thrombin cleavage site.”

As used herein, the term “processing site” or “intracellular processing site” refers to a type of enzymatic cleavage site in a polypeptide which is the target for enzymes that function after translation of the polypeptide. In one embodiment, such enzymes function during transport from the Golgi lumen to the trans-Golgi compartment. Intracellular processing enzymes cleave polypeptides prior to secretion of the protein from the cell. Examples of such processing sites include, e.g., those targeted by the PACE/furin (where PACE is an acronym for Paired basic Amino acid Cleaving Enzyme) family of endopeptidases. These enzymes are localized to the Golgi membrane and cleave proteins on the carboxy terminal side of the sequence motif Arg-[any residue]-(Lys or Arg)-Arg. As used herein the “furin” family of enzymes includes, e.g., PCSK1 (also known as PC1/Pc3), PCSK2 (also known as PC2), PCSK3 (also known as furin or PACE), PCSK4 (also known as PC4), PCSK5 (also known as PC5 or PC6), PCSK6 (also known as PACE4), or PCSK7 (also known as PC7/LPC, PC8, or SPC7). Other processing sites are known in the art. The term “processable linker” referred to herein means a linker comprising an intracellular processing site.

The term “furin” refers to the enzymes corresponding to EC No. 3.4.21.75. Furin is subtilisin-like proprotein convertase, which is also known as PACE (Paired basic Amino acid Cleaving Enzyme). Furin deletes sections of inactive precursor proteins to convert them into biologically active proteins. During its intracellular transport, pro-peptide is cleaved from mature VWF molecule by a furin enzyme in the Golgi.

In constructs that include more than one processing or cleavage site, it will be understood that such sites may be the same or different.

Hemostatic disorder, as used herein, means a genetically inherited or acquired condition characterized by a tendency to hemorrhage, either spontaneously or as a result of trauma, due to an impaired ability or inability to form a fibrin clot. Examples of such disorders include the hemophilias. The three main forms are hemophilia A (factor VIII deficiency), hemophilia B (factor IX deficiency or “Christmas disease”) and hemophilia C (factor XI deficiency, mild bleeding tendency). Other hemostatic disorders include, e.g., von Willebrand disease, Factor XI deficiency (PTA deficiency), Factor XII deficiency, deficiencies or structural abnormalities in fibrinogen, prothrombin, Factor V, Factor VII, Factor X or factor XIII, Bernard-Soulier syndrome, which is a defect or deficiency in GPIb. GPIb, the receptor for VWF, can be defective and lead to lack of primary clot formation (primary hemostasis) and increased bleeding tendency), and thrombasthenia of Glanzman and Naegeli (Glanzmann thrombasthenia). In liver failure (acute and chronic forms), there is insufficient production of coagulation factors by the liver; this may increase bleeding risk.

The chimeric molecules of the invention can be used prophylactically. As used herein the term “prophylactic treatment” refers to the administration of a molecule prior to a bleeding episode. In one embodiment, the subject in need of a general hemostatic agent is undergoing, or is about to undergo, surgery. The chimeric protein of the invention can be administered prior to or after surgery as a prophylactic. The chimeric protein of the invention can be administered during or after surgery to control an acute bleeding episode. The surgery can include, but is not limited to, liver transplantation, liver resection, dental procedures, or stem cell transplantation.

The chimeric molecule of the invention is also used for on-demand (also referred to as “episodic”) treatment. The term “on-demand treatment” or “episodic treatment” refers to the administration of a chimeric molecule in response to symptoms of a bleeding episode or before an activity that may cause bleeding. In one aspect, the on-demand (episodic) treatment can be given to a subject when bleeding starts, such as after an injury, or when bleeding is expected, such as before surgery. In another aspect, the on-demand treatment can be given prior to activities that increase the risk of bleeding, such as contact sports.

As used herein the term “acute bleeding” refers to a bleeding episode regardless of the underlying cause. For example, a subject may have trauma, uremia, a hereditary bleeding disorder (e.g., factor VII deficiency) a platelet disorder, or resistance owing to the development of antibodies to clotting factors.

Treat, treatment, treating, as used herein refers to, e.g., the reduction in severity of a disease or condition; the reduction in the duration of a disease course; the amelioration of one or more symptoms associated with a disease or condition; the provision of beneficial effects to a subject with a disease or condition, without necessarily curing the disease or condition, or the prophylaxis of one or more symptoms associated with a disease or condition. In one embodiment, the term “treating” or “treatment” means maintaining a FVIII trough level at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, or 20 IU/dL in a subject by administering a chimeric molecule of the invention. In another embodiment, treating or treatment means maintaining a FVIII trough level between about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, or 20% of the FVIII activity in a non-hemophiliac subject. The minimum trough level required for treatment can be measured by one or more known methods and can be adjusted (increased or decreased) for each person.

### II. CHIMERIC MOLECULES

Chimeric molecules of the invention are designed to improve release of a VWF protein or a FVIII protein from another moiety that the VWF protein or FVIII protein is fused to. The invention provides a thrombin cleavable linker that can be cleaved fast and efficient at the site of injury. In one aspect of the invention, a chimeric molecule can comprise a von Willebrand Factor (VWF) protein, a heterologous moiety (H1), an XTEN sequence, and a VWF linker connecting the VWF protein with the heterologous moiety, wherein the VWF linker comprises a polypeptide selected from: (i) an a2 region from Factor VIII (FVIII); (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof, and wherein the XTEN sequence is connected to the VWF protein, the heterologous moiety (H1), the VWF linker, or any combination thereof. In another aspect of the invention, a chimeric molecule can comprise a first polypeptide chain which comprises a VWF protein, a heterologous moiety (H1), and a VWF linker connecting the VWF protein and the heterologous moiety (H1) and a second polypeptide chain comprising a FVIII protein and an XTEN sequence, wherein the VWF linker in the first polypeptide chain comprises: (i) an a2 region from FVIII; (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof, and wherein the first polypeptide chain and the second polypeptide chain are associated with each other.

In other aspects of the inventin, a chimeric molecule comprises a polypeptide chain comprising a FVIII protein fused to a hererologous moiety via a FVIII linker, wherein the FVIII linker comprises: (i) an a2 region from FVIII; (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof.

II.A. Chimeric Molecules with VWF, XTEN, VWF Linker

The present invention provides a chimeric molecule comprising a VWF protein fused to an XTEN sequence via a VWF linker, wherein the VWF linker comprises a polypeptide selected from: (i) an a2 region from FVIII; (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof.

In one embodiment, a chimeric molecule comprises a VWF protein, a heterologous moiety (H1), an XTEN sequence, and a VWF linker connecting the VWF protein with the heterologous moiety, wherein the XTEN sequence is located between the VWF protein and the VWF linker and wherein the VWF linker comprises a polypeptide selected from: (i) an a2 region from Factor VIII (FVIII); (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof. In another embodiment, a chimeric molecule comprises a VWF protein, a heterologous moiety (H1), an XTEN sequence, and a VWF linker connecting the VWF protein with the heterologous moiety, wherein the XTEN sequence is located between the VWF linker and the heterologous moiety and wherein the VWF linker comprises a polypeptide selected from: (i) an a2 region from FVIII; (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof.

In other embodiments, the chimeric molecule further comprises a polypeptide chain, which comprises a FVIII protein, wherein the first chain comprising the VWF protein and the second chain comprising the FVIII protein are associated with each other. In one example, the association can be a covalent, e.g., disulfide bond, association. In still other embodiments, the polypeptide chain comprising a FVIII protein further comprises an additional XTEN sequence. The additional XTEN sequence can be linked to the N-terminus or the C-terminus of the FVIII protein or inserted between two FVIII amino acids adjacent to each other. In yet other embodiments, the chain comprising a FVIII protein further comprises a second heterologous moiety (H2). In some embodiments, the FVIII protein is fused to the second heterologous moiety via a FVIII linker. In certain embodiments, the FVIII linker is identical to the VWF linker connecting the VWF protein and the heterologous moiety. In other embodiments, the FVIII Linker is different from the VWF linker connecting the VWF protein and the heterologous moiety.

In certain embodiments, a chimeric molecule comprises a formula selected from: (i) V-L1-X1-H1:H2-L2-X2-C; (ii) V-X1-L1-H1:H2-L2-X2-C; (iii) V-L1-X1-H1:H2-X2-L2-C; (iv) V-X1-L-H1:H2-X2-L2-C; (v) V-L1-X1-H1:H2-L2-C(X2); (vi) V-X1-L1-H1:H2-L2-C(X2); (vii) C-X2-L2-H2:H1-X1-L1-V; (viii) C-X2-L2-H2:H1-L1-X1-V; (ix) C-L2-X2-H2:H1-L1-X1-V; (x) C-L2-X2-H2:H1-L1-X1-V; (xi) C(X2)-L2-H2:H1-X1-L1-V; or (xii) C(X2)-L2-H2:H1-L1-X1-V; wherein V is a VWF protein; L1 is a VWF linker; L2 is an optional FVIII linker; H1 is a first heterologous moiety; H2 is a second heterologous moiety; X1 is a XTEN sequence; X2 is an optional XTEN sequence; C is a FVIII protein; C(X2) is a FVIII protein fused to an XTEN sequence, wherein the XTEN sequence is inserted between two FVIII amino acids adjacent to each other; (-) is a peptide bond or one or more amino acids; and (:) is a covalent bond between the H1 and the H2.

In some embodiments, the FVIII protein in the chimeric molecule comprises a third heterologous moiety (H3), which can be an XTEN sequence. In other embodiments, the FVIII protein of the chimeric molecule comprises a fourth heterologous moiety (H4), which can be an XTEN sequence. In still other embodiments, the FVIII protein of the chimeric molecule comprises a fifth heterologous moiety (H5), which can be an XTEN sequence. In yet other embodiments, the FVIII protein of the chimeric molecule comprises the sixth heterologous moiety (H6), which can be an XTEN sequence. In certain embodiments, one or more of the third heterologous moiety (H3), the fourth heterologous moiety (H4), the fifth heterologous moiety (H5), and the sixth heterologous moiety (H6) are capable of extending the half-life of the chimeric molecule. In some embodiments, the third heterologous moiety (H3), the fourth heterologous moiety (H4), the fifth heterologous moiety (H5), and the sixth heterologous moiety (H6) are linked to the C-terminus or N-terminus of FVIII or inserted between two amino acids of the FVIII protein.

II.B. Chimeric Molecules with FVIII, XTEN, VWF Protein, VWF Linker

The instant invention also provides a chimeric molecule comprising a first polypeptide chain which comprises a VWF protein, a heterologous moiety (H1), and a VWF linker connecting the VWF protein and the heterologous moiety (H1) and a second polypeptide chain comprising a FVIII protein and an XTEN sequence, wherein the VWF linker in the first polypeptide chain comprises: (i) an a2 region from FVIII; (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof, and wherein the first polypeptide chain and the second polypeptide chain are associated with each other. In one embodiment, wherein the XTEN sequence is connected to the N-terminus or the C-terminus of the FVIII protein or inserted between two FVIII amino acids adjacent to each other. In another embodiment, the chimeric molecule further comprises an additional XTEN sequence, which is connected to the VWF protein, the heterologous moiety, the VWF linker, or any combination thereof. In other embodiments, the chimeric molecule further comprises a second heterologous moiety (H2). In other embodiments, the second heterologous moiety of the chimeric molecule is connected to the FVIII protein, the XTEN sequence, or both. In still other embodiments, the second heterologous moiety is connected to the FVIII protein or the XTEN sequence via a FVIII linker. In yet other embodiments, the FVIII linker is identical to the VWF linker. In some embodiments, the FVIII linker is different from the VWF linker.

In certain embodiments, a chimeric molecule comprises a formula selected from: (i) V-L1-X1-H1:H2-L2-X2-C; (ii) V-X1-L1-H1:H2-L2-X2-C; (iii) V-L1-X1-H1:H2-X2-L2-C; (iv) V-X1-L1-H1:H2-X2-L2-C; (v) V-L1-X-H1:H2-L2-C(X2); (vi) V-X1-L1-H1:H2-L2-C(X2); (vii) C-X2-L2-H2:H1-X1-L1-V; (viii) C-X2-L2-H2:H1-L1-X1-V; (ix) C-L2-X2-H2:H1-L1-X1-V; (x) C-L2-X2-H2:H1-L1-X1-V; (xi) C(X2)-L2-H2:H1-X1-L1-V; or (xii) C(X2)-L2-H2:H1-L1-X1-V; wherein V is a VWF protein; L is a VWF linker; L2 is an optional FVIII linker; H1 is a first heterologous moiety; H2 is a second heterologous moiety; X1 is an optional XTEN sequence; X2 is an XTEN sequence; C is a FVIII protein; C(X2) is a FVIII protein fused to an XTEN sequence, wherein the XTEN sequence is inserted between two FVIII amino acids adjacent to each other; (-) is a peptide bond or one or more amino acids; and (:) is a covalent bond between the H1 and the H2. In one embodiment, the VWF linker and the FVIII linker can be the same. In another embodiment, the VWF linker and the FVIII linker are different.

In certain embodiments, the FVIII protein of the chimeric molecule comprises a third heterologous moiety (H3), which can be an XTEN sequence. In other embodiments, the FVIII protein of the chimeric molecule comprises a fourth heterologous moiety (H4), which is an XTEN sequence. In still other embodiments, the FVIII protein of the chimeric molecule comprises a fifth heterologous moiety (H5), which can be an XTEN sequence In yet other embodiments, the FVIII protein comprises the sixth heterologous moiety (H6), which can be an XTEN sequence. In certain embodiments, one or more of the third heterologous moiety (H3), the fourth heterologous moiety (H4), the fifth heterologous moiety (H5), and the sixth heterologous moiety (H6) are capable of extending the half-life of the chimeric molecule. In some embodiments, the third heterologous moiety (H3), the fourth heterologous moiety (H4), the fifth heterologous moiety (H5), and/or the sixth heterologous moiety (H6) are linked to the C terminus or N terminus of FVIII or inserted between two amino acids of the FVIII protein.

II.C. Chimeric Molecules with FVIII, XTEN, and FVIII Linker

A chimeric molecule of the invention can comprise a FVIII protein, an XTEN sequence, and a heterologous moiety fused by a FVIII linker, which comprises (i) an a2 region from FVIII; (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof. In certain embodiments, a chimeric molecule comprises two polypeptide chains, the first chain comprising a FVIII protein fused to a first Fc region via a FVIII linker, and a second chain comprising a VWF protein (e.g., a D′ domain and a D3 domain of VWF) fused to an Fc region, wherein the FVIII linker in the first polypeptide chain comprises: (i) an a2 region from FVIII; (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof, and wherein the first polypeptide chain and the second polypeptide chain are associated with each other and wherein an XTEN sequence is linked to the first polypeptide (e.g., N terminus or C terminus of the FVIII protein, the linker, or the first Fc region or within the FVIII protein), the second polypeptide (e.g., N terminus or C terminus of the VWF protein or the Fc region or within the FVIII protein), or both. In a specific embodiment the linker in the first polypeptide chain comprises an a2 region from FVIII.

In certain embodiments, a chimeric molecule comprises a formula selected from: (i) V-L2-X2-H2: H1-L1-X1-C; (ii) V-X2-L2-H2: H1-L1-X1-C; (iii) V-L2-X2-H2: H1-X1-L1-C; (iv) V-X2-L2-H2: H1-X1-L1-C; (v) V-L2-X2-H2: H1-L1-C(X1); (vi) V-X2-L2-H2: H1-L1-C(X1); (vii) C-X1-L1-H1: H2-X2-L2-V; (viii) C-X1-L1-H1: H2-L2-X2-V; (ix) C-L1-X1-H1:H2-L2-X2-V; (x) C-L1-X1-H1:H2-L2-X2-V; (xi) C(X1)-L1-H1:H2-X2-L2-V; or (xii) C(X1)-L1-H:H2-L2-X2-V, wherein V is a VWF protein; L1 is a FVIII linker; L2 is an optional VWF linker; H1 is a first heterologous moiety; H2 is a second heterologous moiety; X1 is an optional XTEN sequence; X2 is an optional XTEN sequence; C is a FVIII protein; C(X1) is a FVIII protein fused to an XTEN sequence, wherein the XTEN sequence is inserted between two FVIII amino acids adjacent to each other; (-) is a peptide bond or one or more amino acids; and (:) is a covalent bond between the H1 and the H2 and wherein at least one XTEN sequence is present in the chimeric molecule. In one embodiment, the VWF linker and the FVIII linker are the same. In another embodiment, the VWF linker and the FVIII linker are different.

### II.D. Components of Chimeric Molecules

II.C.1. VWF Linker or FVIII Linker

The VWF linker or FVIII linker useful for a chimeric molecule of the invention is a thrombin cleavable linker fusing a VWF protein with a heterologous moiety or a FVIII protein with a heterologous moiety. In one embodiment, the VWF linker or FVIII linker comprises an a1 region of FVIII. In another embodiment, the VWF linker or FVIII linker comprises an a2 region of FVIII. In other embodiments, the VWF linker or FVIII linker comprises an a3 region of FVIII. In yet other embodiments, the VWF linker or FVIII linker comprises a thrombin cleavage site which comprises X-V-P-R (SEQ ID NO: 3) and a PAR1 exosite interaction motif, wherein X is an aliphatic amino acid.

In one embodiment, the VWF linker or FVIII linker comprises an a1 region which comprises an amino acid sequence at least about 80%, about 85%, about 90%, about 95%, about 96%, about 97%, about 98%, about 99%, or 100% identical to Met337 to Arg372 corresponding to full-length mature FVIII, wherein the a1 region is capable of being cleaved by thrombin. In another embodiment, the VWF linker or FVIII linker comprises an a1 region which comprises an amino acid sequence at least about 80%, about 85%, about 90%, about 95%, about 96%, about 97%, about 98%, about 99%, or 100% identical to amino acids 337 to 374 corresponding to full-length mature FVIII, wherein the a1 region is capable of being cleaved by thrombin. In other embodiments, the VWF linker or FVIII linker further comprises additional amino acids, e.g., one, two, three, four, five, ten, or more. In a particular embodiment, the VWF linker or FVIII linker comprises ISMKNNEEAEDYDDDLTDSEMDVVRFDDDNSPSFIQIRSV (SEQ ID NO: 5).

In some embodiments, the VWF linker or FVIII linker comprises an a2 region which comprises an amino acid sequence at least about 80%, about 85%, about 90%, about 95%, about 96%, about 97%, about 98%, about 99%, or 100% identical to Glu720 to Arg740 corresponding to full-length mature FVIII, wherein the a2 region is capable of being cleaved by thrombin. In other embodiments, the VWF linker or FVIII linker comprises an a2 region which comprises an amino acid sequence at least about 80%, about 85%, about 90%, about 95%, about 96%, about 97%, about 98%, about 99%, or 100% identical to amino acids 712 to 743 corresponding to full-length mature FVIII. In still other embodiments, the VWF linker or FVIII linker further comprises additional amino acids, e.g., one, two, three, four, five, ten, or more. In a particular embodiment, the VWF linker comprises ISDKNTGDYYEDSYEDISAYLLSKNNAIEPRSFS (SEQ ID NO: 4)

In certain embodiments, the VWF linker or FVIII linker comprises an a3 region which comprises an amino acid sequence at least about 80%, about 85%, about 90%, about 95%, about 96%, about 97%, about 98%, about 99%, or 100% identical to Glu1649 to Arg1689 corresponding to full-length mature FVIII, wherein the a3 region is capable of being cleaved by thrombin. In some embodiments, the VWF linker or FVIII linker comprises an a3 region which comprises an amino acid sequence at least about 80%, about 85%, about 90%, about 95%, about 96%, about 97%, about 98%, about 99%, or 100% identical to amino acids 1649 to 1692 corresponding to full-length mature FVIII, wherein the a3 region is capable of being cleaved by thrombin. In other embodiments, the VWF linker or FVIII linker further comprises additional amino acids, e.g., one, two, three, four, five, ten, or more. In a specific embodiment, a VWF linker or FVIII linker comprises ISEITRTTLQSDQEEIDYDDTISVEMKKEDFDIYDEDENQSPRSFQ (SEQ ID NO: 6)

In other embodiments, the VWF linker or FVIII linker comprises a thrombin cleavage site comprising X-V-P-R (SEQ ID NO: 3) and a PAR1 exosite interaction motif and wherein the PAR1 exosite interaction motif comprises S-F-L-L-R-N(SEQ ID NO: 7). In some embodiments, the PAR1 exosite interaction motif further comprises an amino acid sequence selected from P, P-N, P-N-D, P-N-D-K (SEQ ID NO: 8), P-N-D-K-Y (SEQ ID NO: 9), P-N-D-K-Y-E (SEQ ID NO: 10), P-N-D-K-Y-E-P (SEQ ID NO: 11), P-N-D-K-Y-E-P-F (SEQ ID NO: 12), P-N-D-K-Y-E-P-F-W (SEQ ID NO: 13), P-N-D-K-Y-E-P-F-W-E (SEQ ID NO: 14), P-N-D-K-Y-E-P-F-W-E-D (SEQ ID NO: 20), P-N-D-K-Y-E-P-F-W-E-D-E (SEQ ID NO: 21), P-N-D-K-Y-E-P-F-W-E-D-E-E (SEQ ID NO: 22), P-N-D-K-Y-E-P-F-W-E-D-E-E-S(SEQ ID NO: 23), or any combination thereof. In other embodiments, the aliphatic amino acid for the thrombin cleavage site comprising X-V-P-R is selected from Glycine, Alanine, Valine, Leucine, or Isoleucine. In a specific embodiment, the thrombin cleavage site comprises L-V-P-R. In some embodiments, thrombin cleaves the VWF linker or FVIII linker faster than thrombin would cleave the thrombin cleavage site (e.g., L-V-P-R) if the thrombin cleavage site (L-V-P-R) were substituted for the VWF linker or FVIII linker, respectively (i.e., without the PAR1 exosite interaction motif). In some embodiments, thrombin cleaves the VWF linker or FVIII linker at least about 10 times, at least about 20 times, at least about 30 times, at least about 40 times, at least about 50 times, at least about 60 times, at least about 70 times, at least about 80 times, at least about 90 times or at least about 100 times faster than thrombin would cleave the thrombin cleavage site (e.g., L-V-P-R) if the thrombin cleavage site (e.g., L-V-P-R) were substituted for the VWF linker or FVIII linker.

In some embodiments, a VWF linker or FVIII linker comprising (i) an a1 region, (ii) an a2 region, (iii) an a3 region or (iv) a thrombin cleavage site X-V-P-R and a PAR1 exosite interaction motif further comprises one or more amino acids having a length of at least about 2, 5, 10, 20, 30, 40, 50, 60, 70, 80, 90, 100, 110, 120, 130, 140, 150, 160, 170, 180, 190, 200, 210, 220, 230, 240, 250, 300, 350, 400, 450, 500, 550, 600, 650, 700, 750, 800, 850, 900, 950, 1000, 1200, 1400, 1600, 1800, or 2000 amino acids. In one embodiment, the one or more amino acids comprise a gly peptide. In another embodiment, the one or more amino acids comprise GlyGly. In other embodiments, the one or more amino acids comprise IleSer. In still other embodiments, the one or more amino acids comprise a gly/ser peptide. In yet other embodiments, the one or more amino acids comprise a gly/ser peptide having a formula of (Gly₄Ser)n or S(Gly₄Ser)n, wherein n is a positive integer selected from 1, 2, 3, 4, 5, 6, 7, 8, 9, 10, 11, 12, 13, 14, 15, 16, 17, 18, 19, 20, 30, 40, 50, 60, 70, 80, or 100. In some embodiments, the one or more amino acids comprise (Gly₄Ser)₃ (SEQ ID NO: 89) or (Gly₄Ser)₄ (SEQ ID NO: 90).

II.C.2. VWF Protein

VWF (also known as F8VWF) is a large multimeric glycoprotein present in blood plasma and produced constitutively in endothelium (in the Weibel-Palade bodies), megakaryocytes (a-granules of platelets), and subendothelian connective tissue. The basic VWF monomer is a 2813 amino acid protein. Every monomer contains a number of specific domains with a specific function, the D′/D3 domain (which binds to Factor VIII), the A1 domain (which binds to platelet GPIb-receptor, heparin, and/or possibly collagen), the A3 domain (which binds to collagen), the C1 domain (in which the RGD domain binds to platelet integrin αIIbβ3 when this is activated), and the “cysteine knot” domain at the C-terminal end of the protein (which VWF shares with platelet-derived growth factor (PDGF), transforming growth factor-β (TGFβ) and β-human chorionic gonadotropin (βHCG)).

The term “VWF protein” as used herein includes, but is not limited to, full-length VWF protein or functional VWF fragments comprising a D′ domain and a D3 domain, which are capable of inhibiting binding of endogenous VWF to FVIII. In one embodiment, a VWF protein binds to FVIII. In another embodiment, the VWF protein blocks the VWF binding site on FVIII, thereby inhibiting interaction of FVIII with endogenous VWF. In other embodiments, a VWF protein is not cleared by a VWF clearance pathway. The VWF proteins include derivatives, variants, mutants, or analogues that retain these activities of VWF.

The 2813 monomer amino acid sequence for human VWF is reported as Accession Number_NP_000543.2_in Genbank. The nucleotide sequence encoding the human VWF is reported as Accession Number_NM_000552.3_in Genbank. The nucleotide sequence of human VWF is designated as SEQ ID NO: 1. SEQ ID NO: 2 is the amino acid sequence encoded by SEQ ID NO: 1. Each domain of VWF is listed in Table 1.

TABLE 1
VWF Sequences

| VWF domains | | |
|---|---|---|
| | Amino acid Sequence | |
| VWF Signal Peptide | 1 | MIPARFAGVL LALALILPGT LC 22 |
| (Amino acids 1 to 22 | | |
| of SEQ ID NO: 2) | | |
| VWF D1D2 region | 23 | AEGTRGRS STARCSLFGS |
| (Amino acids 23 to | | DFVNTFDGSM |
| 763 of SEQ ID NO: 2) | 51 | YSFAGYCSYL LAGGCQKRSF SIIGDFQNGK RVSLSVYLGE |
| | | FFDIHLFVNG |
| | 101 | TVTQGDQRVS MPYASKGLYL ETEAGYYKLS GEAYGFVARI |
| | | DGSGNFQVLL |
| | 151 | SDRYFNKTCG LCGNFNIFAE DDFMTQEGTL TSDPYDFANS |
| | | WALSSGEQWC |
| | 201 | ERASPPSSSC NISSGEMQKG LWEQCQLLKS TSVFARCHPL |
| | | VDPEPFVALC |
| | 251 | EKTLCECAGG LECACPALLE YARTCAQEGM VLYGWTDHSA |
| | | CSPVCPAGME |
| | 301 | YRQCVSPCAR TCQSLHINEM CQERCVDGCS CPEGQLLDEG |
| | | LCVESTECPC |
| | 351 | VHSGKRYPPG TSLSRDCNTC ICRNSQWICS NEECPGECLV |
| | | TGQSHFKSFD |
| | 401 | NRYFTFSGIC QYLLARDCQD HSFSIVIETV QCADDRDAVC |
| | | TRSVTVRLPG |
| | 451 | LHNSLVKLKH GAGVAMDGQD IQLPLLKGDL RIQHTVTASV |
| | | RLSYGEDLQM |
| | 501 | DWDGRGRLLV KLSPVYAGKT CGLCGNYNGN QGDDFLTPSG |
| | | LAEPRVEDFG |
| | 551 | NAWKLHGDCQ DLQKQHSDPC ALNPRMTRFS EEACAVLTSP |
| | | TFEACHRAVS |
| | 601 | PLPYLRNCRY DVCSCSDGRE CLCGALASYA AACAGRGVRV |
| | | AWREPGRCEL |
| | 651 | NCPKGQVYLQ CGTPCNLTCR SLSYPDEECN EACLEGCFCP |
| | | PGLYMDERGD |
| | 701 | CVPKAQCPCY YDGEIFQPED IFSDHHTMCY CEDGFMHCTM |
| | | SGVPGELLPD |
| | 751 | AVLSSPLSHR SKR 763 |
| VWF D′ Domain | 764 | SLSCRPP MVKLVCPADN LRAEGLECTK |
| | | TCQNYDLECM |
| | 801 | SMGCVSGCLC PPGMVRHENR CVALERCPCF HQGKEYAPGE |
| | | TVKIGCNTCV |
| | 851 | CRDRKWNCTD HVCDAT 866 |
| VWF D3 Domain | 867 | CSTI GMAHYLTFDG LKYLFPGECQ |
| | | YVLVQDYCGS |
| | 901 | NPGTFRILVG NKGCSHPSVK CKKRVTILVE GGEIELFDGE |
| | | VNVKRPMKDE |
| | 951 | THFEVVESGR YIILLLGKAL SVVWDRHLSI SVVLKQTYQE |
| | | KVCGLCGNFD |
| | 1001 | GIQNNDLTSS NLQVEEDPVD FGNSWKVSSQ CADTRKVPLD |
| | | SSPATCHNNI |
| | 1051 | MKQTMVDSSC RILTSDVFQD CNKLVDPEPY LDVCIYDTCS |
| | | CESIGDCACF |
| | 1101 | CDTIAAYAHV CAQHGKVVTW RTATLCPQSC EERNLRENGY |
| | | ECEWRYNSCA |
| | 1151 | PACQVTCQHP EPLACPVQCV EGCHAHCPPG KILDELLQTC |
| | | VDPEDCPVCE |
| | 1201 | VAGRRFASGK KVTLNPSDPE HCQICHCDVV NLTCEACQEP |
| | 1240 | |
| VWF A1 Domain | 1241 | GGLVVPPTDA |
| | 1251 | PVSPTTLYVE DISEPPLHDF YCSRLLDLVF LLDGSSRLSE |
| | | AEFEVLKAFV |
| | 1301 | VDMMERLRIS QKWVRVAVVE YHDGSHAYIG LKDRKRPSEL |
| | | RRIASQVKYA |
| | 1351 | GSQVASTSEV LKYTLFQIFS KIDRPEASRI ALLLMASQEP |
| | | QRMSRNFVRY |
| | 1401 | VQGLKKKKVI VIPVGIGPHA NLKQIRLIEK QAPENKAFVL |
| | | SSVDELEQQR |
| | 1451 | DEIVSYLCDL APEAPPPTLP PDMAQVTVG 1479 |
| | 1480 | P GLLGVSTLGP KRNSMVLDVA |
| | 1501 | FVLEGSDKIG EADFNRSKEF MEEVIQRMDV GQDSIHVTVL |
| | | QYSYMVTVEY |
| | 1551 | PFSEAQSKGD ILQRVREIRY QGGNRTNTGL ALRYLSDHSF |
| | | LVSQGDREQA 1600 |
| | 1601 | PNLVYMVTGN PASDEIKRLP GDIQVVPIGV GPNANVQELE |
| | | RIGWPNAPIL |
| | 1651 | IQDFETLPRE APDLVLQRCC SGEGLQIPTL SPAPDCSQPL |
| | | DVILLLDGSS |
| | 1701 | SFPASYFDEM KSFAKAFISK ANIGPRLTQV SVLQYGSITT |
| | | IDVPWNVVPE |
| | 1751 | KAHLLSLVDV MQREGGPSQI GDALGFAVRY LTSEMHGARP |
| | | GASKAVVILV |
| | 1801 | TDVSVDSVDA AADAARSNRV TVFPIGIGDR YDAAQLRILA |
| | | GPAGDSNVVK |
| | 1851 | LQRIEDLPTM VTLGNSFLHK LCSGFVRICM DEDGNEKRPG |
| | | DVWTLPDQCH |
| | 1901 | TVTCQPDGQT LLKSHRVNCD RGLRPSCPNS QSPVKVEETC |
| | | GCRWTCPCVC |
| | 1951 | TGSSTRHIVT FDGQNFKLTG SCSYVLFQNK EQDLEVILHN |
| | | GACSPGARQG |
| | 2001 | CMKSIEVKHS ALSVEXHSDM EVTVNGRLVS VPYVGGNMEV |
| | | NVYGAIMHEV |
| | 2051 | RFNHLGHIFT FTPQNNEFQL QLSPKTFASK TYGLCGICDE |
| | | NGANDFMLRD |
| | 2101 | GTVTTDWKTL VQEWTVQRPG QTCQPILEEQ CLVPDSSHCQ |
| | | VLLLPLFAEC |
| | 2151 | HKVLAPATFY AICQQDSCHQ EQVCEVIASY AHLCRTNGVC |
| | | VDWRTPDFCA |
| | 2201 | MSCPPSLVYN HCEHGCPRHC DGNVSSCGDH PSEGCFCPPD |
| | | KVMLEGSCVP |
| | 2251 | EEACTQCIGE DGVQHQFLEA WVPDHQPCQI CTCLSGRKVN |
| | | CTTQPCPTAK |
| | 2301 | APTCGLCEVA RLRQNADQCC PEYECVCDPV SCDLPPVPHC |
| | | ERGLQPTLTN |
| | 2351 | PGECRPNFTC ACRKEECKRV SPPSCPPHRL PTLRKTQCCD |
| | | EYECACNCVN |
| | 2401 | STVSCPLGYL ASTATNLCGC TTTTCLPDKV CVHRSTIYPV |
| | | GQFWEEGCDV |
| | 2451 | CTCTDMEDAV MGLRVAQCSQ KPCEDSCRSG FTYVLHEGEC |
| | | CGRCLPSACE |
| | 2501 | VVTGSPRGDS QSSWKSVGSQ WASPENPCLI NECVRVKEEV |
| | | FIQQRNVSCP |
| | 2551 | QLEVPVCPSG FQLSCKTSAC CPSCRCERME ACMLNGTVIG |
| | | PGKTVMIDVC |
| | 2601 | TTCRCMVQVG VISGFKLECR KTTCNPCPLG YKEENNTGEC |
| | | CGRCLPTACT |
| | 2651 | IQLRGGQIMT LKRDETLQDG CDTHFCKVNE RGEYFWEKRV |
| | | TGCPPFDEHK |
| | 2701 | CLAEGGKIMK IPGTCCDTCE EPECNDITAR LQYVKVGSCK |
| | | SEVEVDIHYC |
| | 2751 | QGKCASKAMY SIDINDVQDQ CSCCSPTRTE PMQVALHCTN |
| | | GSVVYHEVLN |
| | 2801 | AMECKCSPRK CSK |
| | Nucleotide Sequence (SEQ ID NO: 1) | |
| Full-length VWF | 1 | ATGATTCCTG CCAGATTTGC CGGGGTGCTG |
| | | CTTGCTCTGG CCCTCATTTT |
| | 51 | GCCAGGGACC CTTTGTGCAG AAGGAACTCG |
| | | CGGCAGGTCA TCCACGGCCC |
| | 101 | GATGCAGCCT TTTCGGAAGT GACTTCGTCA |
| | | ACACCTTTGA TGGGAGCATG |
| | 151 | TACAGCTTTG CGGGATACTG CAGTTACCTC |
| | | CTGGCAGGGG GCTGCCAGAA |
| | 201 | ACGCTCCTTC TCGATTATTG GGGACTTCCA |
| | | GAATGGCAAG AGAGTGAGCC |
| | 251 | TCTCCGTGTA TCTTGGGGAA TTTTTTGACA |
| | | TCCATTTGTT TGTCAATGGT |
| | 301 | ACCGTGACAC AGGGGGACCA AAGAGTCTCC |
| | | ATGCCCTATG CCTCCAAAGG |
| | 351 | GCTGTATCTA GAAACTGAGG CTGGGTACTA |
| | | CAAGCTGTCC GGTGAGGCCT |
| | 401 | ATGGCTTTGT GGCCAGGATC GATGGCAGCG |
| | | GCAACTTTCA AGTCCTGCTG |
| | 451 | TCAGACAGAT ACTTCAACAA GACCTGCGGG |
| | | CTGTGTGGCA ACTTTAACAT |
| | 501 | CTTTGCTGAA GATGACTTTA TGACCCAAGA |
| | | AGGGACCTTG ACCTCGGACC |
| | 551 | CTTATGACTT TGCCAACTCA TGGGCTCTGA |
| | | GCAGTGGAGA ACAGTGGTGT |
| | 601 | GAACGGGCAT CTCCTCCCAG CAGCTCATGC |
| | | AACATCTCCT CTGGGGAAAT |
| | 651 | GCAGAAGGGC CTGTGGGAGC AGTGCCAGCT |
| | | TCTGAAGAGC ACCTCGGTGT |
| | 701 | TTGCCCGCTG CCACCCTCTG GTGGACCCCG |
| | | AGCCTTTTGT GGCCCTGTGT |
| | 751 | GAGAAGACTT TGTGTGAGTG TGCTGGGGGG |
| | | CTGGAGTGCG CCTGCCCTGC |
| | 801 | CCTCCTGGAG TACGCCCGGA CCTGTGCCCA |
| | | GGAGGGAATG GTGCTGTACG |
| | 851 | GCTGGACCGA CCACAGCGCG TGCAGCCCAG |
| | | TGTGCCCTGC TGGTATGGAG |
| | 901 | TATAGGCAGT GTGTGTCCCC TTGCGCCAGG |
| | | ACCTGCCAGA GCCTGCACAT |
| | 951 | CAATGAAATG TGTCAGGAGC GATGCGTGGA |
| | | TGGCTGCAGC TGCCCTGAGG |
| | 1001 | GACAGCTCCT GGATGAAGGC CTCTGCGTGG |
| | | AGAGCACCGA GTGTCCCTGC |
| | 1051 | GTGCATTCCG GAAAGCGCTA CCCTCCCGGC |
| | | ACCTCCCTCT CTCGAGACTG |
| | 1101 | CAACACCTGC ATTTGCCGAA ACAGCCAGTG |
| | | GATCTGCAGC AATGAAGAAT |
| | 1151 | GTCCAGGGGA GTGCCTTGTC ACTGGTCAAT |
| | | CCCACTTCAA GAGCTTTGAC |
| | 1201 | AACAGATACT TCACCTTCAG TGGGATCTGC |
| | | CAGTACCTGC TGGCCCGGGA |
| | 1251 | TTGCCAGGAC CACTCCTTCT CCATTGTCAT |
| | | TGAGACTGTC CAGTGTGCTG |
| | 1301 | ATGACCGCGA CGCTGTGTGC ACCCGCTCCG |
| | | TCACCGTCCG GCTGCCTGGC |
| | 1351 | CTGCACAACA GCCTTGTGAA ACTGAAGCAT |
| | | GGGGCAGGAG TTGCCATGGA |
| | 1401 | TGGCCAGGAC ATCCAGCTCC CCCTCCTGAA |
| | | AGGTGACCTC CGCATCCAGC |
| | 1451 | ATACAGTGAC GGCCTCCGTG CGCCTCAGCT |
| | | ACGGGGAGGA CCTGCAGATG |
| | 1501 | GACTGGGATG GCCGCGGGAG GCTGCTGGTG |
| | | AAGCTGTCCC CCGTCTATGC |
| | 1551 | CGGGAAGACC TGCGGCCTGT GTGGGAATTA |
| | | CAATGGCAAC CAGGGCGACG |
| | 1601 | ACTTCCTTAC CCCCTCTGGG CTGGCRGAGC |
| | | CCCGGGTGGA GGACTTCGGG |
| | 1651 | AACGCCTGGA AGCTGCACGG GGACTGCCAG |
| | | GACCTGCAGA AGCAGCACAG |
| | 1701 | CGATCCCTGC GCCCTCAACC CGCGCATGAC |
| | | CAGGTTCTCC GAGGAGGCGT |
| | 1751 | GCGCGGTCCT GACGTCCCCC ACATTCGAGG |
| | | CCTGCCATCG TGCCGTCAGC |
| | 1801 | CCGCTGCCCT ACCTGCGGAA CTGCCGCTAC |
| | | GACGTGTGCT CCTGCTCGGA |
| | 1851 | CGGCCGCGAG TGCCTGTGCG GCGCCCTGGC |
| | | CAGCTATGCC GCGGCCTGCG |
| | 1901 | CGGGGAGAGG CGTGCGCGTC GCGTGGCGCG |
| | | AGCCAGGCCG CTGTGAGCTG |
| | 1951 | AACTGCCCGA AAGGCCAGGT GTACCTGCAG |
| | | TGCGGGACCC CCTGCAACCT |
| | 2001 | GACCTGCCGC TCTCTCTCTT ACCCGGATGA |
| | | GGAATGCAAT GAGGCCTGCC |
| | 2051 | TGGAGGGCTG CTTCTGCCCC CCAGGGCTCT |
| | | ACATGGATGA GAGGGGGGAC |
| | 2101 | TGCGTGCCCA AGGCCCAGTG CCCCTGTTAC |
| | | TATGACGGTG AGATCTTCCA |
| | 2151 | GCCAGAAGAC ATCTTCTCAG ACCATCACAC |
| | | CATGTGCTAC TGTGAGGATG |
| | 2201 | GCTTCATGCA CTGTACCATG AGTGGAGTCC |
| | | CCGGAAGCTT GCTGCCTGAC |
| | 2251 | GCTGTCCTCA GCAGTCCCCT GTCTCATCGC |
| | | AGCAAAAGGA GCCTATCCTG |
| | 2301 | TCGGCCCCCC ATGGTCAAGC TGGTGTGTCC |
| | | CGCTGACAAC CTGCGGGCTG |
| | 2351 | AAGGGCTCGA GTGTACCAAA ACGTGCCAGA |
| | | ACTATGACCT GGAGTGCATG |
| | 2401 | AGCATGGGCT GTGTCTCTGG CTGCCTCTGC |
| | | CCCCCGGGCA TGGTCCGGCA |
| | 2451 | TGAGAACAGA TGTGTGGCCC TGGAAAGGTG |
| | | TCCCTGCTTC CATCAGGGCA |
| | 2501 | AGGAGTATGC CCCTGGAGAA ACAGTGAAGA |
| | | TTGGCTGCAA CACTTGTGTC |
| | 2551 | TGTCGGGACC GGAAGTGGAA CTGCACAGAC |
| | | CATGTGTGTG ATGCCACGTG |
| | 2601 | CTCCACGATC GGCATGGCCC ACTACCTCAC |
| | | CTTCGACGGG CTCAAATACC |
| | 2651 | TGTTCCCCGG GGAGTGCCAG TACGTTCTGG |
| | | TGCAGGATTA CTGCGGCAGT |
| | 2701 | AACCCTGGGA CCTTTCGGAT CCTAGTGGGG |
| | | AATAAGGGAT GCAGCCACCC |
| | 2751 | CTCAGTGAAA TGCAAGAAAC GGGTCACCAT |
| | | CCTGGTGGAG GGAGGAGAGA |
| | 2801 | TTGAGCTGTT TGACGGGGAG GTGAATGTGA |
| | | AGAGGCCCAT GAAGGATGAG |
| | 2851 | ACTCACTTTG AGGTGGTGGA GTCTGGCCGG |
| | | TACATCATTC TGCTGCTGGG |
| | 2901 | CAAAGCCCTC TCCGTGGTCT GGGACCGCCA |
| | | CCTGAGCATC TCCGTGGTCC |
| | 2951 | TGAAGCAGAC ATACCAGGAG AAAGTGTGTG |
| | | GCCTGTGTGG GAATTTTGAT |
| | 3001 | GGCATCCAGA ACAATGACCT CACCAGCAGC |
| | | AACCTCCAAG TGGAGGAAGA |
| | 3051 | CCCTGTGGAC TTTGGGAACT CCTGGAAAGT |
| | | GAGCTCGCAG TGTGCTGACA |
| | 3101 | CCAGAAAAGT GCCTCTGGAC TCATCCCCTG |
| | | CCACCTGCCA TAACAACATC |
| | 3151 | ATGAAGCAGA CGATGGTGGA TTCCTCCTGT |
| | | AGAATCCTTA CCAGTGACGT |
| | 3201 | CTTCCAGGAC TGCAACAAGC TGGTGGACCC |
| | | CGAGCCATAT CTGGATGTCT |
| | 3251 | GCATTTACGA CACCTGCTCC TGTGAGTCCA |
| | | TTGGGGACTG CGCCTGCTTC |
| | 3301 | TGCGACACCA TTGCTGCCTA TGCCCACGTG |
| | | TGTGCCCAGC ATGGCAAGGT |
| | 3351 | GGTGACCTGG AGGACGGCCA CATTGTGCCC |
| | | CCAGAGCTGC GAGGAGAGGA |
| | 3401 | ATCTCCGGGA GAACGGGTAT GAGTGTGAGT |
| | | GGCGCTATAA CAGCTGTGCA |
| | 3451 | CCTGCCTGTC AAGTCACGTG TCAGCACCCT |
| | | GAGCCACTGG CCTGCCCTGT |
| | 3501 | GCAGTGTGTG GAGGGCTGCC ATGCCCACTG |
| | | CCCTCCAGGG AAAATCCTGG |
| | 3551 | ATGAGCTTTT GCAGACCTGC GTTGACCCTG |
| | | AAGACTGTCC AGTGTGTGAG |
| | 3601 | GTGGCTGGCC GGCGTTTTGC CTCAGGAAAG |
| | | AAAGTCACCT TGAATCCCAG |
| | 3651 | TGACCCTGAG CACTGCCAGA TTTGCCACTG |
| | | TGATGTTGTC AACCTCACCT |
| | 3701 | GTGAAGCCTG CCAGGAGCCG GGAGGCCTGG |
| | | TGGTGCCTCC CACAGATGCC |
| | 3751 | CCGGTGAGCC CCACCACTCT GTATGTGGAG |
| | | GACATCTCGG AACCGCCGTT |
| | 3801 | GCACGATTTC TACTGCAGCA GGCTACTGGA |
| | | CCTGGTCTTC CTGCTGGATG |
| | 3851 | GCTCCTCCAG GCTGTCCGAG GCTGAGTTTG |
| | | AAGTGCTGAA GGCCTTTGTG |
| | 3901 | GTGGACATGA TGGAGCGGCT GCGCATCTCC |
| | | CAGAAGTGGG TCCGCGTGGC |
| | 3951 | CGTGGTGGAG TACCACGACG GCTCCCACGC |
| | | CTACATCGGG CTCAAGGACC |
| | 4001 | GGAAGCGACC GTCAGAGCTG CGGCGCATTG |
| | | CCAGCCAGGT GAAGTATGCG |
| | 4051 | GGCAGCCAGG TGGCCTCCAC CAGCGAGGTC |
| | | TTGAAATACA CACTGTTCCA |
| | 4101 | AATCTTCAGC AAGATCGACC GCCCTGAAGC |
| | | CTCCCGCATC GCCCTGCTCC |
| | 4151 | TGATGGCCAG CCAGGAGCCC CAACGGATGT |
| | | CCCGGAACTT TGTCCGCTAC |
| | 4201 | GTCCAGGGCC TGAAGAAGAA GAAGGTCATT |
| | | GTGATCCCGG TGGGCATTGG |
| | 4251 | GCCCCATGCC AACCTCAAGC AGATCCGCCT |
| | | CATCGAGAAG CAGGCCCCTG |
| | 4301 | AGAACAAGGC CTTCGTGCTG AGCAGTGTGG |
| | | ATGAGCTGGA GCAGCAAAGG |
| | 4351 | GACGAGATCG TTAGCTACCT CTGTGACCTT |
| | | GCCCCTGAAG CCCCTCCTCC |
| | 4401 | TACTCTGCCC CCCGACATGG CACAAGTCAC |
| | | TGTGGGCCCG GGGCTCTTGG |
| | 4451 | GGGTTTCGAC CCTGGGGCCC AAGAGGAACT |
| | | CCATGGTTCT GGATGTGGCG |
| | 4501 | TTCGTCCTGG AAGGATCGGA CAAAATTGGT |
| | | GAAGCCGACT TCAACAGGAG |
| | 4551 | CAAGGAGTTC ATGGAGGAGG TGATTCAGCG |
| | | GATGGATGTG GGCCAGGACA |
| | 4601 | GCATCCACGT CACGGTGCTG CAGTACTCCT |
| | | ACATGGTGAC CGTGGAGTAC |
| | 4651 | CCCTTCAGCG AGGCACAGTC CAAAGGGGAC |
| | | ATCCTGCAGC GGGTGCGAGA |
| | 4701 | GATCCGCTAC CAGGGCGGCA ACAGGACCAA |
| | | CACTGGGCTG GCCCTGCGGT |
| | 4751 | ACCTCTCTGA CCACAGCTTC TTGGTCAGCC |
| | | AGGGTGACCG GGAGCAGGCG |
| | 4801 | CCCAACCTGG TCTACATGGT CACCGGAAAT |
| | | CCTGCCTCTG ATGAGATCAA |
| | 4851 | GAGGCTGCCT GGAGACATCC AGGTGGTGCC |
| | | CATTGGAGTG GGCCCTAATG |
| | 4901 | CCAACGTGCA GGAGCTGGAG AGGATTGGCT |
| | | GGCCCAATGC CCCTATCCTC |
| | 4951 | ATCCAGGACT TTGAGACGCT CCCCCGAGAG |
| | | GCTCCTGACC TGGTGCTGCA |
| | 5001 | GAGGTGCTGC TCCGGAGAGG GGCTGCAGAT |
| | | CCCCACCCTC TCCCCTGCAC |
| | 5051 | CTGACTGCAG CCAGCCCCTG GACGTGATCC |
| | | TTCTCCTGGA TGGCTCCTCC |
| | 5101 | AGTTTCCCAG CTTCTTATTT TGATGAAATG |
| | | AAGAGTTTCG CCAAGGCTTT |
| | 5151 | CATTTCAAAA GCCAATATAG GGCCTCGTCT |
| | | CACTCAGGTG TCAGTGCTGC |
| | 5201 | AGTATGGAAG CATCACCACC ATTGACGTGC |
| | | CATGGAACGT GGTCCCGGAG |
| | 5251 | AAAGCCCATT TGCTGAGCCT TGTGGACGTC |
| | | ATGCAGCGGG AGGGAGGCCC |
| | 5301 | CAGCCAAATC GGGGATGCCT TGGGCTTTGC |
| | | TGTGCGATAC TTGACTTCAG |
| | 5351 | AAATGCATGG TGCCAGGCCG GGAGCCTCAA |
| | | AGGCGGTGGT CATCCTGGTC |
| | 5401 | ACGGACGTCT CTGTGGATTC AGTGGATGCA |
| | | GCAGCTGATG CCGCCAGGTC |
| | 5451 | CAACAGAGTG ACAGTGTTCC CTATTGGAAT |
| | | TGGAGATCGC TACGATGCAG |
| | 5501 | CCCAGCTACG GATCTTGGCA GGCCCAGCAG |
| | | GCGACTCCAA CGTGGTGAAG |
| | 5551 | CTCCAGCGAA TCGAAGACCT CCCTACCATG |
| | | GTCACCTTGG GCAATTCCTT |
| | 5601 | CCTCCACAAA CTGTGCTCTG GATTTGTTAG |
| | | GATTTGCATG GATGAGGATG |
| | 5651 | GGAATGAGAA GAGGCCCGGG GACGTCTGGA |
| | | CCTTGCCAGA CCAGTGCCAC |
| | 5701 | ACCGTGACTT GCCAGCCAGA TGGCCAGACC |
| | | TTGCTGAAGA GTCATCGGGT |
| | 5751 | CAACTGTGAC CGGGGGCTGA GGCCTTCGTG |
| | | CCCTAACAGC CAGTCCCCTG |
| | 5801 | TTAAAGTGGA AGAGACCTGT GGCTGCCGCT |
| | | GGACCTGCCC CTGYGTGTGC |
| | 5851 | ACAGGCAGCT CCACTCGGCA CATCGTGACC |
| | | TTTGATGGGC AGAATTTCAA |
| | 5901 | GCTGACTGGC AGCTGTTCTT ATGTCCTATT |
| | | TCAAAACAAG GAGCAGGACC |
| | 5951 | TGGAGGTGAT TCTCCATAAT GGTGCCTGCA |
| | | GCCCTGGAGC AAGGCAGGGC |
| | 6001 | TGCATGAAAT CCATCGAGGT GAAGCACAGT |
| | | GCCCTCTCCG TCGAGSTGCA |
| | 6051 | CAGTGACATG GAGGTGACGG TGAATGGGAG |
| | | ACTGGTCTCT GTTCCTTACG |
| | 6101 | TGGGTGGGAA CATGGAAGTC AACGTTTATG |
| | | GTGCCATCAT GCATGAGGTC |
| | 6151 | AGATTCAATC ACCTTGGTCA CATCTTCACA |
| | | TTCACTCCAC AAAACAATGA |
| | 6201 | GTTCCAACTG CAGCTCAGCC CCAAGACTTT |
| | | TGCTTCAAAG ACGTATGGTC |
| | 6251 | TGTGTGGGAT CTGTGATGAG AACGGAGCCA |
| | | ATGACTTCAT GCTGAGGGAT |
| | 6301 | GGCACAGTCA CCACAGACTG GAAAACACTT |
| | | GTTCAGGAAT GGACTGTGCA |
| | 6351 | GCGGCCAGGG CAGACGTGCC AGCCCATCCT |
| | | GGAGGAGCAG TGTCTTGTCC |
| | 6401 | CCGACAGCTC CCACTGCCAG GTCCTCCTCT |
| | | TACCACTGTT TGCTGAATGC |
| | 6451 | CACAAGGTCC TGGCTCCAGC CACATTCTAT |
| | | GCCATCTGCC AGCAGGACAG |
| | 6501 | TTGCCACCAG GAGCAAGTGT GTGAGGTGAT |
| | | CGCCTCTTAT GCCCACCTCT |
| | 6551 | GTCGGACCAA CGGGGTCTGC GTTGACTGGA |
| | | GGACACCTGA TTTCTGTGCT |
| | 6601 | ATGTCATGCC CACCATCTCT GGTCTACAAC |
| | | CACTGTGAGC ATGGCTGTCC |
| | 6651 | CCGGCACTGT GATGGCAACG TGAGCTCCTG |
| | | TGGGGACCAT CCCTCCGAAG |
| | 6701 | GCTGTTTCTG CCCTCCAGAT AAAGTCATGT |
| | | TGGAAGGCAG CTGTGTCCCT |
| | 6751 | GAAGAGGCCT GCACTCAGTG CATTGGTGAG |
| | | GATGGAGTCC AGCACCAGTT |
| | 6801 | CCTGGAAGCC TGGGTCCCGG ACCACCAGCC |
| | | CTGTCAGATC TGCACATGCC |
| | 6851 | TCAGCGGGCG GAAGGTCAAC TGCACAACGC |
| | | AGCCCTGCCC CACGGCCAAA |
| | 6901 | GCTCCCACGT GTGGCCTGTG TGAAGTAGCC |
| | | CGCCTCCGCC AGAATGCAGA |
| | 6951 | CCAGTGCTGC CCCGAGTATG AGTGTGTGTG |
| | | TGACCCAGTG AGCTGTGACC |
| | 7001 | TGCCCCCAGT GCCTCACTGT GAACGTGGCC |
| | | TCCAGCCCAC ACTGACCAAC |
| | 7051 | CCTGGCGAGT GCAGACCCAA CTTCACCTGC |
| | | GCCTGCAGGA AGGAGGAGTG |
| | 7101 | CAAAAGAGTG TCCCCACCCT CCTGCCCCCC |
| | | GCACCGTTTG CCCACCCTTC |
| | 7151 | GGAAGACCCA GTGCTGTGAT GAGTATGAGT |
| | | GTGCCTGCAA CTGTGTCAAC |
| | 7201 | TCCACAGTGA GCTGTCCCCT TGGGTACTTG |
| | | GCCTCAACCG CCACCAATGA |
| | 7251 | CTGTGGCTGT ACCACAACCA CCTGCCTTCC |
| | | CGACAAGGTG TGTGTCCACC |
| | 7301 | GAAGCACCAT CTACCCTGTG GGCCAGTTCT |
| | | GGGAGGAGGG CTGCGATGTG |
| | 7351 | TGCACCTGCA CCGACATGGA GGATGCCGTG |
| | | ATGGGCCTCC GCGTGGCCCA |
| | 7401 | GTGCTCCCAG AAGCCCTGTG AGGACAGCTG |
| | | TCGGTCGGGC TTCACTTACG |
| | 7451 | TTCTGCATGA AGGCGAGTGC TGTGGAAGGT |
| | | GCCTGCCATC TGCCTGTGAG |
| | 7501 | GTGGTGACTG GCTCACCGCG GGGGGACTCC |
| | | CAGTCTTCCT GGAAGAGTGT |
| | 7551 | CGGCTCCCAG TGGGCCTCCC CGGAGAACCC |
| | | CTGCCTCATC AATGAGTGTG |
| | 7601 | TCCGAGTGAA GGAGGAGGTC TTTATACAAC |
| | | AAAGGAACGT CTCCTGCCCC |
| | 7651 | CAGCTGGAGG TCCCTGTCTG CCCCTCGGGC |
| | | TTTCAGCTGA GCTGTAAGAC |
| | 7701 | CTCAGCGTGC TGCCCAAGCT GTCGCTGTGA |
| | | GCGCATGGAG GCCTGCATGC |
| | 7751 | TCAATGGCAC TGTCATTGGG CCCGGGAAGA |
| | | CTGTGATGAT CGATGTGTGC |
| | 7801 | ACGACCTGCC GCTGCATGGT GCAGGTGGGG |
| | | GTCATCTCTG GATTCAAGCT |
| | 7851 | GGAGTGCAGG AAGACCACCT GCAACCCCTG |
| | | CCCCCTGGGT TACAAGGAAG |
| | 7901 | AAAATAACAC AGGTGAATGT TGTGGGAGAT |
| | | GTTTGCCTAC GGCTTGCACC |
| | 7951 | ATTCAGCTAA GAGGAGGACA GATCATGACA |
| | | CTGAAGCGTG ATGAGACGCT |
| | 8001 | CCAGGATGGC TGTGATACTC ACTTCTGCAA |
| | | GGTCAATGAG AGAGGAGAGT |
| | 8051 | ACTTCTGGGA GAAGAGGGTC ACAGGCTGCC |
| | | CACCCTTTGA TGAACACAAG |
| | 8101 | TGTCTTGCTG AGGGAGGTAA AATTATGAAA |
| | | ATTCCAGGCA CCTGCTGTGA |
| | 8151 | CACATGTGAG GAGCCTGAGT GCAACGACAT |
| | | CACTGCCAGG CTGCAGTATG |
| | 8201 | TCAAGGTGGG AAGCTGTAAG TCTGAAGTAG |
| | | AGGTGGATAT CCACTACTGC |
| | 8251 | CAGGGCAAAT GTGCCAGCAA AGCCATGTAC |
| | | TCCATTGACA TCAACGATGT |
| | 8301 | GCAGGACCAG TGCTCCTGCT GCTCTCCGAC |
| | | ACGGACGGAG CCCATGCAGG |
| | 8351 | TGGCCCTGCA CTGCACCAAT GGCTCTGTTG |
| | | TGTACCATGA GGTTCTCAAT |
| | 8401 | GCCATGGAGT GCAAATGCTC CCCCAGGAAG |
| | | TGCAGCAAGT GA |

The VWF protein as used herein can comprise a D′ domain and a D3 domain of VWF, wherein the VWF protein binds to FVIII and inhibits binding of endogenous VWF (full-length VWF) to FVIII. The VWF protein comprising the D′ domain and the D3 domain can further comprise a VWF domain selected from an A1 domain, an A2 domain, an A3 domain, a D1 domain, a D2 domain, a D4 domain, a B1 domain, a B2 domain, a B3 domain, a C1 domain, a C2 domain, a CK domain, one or more fragments thereof, or any combination thereof. In one embodiment, a VWF protein comprises, consists essentially of, or consists of: (1) the D′ and D3 domains of VWF or fragments thereof; (2) the D1, D′, and D3 domains of VWF or fragments thereof; (3) the D2, D′, and D3 domains of VWF or fragments thereof; (4) the D1, D2, D′, and D3 domains of VWF or fragments thereof; or (5) the D1, D2, D′, D3, or A1 domains of VWF or fragments thereof. The VWF protein described herein does not contain a VWF clearance receptor binding site. The VWF protein of the present invention can comprise any other sequences linked to or fused to the VWF protein. For example, a VWF protein described herein can further comprise a signal peptide.

In one embodiment, a VWF protein binds to or is associated with a FVIII protein. By binding to or being associated with a FVIII protein, the VWF protein of the invention can protect FVIII from protease cleavage and FVIII activation, stabilizes the heavy chain and light chain of rVIII, and prevents clearance of FVIII by scavenger receptors. In another embodiment, the VWF protein binds to or associates with a FVIII protein and blocks or prevents binding of the FVIII protein to phospholipid and activated Protein C. By preventing or inhibiting binding of the FVIII protein with endogenous, full-length VWF, the VWF protein of the invention reduces the clearance of FVIII by endogenous VWF clearance receptors and thus extends half-life of the FVIII protein. The half-life extension of a FVIII protein is thus due to the association of the FVIII protein with a VWF protein lacking a VWF clearance receptor binding site and thereby shielding and/or protecting of the FVIII protein from endogenous VWF which contains the VWF clearance receptor binding site. The FVIII protein bound to or protected by the VWF protein can also allow recycling of a FVIII protein. By eliminating the VWF clearance pathway receptor binding sites in the full length VWF molecule, the FVIII/VWF heterodimers of the invention are shielded from the VWF clearance pathway, further extending FVIII half-life.

In one embodiment, a VWF protein of the present invention comprises a D′ domain and a D3 domain of VWF, wherein the D′ domain is at least about 60%, 70%, 80%, 85%, 90%, 95%, 96%, 97%, 98%, 99%, or 100% identical to amino acids 764 to 866 of SEQ ID NO: 2, wherein the VWF protein prevents binding of endogenous VWF to FVIII. In another embodiment, a VWF protein comprises a D′ domain and a D3 domain of VWF, wherein the D3 domain is at least 60%, 70%, 80%, 85%, 90%, 95%, 96%, 97%, 98%, 99%, or 100% identical to amino acids 867 to 1240 of SEQ ID NO: 2, wherein the VWF protein prevents binding of endogenous VWF to FVIII. In some embodiments, a VWF protein described herein comprises, consists essentially of, or consists of a D′ domain and a D3 domain of VWF, which are at least 60%, 70%, 80%, 85%, 90%, 95%, 96%, 97%, 98%, 99%, or 100% identical to amino acids 764 to 1240 of SEQ ID NO: 2, wherein the VWF protein prevents binding of endogenous VWF to FVIII. In other embodiments, a VWF protein comprises, consists essentially of, or consists of the D1, D2, D′, and D3 domains at least 60%, 70%, 80%, 85%, 90%, 95%, 96%, 97%, 98%, 99%, or 100% identical to amino acids 23 to 1240 of SEQ ID NO: 2, wherein the VWF protein prevents binding of endogenous VWF to FVIII. In still other embodiments, the VWF protein further comprises a signal peptide operably linked thereto.

In some embodiments, a VWF protein of the invention consists essentially of or consists of (1) the D′D3 domain, the D1D′D3 domain, D2D′D3 domain, or D1D2D′D3 domain and (2) an additional VWF sequence up to about 10 amino acids (e.g., any sequences from amino acids 764 to 1240 of SEQ ID NO: 2 to amino acids 764 to 1250 of SEQ ID NO: 2), up to about 15 amino acids (e.g., any sequences from amino acids 764 to 1240 of SEQ ID NO: 2 to amino acids 764 to 1255 of SEQ ID NO: 2), up to about 20 amino acids (e.g., any sequences from amino acids 764 to 1240 of SEQ ID NO: 2 to amino acids 764 to 1260 of SEQ ID NO: 2), up to about 25 amino acids (e.g., any sequences from amino acids 764 to 1240 of SEQ ID NO: 2 to amino acids 764 to 1265 of SEQ ID NO: 2), or up to about 30 amino acids (e.g., any sequences from amino acids 764 to 1240 of SEQ ID NO: 2 to amino acids 764 to 1260 of SEQ ID NO: 2). In a particular embodiment, the VWF protein comprising or consisting essentially of a D′ domain and a D3 domain is neither amino acids 764 to 1274 of SEQ ID NO: 2 nor the full-length mature VWF. In some embodiments, the D1D2 domain is expressed in trans with the D′D3 domain. In some embodiments, the D1D2 domain is expressed in cis with the D′D3 domain.

In other embodiments, a VWF protein comprising D′D3 domains linked to D1D2 domains further comprises an intracellular processing site, e.g., (a processing site by PACE (furin) or PC5), allowing cleavage of the D1D2 domains from the D′D3 domains upon expression. Non-limiting examples of the intracellular processing sites are disclosed elsewhere herein.

In yet other embodiments, a VWF protein comprises a D′ domain and a D3 domain, but does not comprise an amino acid sequence selected from (1) amino acids 1241 to 2813 of SEQ ID NO: 2, (2) amino acids 1270 to amino acids 2813 of SEQ ID NO: 2, (3) amino acids 1271 to amino acids 2813 of SEQ ID NO: 2, (4) amino acids 1272 to amino acids 2813 of SEQ ID NO: 2, (5) amino acids 1273 to amino acids 2813 of SEQ ID NO: 2, (6) amino acids 1274 to amino acids 2813 of SEQ ID NO: 2, or any combination thereof.

In still other embodiments, a VWF protein of the present invention comprises, consists essentially of, or consists of an amino acid sequence corresponding to a D′ domain, D3 domain, and A1 domain, wherein the amino acid sequence is at least 60%, 70%, 75%, 80%, 85%, 90%, 95%, 96%, 97%, 98%, 99%, or 100% identical to amino acid 764 to 1479 of SEQ ID NO: 2, wherein the VWF protein prevents binding of endogenous VWF to FVIII. In a particular embodiment, the VWF protein is not amino acids 764 to 1274 of SEQ ID NO: 2.

In some embodiments, a VWF protein of the invention comprises a D′ domain and a D3 domain, but does not comprise at least one VWF domain selected from (1) an A1 domain, (2) an A2 domain, (3) an A3 domain, (4) a D4 domain, (5) a B1 domain, (6) a B2 domain, (7) a B3 domain, (8) a C1 domain, (9) a C2 domain, (10) a CK domain, (11) a CK domain and C2 domain, (12) a CK domain, a C2 domain, and a C1 domain, (13) a CK domain, a C2 domain, a C1 domain, a B3 domain, (14) a CK domain, a C2 domain, a C1 domain, a B3 domain, a B2 domain, (15) a CK domain, a C2 domain, a C1 domain, a B3 domain, a B2 domain, and a B1 domain, (16) a CK domain, a C2 domain, a C1 domain, a B3 domain, a B2 domain, a B1 domain, and a D4 domain, (17) a CK domain, a C2 domain, a C1 domain, a B3 domain, a B2 domain, a B1 domain, a D4 domain, and an A3 domain, (18) a CK domain, a C2 domain, a C1 domain, a B3 domain, a B2 domain, a B1 domain, a D4 domain, an A3 domain, and an A2 domain, (19) a CK domain, a C2 domain, a C1 domain, a B3 domain, a B2 domain, a B1 domain, a D4 domain, an A3 domain, an A2 domain, and an A1 domain, or (20) any combination thereof.

In yet other embodiments, a VWF protein comprises D′D3 domains and one or more domains or modules. Examples of such domains or modules include, but are not limited to, the domains and modules disclosed in Zhour et al., Blood published online Apr. 6, 2012: DOI 10.1182/blood-2012-01-405134. For example, the VWF protein can comprise D′D3 domain and one or more domains or modules selected from A1 domain, A2 domain, A3 domain, D4N module, VWD4 module, C8-4 module, TIL-4 module, C1 module, C2 module, C3 module, C4 module, C5 module, C5 module, C6 module, or any combination thereof.

In certain embodiments, a VWF protein of the invention forms a multimer, e.g., dimer, trimer, tetramer, pentamer, hexamer, heptamer, or the higher order multimers. In other embodiments, the VWF protein is a monomer having only one VWF protein. In some embodiments, the VWF protein of the present invention can have one or more amino acid substitutions, deletions, additions, or modifications. In one embodiment, the VWF protein can include amino acid substitutions, deletions, additions, or modifications such that the VWF protein is not capable of forming a disulfide bond or forming a dimer or a multimer. In another embodiment, the amino acid substitution is within the D′ domain and the D3 domain. In a particular embodiment, a VWF protein of the invention contains at least one amino acid substitution at a residue corresponding to residue 1099, residue 1142, or both residues 1099 and 1142 of SEQ ID NO: 2. The at least one amino acid substitution can be any amino acids that are not occurring naturally in the wild type VWF. For example, the amino acid substitution can be any amino acids other than cysteine, e.g., isoleucine, alanine, leucine, asparagine, lysine, aspartic acid, methionine, phenylalanine, glutamic acid, threonine, glutamine, tryptophan, glycine, valine, proline, serine, tyrosine, arginine, or histidine. In another example, the amino acid substitution has one or more amino acids that prevent or inhibit the VWF proteins from forming multimers.

In some embodiments the VWF protein comprises an amino acid substitution from cysteine to alanine at residue 336 corresponding to D′D3 domain of VWF (residue 1099 of SEQ ID NO: 2), and amino acid substitution from cysteine to alanine at residue 379 corresponding to D′D3 domain of VWF (residue 1142 of SEQ ID NO: 2), or both.

In certain embodiments, the VWF protein useful herein can be further modified to improve its interaction with FVIII, e.g., to improve binding affinity to FVIII. As a non-limiting example, the VWF protein comprises a serine residue at the residue corresponding to amino acid 764 of SEQ ID NO: 2 and a lysine residue at the residue corresponding to amino acid 773 of SEQ ID NO: 2. Residues 764 and/or 773 can contribute to the binding affinity of the VWF proteins to FVIII. In other embodiments, the VWF proteins useful for the invention can have other modifications, e.g., the protein can be pegylated, glycosylated, hesylated, or polysialylated.

II.C.3. Heterologous Moiety

A heterologous moiety that can be fused to a VWF protein via a VWF linker or to a FVIII protein via a FVIII linker can be a heterologous polypeptide or a heterologous non-polypeptide moiety. In certain embodiments, the heterologous moiety is a half-life extending molecule which is known in the art and comprises a polypeptide, a non-polypeptide moiety, or the combination of both. A heterologous polypeptide moiety can comprise a FVIII protein, an immunoglobulin constant region or a portion thereof, albumin or a fragment thereof, an albumin binding moiety, transferrin or a fragment thereof, a PAS sequence, a HAP sequence, a derivative or variant thereof, the C-terminal peptide (CTP) of the β subunit of human chorionic gonadotropin, or any combination thereof. In some embodiments, the non-polypeptide binding moiety comprises polyethylene glycol (PEG), polysialic acid, hydroxyethyl starch (HES), a derivative thereof, or any combination thereof. In certain embodiments, there can be one, two, three or more heterologous moieties, which can each be the same or different molecules.

II.C.3.a Immunoglobulin Constant Region or Portion Thereof

An immunoglobulin constant region is comprised of domains denoted CH (constant heavy) domains (CH1, CH2, etc.). Depending on the isotype, (i.e. IgG, IgM, IgA IgD, or IgE), the constant region can be comprised of three or four CH domains. Some isotypes (e.g. IgG) constant regions also contain a hinge region. See Janeway et al. 2001, Immunobiology, Garland Publishing, N.Y., N.Y.

An immunoglobulin constant region or a portion thereof for producing the chimeric protein of the present invention may be obtained from a number of different sources. In some embodiments, an immunoglobulin constant region or a portion thereof is derived from a human immunoglobulin. It is understood, however, that the immunoglobulin constant region or a portion thereof may be derived from an immunoglobulin of another mammalian species, including for example, a rodent (e.g., a mouse, rat, rabbit, guinea pig) or non-human primate (e.g. chimpanzee, macaque) species. Moreover, the immunoglobulin constant region or a portion thereof may be derived from any immunoglobulin class, including IgM, IgG, IgD, IgA and IgE, and any immunoglobulin isotype, including IgG1, IgG2, IgG3 and IgG4. In one embodiment, the human isotype IgG1 is used.

A variety of the immunoglobulin constant region gene sequences (e.g. human constant region gene sequences) are available in the form of publicly accessible deposits. Constant region domains sequence can be selected having a particular effector function (or lacking a particular effector function) or with a particular modification to reduce immunogenicity. Many sequences of antibodies and antibody-encoding genes have been published and suitable Ig constant region sequences (e.g. hinge, CH2, and/or CH3 sequences, or portions thereof) can be derived from these sequences using art recognized techniques. The genetic material obtained using any of the foregoing methods may then be altered or synthesized to obtain polypeptides of the present invention. It will further be appreciated that the scope of this invention encompasses alleles, variants and mutations of constant region DNA sequences.

The sequences of the immunoglobulin constant region or a portion thereof can be cloned, e.g., using the polymerase chain reaction and primers which are selected to amplify the domain of interest. To clone a sequence of the immunoglobulin constant region or a portion thereof from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270).

An immunoglobulin constant region used herein can include all domains and the hinge region or portions thereof. In one embodiment, the immunoglobulin constant region or a portion thereof comprises CH2 domain, CH3 domain, and a hinge region, i.e., an Fc region or an FcRn binding partner.

As used herein, the term “Fc region” is defined as the portion of a polypeptide which corresponds to the Fc region of native immunoglobulin, i.e., as formed by the dimeric association of the respective Fc domains of its two heavy chains. A native Fc region forms a homodimer with another Fc region.

In one embodiment, the “Fc region” refers to the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc domain comprises at least a hinge domain, a CH2 domain, and a CH3 domain.

The Fc region of an immunoglobulin constant region, depending on the immunoglobulin isotype can include the CH2, CH3, and CH4 domains, as well as the hinge region. Chimeric proteins comprising an Fc region of an immunoglobulin bestow several desirable properties on a chimeric protein including increased stability, increased serum half-life (see Capon et al., 1989, Nature 337:525) as well as binding to Fc receptors such as the neonatal Fc receptor (FcRn) (U.S. Pat. Nos. 6,086,875, 6,485,726, 6,030,613; WO 03/077834; US2003-0235536A1), which are incorporated herein by reference in their entireties.

An immunoglobulin constant region or a portion thereof can be an FcRn binding partner. FcRn is active in adult epithelial tissues and expressed in the lumen of the intestines, pulmonary airways, nasal surfaces, vaginal surfaces, colon and rectal surfaces (U.S. Pat. No. 6,485,726). An FcRn binding partner is a portion of an immunoglobulin that binds to FcRn.

The FcRn receptor has been isolated from several mammalian species including humans. The sequences of the human FcRn, monkey FcRn, rat FcRn, and mouse FcRn are known (Story et al. 1994, J. Exp. Med. 180:2377). The FcRn receptor binds IgG (but not other immunoglobulin classes such as IgA, IgM, IgD, and IgE) at relatively low pH, actively transports the IgG transcellularly in a luminal to serosal direction, and then releases the IgG at relatively higher pH found in the interstitial fluids. It is expressed in adult epithelial tissue (U.S. Pat. Nos. 6,485,726, 6,030,613, 6,086,875; WO 03/077834; US2003-0235536A1) including lung and intestinal epithelium (Israel et al. 1997, Immunology 92:69) renal proximal tubular epithelium (Kobayashi et al. 2002, Am. J. Physiol. Renal Physiol. 282:F358) as well as nasal epithelium, vaginal surfaces, and biliary tree surfaces.

FcRn binding partners useful in the present invention encompass molecules that can be specifically bound by the FcRn receptor including whole IgG, the Fc fragment of IgG, and other fragments that include the complete binding region of the FcRn receptor. The region of the Fc portion of IgG that binds to the FcRn receptor has been described based on X-ray crystallography (Burmeister et al. 1994, Nature 372:379). The major contact area of the Fc with the FcRn is near the junction of the CH2 and CH3 domains. Fc-FcRn contacts are all within a single Ig heavy chain. The FcRn binding partners include whole IgG, the Fc fragment of IgG, and other fragments of IgG that include the complete binding region of FcRn. The major contact sites include amino acid residues 248, 250-257, 272, 285, 288, 290-291, 308-311, and 314 of the CH2 domain and amino acid residues 385-387, 428, and 433-436 of the CH3 domain. References made to amino acid numbering of immunoglobulins or immunoglobulin fragments, or regions, are all based on Kabat et al. 1991, Sequences of Proteins of Immunological Interest, U.S. Department of Public Health, Bethesda, Md.

Fc regions or FcRn binding partners bound to FcRn can be effectively shuttled across epithelial barriers by FcRn, thus providing a non-invasive means to systemically administer a desired therapeutic molecule. Additionally, fusion proteins comprising an Fc region or an FcRn binding partner are endocytosed by cells expressing the FcRn. But instead of being marked for degradation, these fusion proteins are recycled out into circulation again, thus increasing the in vivo half-life of these proteins. In certain embodiments, the portions of immunoglobulin constant regions are an Fc region or an FcRn binding partner that typically associates, via disulfide bonds and other non-specific interactions, with another Fc region or another FcRn binding partner to form dimers and higher order multimers.

An FcRn binding partner region is a molecule or a portion thereof that can be specifically bound by the FcRn receptor with consequent active transport by the FcRn receptor of the Fc region. Specifically bound refers to two molecules forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant KA is higher than 10⁶ M⁻¹, or higher than 10⁸ M⁻¹. If necessary, non-specific binding can be reduced without substantially affecting specific binding by varying the binding conditions. The appropriate binding conditions such as concentration of the molecules, ionic strength of the solution, temperature, time allowed for binding, concentration of a blocking agent (e.g. serum albumin, milk casein), etc., may be optimized by a skilled artisan using routine techniques.

Myriad mutants, fragments, variants, and derivatives are described, e.g., in PCT Publication Nos. WO 2011/069164 A2, WO 2012/006623 A2, WO 2012/006635 A2, or WO 2012/006633 A2, all of which are incorporated herein by reference in their entireties.

II.C.3.b. Albumin or Fragment, or Variant Thereof

In certain embodiments, a heterologous moiety linked to the VWF protein via a VWF linker or linked to a FVIII protein via a FVIII linker is albumin or a functional fragment thereof. In some embodiments, the albumin fused to the VWF protein is covalently associated with an albumin fused to a FVIII protein.

Human serum albumin (HSA, or HA), a protein of 609 amino acids in its full-length form, is responsible for a significant proportion of the osmotic pressure of serum and also functions as a carrier of endogenous and exogenous ligands. The term “albumin” as used herein includes full-length albumin or a functional fragment, variant, derivative, or analog thereof. Examples of albumin or the fragments or variants thereof are disclosed in US Pat. Publ. Nos. 2008/0194481A1, 2008/0004206 A1, 2008/0161243 A1, 2008/0261877 A1, or 2008/0153751 A1 or PCT Appl. Publ. Nos. 2008/033413 A2, 2009/058322 A1, or 2007/021494 A2, which are incorporated herein by references in their entireties.

II.C.3.c. Albumin Binding Moiety

In certain embodiments, a heterologous moiety linked to a VWF protein via a VWF linker or to a FVIII protein via a FVIII linker is an albumin binding moiety, which comprises an albumin binding peptide, a bacterial albumin binding domain, an albumin-binding antibody fragment, or any combination thereof. For example, the albumin binding protein can be a bacterial albumin binding protein, an antibody or an antibody fragment including domain antibodies (see U.S. Pat. No. 6,696,245). An albumin binding protein, for example, can be a bacterial albumin binding domain, such as the one of streptococcal protein G (Konig, T. and Skerra, A. (1998) J. Immunol. Methods 218, 73-83). Other examples of albumin binding peptides that can be used as conjugation partner are, for instance, those having a Cys-Xaa₁-Xaa₂-Xaa₃-Xaa₄-Cys consensus sequence, wherein Xaa₁ is Asp, Asn, Ser, Thr, or Trp; Xaa₂ is Asn, Gln, H is, Ile, Leu, or Lys; Xaa₃ is Ala, Asp, Phe, Trp, or Tyr; and Xaa₄ is Asp, Gly, Leu, Phe, Ser, or Thr as described in US patent application 2003/0069395 or Dennis et al. (Dennis et al. (2002) J. Biol. Chem. 277, 35035-35043).

II.C.3.d. PAS Sequence

In other embodiments, a heterologous moiety linked to a VWF protein via a VWF linker or to a FVIII protein via a FVIII linker is a PAS sequence. In one embodiment, a chimeric molecule comprises a VWF protein described herein fused to a PAS sequence via a VWF linker. In another embodiment, a chimeric molecule of the invention comprises a first chain comprising a VWF protein fused to a PAS sequence via a VWF linker and a second chain comprising a FVIII protein and an additional optional PAS sequence, wherein the PAS sequence shields or protects the VWF binding site on the FVIII protein, thereby inhibiting or preventing interaction of the FVIII protein with endogenous VWF. The two PAS sequences can be covalently associated with each other.

A PAS sequence, as used herein, means an amino acid sequence comprising mainly alanine and serine residues or comprising mainly alanine, serine, and proline residues, the amino acid sequence forming random coil conformation under physiological conditions. Accordingly, the PAS sequence is a building block, an amino acid polymer, or a sequence cassette comprising, consisting essentially of, or consisting of alanine, serine, and proline which can be used as a part of the heterologous moiety in the chimeric protein. Yet, the skilled person is aware that an amino acid polymer also may form random coil conformation when residues other than alanine, serine, and proline are added as a minor constituent in the PAS sequence. The term “minor constituent” as used herein means that amino acids other than alanine, serine, and proline may be added in the PAS sequence to a certain degree, e.g., up to about 12%, i.e., about 12 of 100 amino acids of the PAS sequence, up to about 10%, i.e. about 10 of 100 amino acids of the PAS sequence, up to about 9%, i.e., about 9 of 100 amino acids, up to about 8%, i.e., about 8 of 100 amino acids, about 6%, i.e., about 6 of 100 amino acids, about 5%, i.e., about 5 of 100 amino acids, about 4%, i.e., about 4 of 100 amino acids, about 3%, i.e., about 3 of 100 amino acids, about 2%, i.e., about 2 of 100 amino acids, about 1%, i.e., about 1 of 100 of the amino acids. The amino acids different from alanine, serine and proline may be selected from the group consisting of Arg, Asn, Asp, Cys, Gln, Glu, Gly, His, Ile, Leu, Lys, Met, Phe, Thr, Trp, Tyr, and Val.

Under physiological conditions, the PAS sequence stretch forms a random coil conformation and thereby can mediate an increased in vivo and/or in vitro stability to the VWF factor or the protein of coagulation activity. Since the random coil domain does not adopt a stable structure or function by itself, the biological activity mediated by the VWF protein or the FVIII protein to which it is fused is essentially preserved. In other embodiments, the PAS sequences that form random coil domain are biologically inert, especially with respect to proteolysis in blood plasma, immunogenicity, isoelectric point/electrostatic behavior, binding to cell surface receptors or internalization, but are still biodegradable, which provides clear advantages over synthetic polymers such as PEG.

Non-limiting examples of the PAS sequences forming random coil conformation comprise an amino acid sequence selected from the group consisting of ASPAAPAPASPAAPAPSAPA (SEQ ID NO: 32), AAPASPAPAAPSAPAPAAPS (SEQ ID NO: 33), APSSPSPSAPSSPSPASPSS (SEQ ID NO: 34), APSSPSPSAPSSPSPASPS (SEQ ID NO: 35), SSPSAPSPSSPASPSPSSPA (SEQ ID NO: 36), AASPAAPSAPPAAASPAAPSAPPA (SEQ ID NO: 37) and ASAAAPAAASAAASAPSAAA (SEQ ID NO: 38) or any combination thereof. Additional examples of PAS sequences are known from, e.g., US Pat. Publ. No. 2010/0292130 A1 and PCT Appl. Publ. No. WO 2008/155134 A1.

ILC.3.e. HAP Sequence

In certain embodiments, a heterologous moiety linked to a VWF protein via a VWF linker or to a FVIII protein via a FVIII linker is a glycine-rich homo-amino-acid polymer (HAP). The HAP sequence can comprise a repetitive sequence of glycine, which has at least 50 amino acids, at least 100 amino acids, 120 amino acids, 140 amino acids, 160 amino acids, 180 amino acids, 200 amino acids, 250 amino acids, 300 amino acids, 350 amino acids, 400 amino acids, 450 amino acids, or 500 amino acids in length. In one embodiment, the HAP sequence is capable of extending half-life of a moiety fused to or linked to the HAP sequence. Non-limiting examples of the HAP sequence includes, but are not limited to (Gly)ₙ, (Gly₄Ser)ₙ or S(Gly₄Ser)ₙ, wherein n is 1, 2, 3, 4, 5, 6, 7, 8, 9, 10, 11, 12, 13, 14, 15, 16, 17, 18, 19, or 20. In one embodiment, n is 20, 21, 22, 23, 24, 25, 26, 26, 28, 29, 30, 31, 32, 33, 34, 35, 36, 37, 38, 39, or 40. In another embodiment, n is 50, 60, 70, 80, 90, 100, 110, 120, 130, 140, 150, 160, 170, 180, 190, or 200. See, e.g., Schlapschy M et al., Protein Eng. Design Selection, 20: 273-284 (2007).

II.C.3.f. Transferrin or Fragment Thereof

In certain embodiments, a heterologous moiety linked to a VWF protein via a VWF linker or to a FVIII protein via a FVIII linker is transferrin or a fragment thereof. Any transferrin may be used to make chimeric molecules of the invention. As an example, wild-type human Tf (Tf) is a 679 amino acid protein, of approximately 75 KDa (not accounting for glycosylation), with two main domains, N (about 330 amino acids) and C (about 340 amino acids), which appear to originate from a gene duplication. See GenBank accession numbers NM001063, XM002793, M12530, XM039845, XM 039847 and S95936 (www.ncbi.nlm.nih.gov/), all of which are herein incorporated by reference in their entirety. Transferrin comprises two domains, N domain and C domain. N domain comprises two subdomains, N1 domain and N2 domain, and C domain comprises two subdomains, C1 domain and C2 domain.

In one embodiment, the transferrin portion of the chimeric molecule includes a transferrin splice variant. In one example, a transferrin splice variant can be a splice variant of human transferrin, e.g., Genbank Accession AAA61140. In another embodiment, the transferrin portion of the chimeric molecule includes one or more domains of the transferrin sequence, e.g., N domain, C domain, N1 domain, N2 domain, C1 domain, C2 domain or any combination thereof.

II.C.3.g. Polymer, e.g., Polyethylene Glycol (PEG)

In other embodiments, a heterologous moiety attached to a VWF protein via a VWF linker or to a FVIII protein via a FVIII linker is a soluble polymer known in the art, including, but not limited to, polyethylene glycol, ethylene glycol/propylene glycol copolymers, carboxymethylcellulose, dextran, or polyvinyl alcohol. The heterologous moiety such as soluble polymer can be attached to any positions within the chimeric molecule.

In certain embodiments, a chimeric molecule comprises a VWF protein fused to a heterologous moiety (e.g., an Fc region) via a VWF linker, wherein the VWF protein is further linked to PEG. In another embodiment, a chimeric molecule comprises a VWF protein fused to an Fc region via a VWF linker and a FVIII protein, which are associated with each other, wherein the FVIII protein is linked to PEG.

Also provided by the invention are chemically modified derivatives of the chimeric molecule of the invention which may provide additional advantages such as increased solubility, stability and circulating time of the polypeptide, or decreased immunogenicity (see U.S. Pat. No. 4,179,337). The chemical moieties for modification can be selected from water soluble polymers including, but not limited to, polyethylene glycol, ethylene glycol/propylene glycol copolymers, carboxymethylcellulose, dextran, or polyvinyl alcohol. A chimeric molecule may be modified at random positions within the molecule or at the N- or C-terminus, or at predetermined positions within the molecule and may include one, two, three or more attached chemical moieties.

The polymer can be of any molecular weight, and can be branched or unbranched. For polyethylene glycol, in one embodiment, the molecular weight is between about 1 kDa and about 100 kDa for ease in handling and manufacturing. Other sizes may be used, depending on the desired profile (e.g., the duration of sustained release desired, the effects, if any on biological activity, the ease in handling, the degree or lack of antigenicity and other known effects of the polyethylene glycol to a protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 kDa.

In some embodiments, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), each of which is incorporated herein by reference in its entirety.

The number of polyethylene glycol moieties attached to each chimeric molecule (i.e., the degree of substitution) may also vary. For example, the pegylated proteins of the invention may be linked, on average, to 1, 2, 3, 4, 5, 6, 7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

In other embodiments, a FVIII protein used in the invention is conjugated to one or more polymers. The polymer can be water-soluble and covalently or non-covalently attached to Factor VIII or other moieties conjugated to Factor VIII. Non-limiting examples of the polymer can be poly(alkylene oxide), poly(vinyl pyrrolidone), poly(vinyl alcohol), polyoxazoline, or poly(acryloylmorpholine). Additional types of polymer-conjugated FVIII are disclosed in U.S. Pat. No. 7,199,223.

II.C.3.h. Hydroxyethyl Starch (HES)

In certain embodiments, the heterologous moiety linked to a VWF protein via a VWF linker or a FVIII protein via a FVIII linker is a polymer, e.g., hydroxyethyl starch (HES) or a derivative thereof.

Hydroxyethyl starch (HES) is a derivative of naturally occurring amylopectin and is degraded by alpha-amylase in the body. HES is a substituted derivative of the carbohydrate polymer amylopectin, which is present in corn starch at a concentration of up to 95% by weight. HES exhibits advantageous biological properties and is used as a blood volume replacement agent and in hemodilution therapy in the clinics (Sommermeyer et al., Krankenhauspharmazie, 8(8), 271-278 (1987); and Weidler et al., Arzneim-Forschung/Drug Res., 41, 494-498 (1991)).

Amylopectin contains glucose moieties, wherein in the main chain alpha-1,4-glycosidic bonds are present and at the branching sites alpha-1,6-glycosidic bonds are found. The physical-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

HES is mainly characterized by the molecular weight distribution and the degree of substitution. The degree of substitution, denoted as DS, relates to the molar substitution, is known to the skilled people. See Sommermeyer et al., Krankenhauspharmazie, 8(8), 271-278 (1987), as cited above, in particular p. 273.

In one embodiment, hydroxyethyl starch has a mean molecular weight (weight mean) of from 1 to 300 kD, from 2 to 200 kD, from 3 to 100 kD, or from 4 to 70 kD. hydroxyethyl starch can further exhibit a molar degree of substitution of from 0.1 to 3, preferably 0.1 to 2, more preferred, 0.1 to 0.9, preferably 0.1 to 0.8, and a ratio between C2:C6 substitution in the range of from 2 to 20 with respect to the hydroxyethyl groups. A non-limiting example of HES having a mean molecular weight of about 130 kD is a HES with a degree of substitution of 0.2 to 0.8 such as 0.2, 0.3, 0.4, 0.5, 0.6, 0.7, or 0.8, preferably of 0.4 to 0.7 such as 0.4, 0.5, 0.6, or 0.7. In a specific embodiment, HES with a mean molecular weight of about 130 kD is VOLUVEN® from Fresenius. VOLUVEN® is an artificial colloid, employed, e.g., for volume replacement used in the therapeutic indication for therapy and prophylaxis of hypovolaemia. The characteristics of VOLUVEN® are a mean molecular weight of 130,000+/−20,000 D, a molar substitution of 0.4 and a C2:C6 ratio of about 9:1. In other embodiments, ranges of the mean molecular weight of hydroxyethyl starch are, e.g., 4 to 70 kD or 10 to 70 kD or 12 to 70 kD or 18 to 70 kD or 50 to 70 kD or 4 to 50 kD or 10 to 50 kD or 12 to 50 kD or 18 to 50 kD or 4 to 18 kD or 10 to 18 kD or 12 to 18 kD or 4 to 12 kD or 10 to 12 kD or 4 to 10 kD. In still other embodiments, the mean molecular weight of hydroxyethyl starch employed is in the range of from more than 4 kD and below 70 kD, such as about 10 kD, or in the range of from 9 to 10 kD or from 10 to 11 kD or from 9 to 11 kD, or about 12 kD, or in the range of from 11 to 12 kD) or from 12 to 13 kD or from 11 to 13 kD, or about 18 kD, or in the range of from 17 to 18 kD or from 18 to 19 kD or from 17 to 19 kD, or about 30 kD, or in the range of from 29 to 30, or from 30 to 31 kD, or about 50 kD, or in the range of from 49 to 50 kD or from 50 to 51 kD or from 49 to 51 kD.

In certain embodiments, the heterologous moiety can be mixtures of hydroxyethyl starches having different mean molecular weights and/or different degrees of substitution and/or different ratios of C2:C6 substitution. Therefore, mixtures of hydroxyethyl starches may be employed having different mean molecular weights and different degrees of substitution and different ratios of C2:C6 substitution, or having different mean molecular weights and different degrees of substitution and the same or about the same ratio of C2:C6 substitution, or having different mean molecular weights and the same or about the same degree of substitution and different ratios of C2:C6 substitution, or having the same or about the same mean molecular weight and different degrees of substitution and different ratios of C2:C6 substitution, or having different mean molecular weights and the same or about the same degree of substitution and the same or about the same ratio of C2:C6 substitution, or having the same or about the same mean molecular weights and different degrees of substitution and the same or about the same ratio of C2:C6 substitution, or having the same or about the same mean molecular weight and the same or about the same degree of substitution and different ratios of C2:C6 substitution, or having about the same mean molecular weight and about the same degree of substitution and about the same ratio of C2:C6 substitution.

II.C.3.i. Polysialic Acids (PSA)

In certain embodiments, the non-polypeptide heterologous moiety linked to a VWF protein via a VWF linker or to a FVIII protein via a FVIII linker is a polymer, e.g., polysialic acids (PSAs) or a derivative thereof. Polysialic acids (PSAs) are naturally occurring unbranched polymers of sialic acid produced by certain bacterial strains and in mammals in certain cells. Roth J., et al. (1993) in Polysialic Acid: From Microbes to Man, eds. Roth J., Rutishauser U., Troy F. A. (Birkhaüser Verlag, Basel, Switzerland), pp 335-348. They can be produced in various degrees of polymerization from n=about 80 or more sialic acid residues down to n=2 by limited acid hydrolysis or by digestion with neuraminidases, or by fractionation of the natural, bacterially derived forms of the polymer. The composition of different polysialic acids also varies such that there are homopolymeric forms i.e. the alpha-2,8-linked polysialic acid comprising the capsular polysaccharide of E. coli strain K1 and the group-B meningococci, which is also found on the embryonic form of the neuronal cell adhesion molecule (N-CAM). Heteropolymeric forms also exist-such as the alternating alpha-2,8 alpha-2,9 polysialic acid of E. coli strain K92 and group C polysaccharides of N. meningitidis. Sialic acid may also be found in alternating copolymers with monomers other than sialic acid such as group W135 or group Y of N. meningitidis. Polysialic acids have important biological functions including the evasion of the immune and complement systems by pathogenic bacteria and the regulation of glial adhesiveness of immature neurons during foetal development (wherein the polymer has an anti-adhesive function) Cho and Troy, P.N.A.S., USA, 91 (1994) 11427-11431, although there are no known receptors for polysialic acids in mammals. The alpha-2,8-linked polysialic acid of E. coli strain K1 is also known as ‘colominic acid’ and is used (in various lengths) to exemplify the present invention. Various methods of attaching or conjugating polysialic acids to a polypeptide have been described (for example, see U.S. Pat. No. 5,846,951; WO-A-0187922, and US 2007/0191597 A1, which are incorporated herein by reference in their entireties.

II.C.4. XTEN Sequence.

As used here “XTEN sequence” refers to extended length polypeptides with non-naturally occurring, substantially non-repetitive sequences that are composed mainly of small hydrophilic amino acids, with the sequence having a low degree or no secondary or tertiary structure under physiologic conditions. As a chimeric protein partner, XTENs can serve as a carrier, conferring certain desirable pharmacokinetic, physicochemical and pharmaceutical properties when linked to a VWF protein or a FVIII protein of the invention to create a chimeric protein. Such desirable properties include but are not limited to enhanced pharmacokinetic parameters and solubility characteristics. As used herein, “XTEN” specifically excludes antibodies or antibody fragments such as single-chain antibodies or Fc fragments of a light chain or a heavy chain.

In some embodiments, the XTEN sequence of the invention is a peptide or a polypeptide having greater than about 20, 30, 40, 50, 60, 70, 80, 90, 100, 150, 200, 250, 300, 350, 400, 450, 500, 550, 600, 650, 700, 750, 800, 850, 900, 950, 1000, 1200, 1400, 1600, 1800, or 2000 amino acid residues. In certain embodiments, XTEN is a peptide or a polypeptide having greater than about 20 to about 3000 amino acid residues, greater than 30 to about 2500 residues, greater than 40 to about 2000 residues, greater than 50 to about 1500 residues, greater than 60 to about 1000 residues, greater than 70 to about 900 residues, greater than 80 to about 800 residues, greater than 90 to about 700 residues, greater than 100 to about 600 residues, greater than 110 to about 500 residues, or greater than 120 to about 400 residues.

The XTEN sequence of the invention can comprise one or more sequence motif of 9 to 14 amino acid residues or an amino acid sequence at least 80%, 90%, 91%, 92%, 93%, 94%, 95%, 96%, 97%, 98%, or 99% identical to the sequence motif, wherein the motif comprises, consists essentially of, or consists of 4 to 6 types of amino acids selected from the group consisting of glycine (G), alanine (A), serine (S), threonine (T), glutamate (E) and proline (P). See US 2010-0239554 A1.

In some embodiments, the XTEN comprises non-overlapping sequence motifs in which about 80%, or at least about 85%, or at least about 90%, or about 91%, or about 92%, or about 93%, or about 94%, or about 95%, or about 96%, or about 97%, or about 98%, or about 99% or about 100% of the sequence consists of multiple units of non-overlapping sequences selected from a single motif family selected from Table 2A, resulting in a family sequence. As used herein, “family” means that the XTEN has motifs selected only from a single motif category from Table 2A; i.e., AD, AE, AF, AG, AM, AQ, BC, or BD XTEN, and that any other amino acids in the XTEN not from a family motif are selected to achieve a needed property, such as to permit incorporation of a restriction site by the encoding nucleotides, incorporation of a cleavage sequence, or to achieve a better linkage to FVIII or VWF. In some embodiments of XTEN families, an XTEN sequence comprises multiple units of non-overlapping sequence motifs of the AD motif family, or of the AE motif family, or of the AF motif family, or of the AG motif family, or of the AM motif family, or of the AQ motif family, or of the BC family, or of the BD family, with the resulting XTEN exhibiting the range of homology described above. In other embodiments, the XTEN comprises multiple units of motif sequences from two or more of the motif families of Table 2A. These sequences can be selected to achieve desired physical/chemical characteristics, including such properties as net charge, hydrophilicity, lack of secondary structure, or lack of repetitiveness that are conferred by the amino acid composition of the motifs, described more fully below. In the embodiments hereinabove described in this paragraph, the motifs incorporated into the XTEN can be selected and assembled using the methods described herein to achieve an XTEN of about 36 to about 3000 amino acid residues.

TABLE 2A
XTEN Sequence Motifs of 12 Amino
Acids and Motif Families

| Motif | |
|---|---|
| Family* | MOTIF SEQUENCE |
| AD | GESPGGSSGSES (SEQ ID NO: 49) |
| AD | GSEGSSGPGESS (SEQ ID NO: 50) |
| AD | GSSESGSSEGGP (SEQ ID NO: 51) |
| AD | GSGGEPSESGSS (SEQ ID NO: 52) |
| AE, AM | GSPAGSPTSTEE (SEQ ID NO: 53) |
| AE, AM, AQ | GSEPATSGSETP (SEQ ID NO: 54) |
| AE, AM, AQ | GTSESATPESGP (SEQ ID NO: 55) |
| AE, AM, AQ | GTSTEPSEGSAP (SEQ ID NO: 56) |
| AF, AM | GSTSESPSGTAP (SEQ ID NO: 57) |
| AF, AM | GTSTPESGSASP (SEQ ID NO: 58) |
| AF, AM | GTSPSGESSTAP (SEQ ID NO: 59) |
| AF, AM | GSTSSTAESPGP (SEQ ID NO: 60) |
| AG, AM | GTPGSGTASSSP (SEQ ID NO: 61) |
| AG, AM | GSSTPSGATGSP (SEQ ID NO: 62) |
| AG, AM | GSSPSASTGTGP (SEQ ID NO: 63) |
| AG, AM | GASPGTSSTGSP (SEQ ID NO: 64) |
| AQ | GEPAGSPTSTSE (SEQ ID NO: 65) |
| AQ | GTGEPSSTPASE (SEQ ID NO: 66) |
| AQ | GSGPSTESAPTE (SEQ ID NO: 67) |
| AQ | GSETPSGPSETA (SEQ ID NO: 68) |
| AQ | GPSETSTSEPGA (SEQ ID NO: 69) |
| AQ | GSPSEPTEGTSA (SEQ ID NO: 70) |
| BC | GSGASEPTSTEP (SEQ ID NO: 71) |
| BC | GSEPATSGTEPS (SEQ ID NO: 72) |
| BC | GTSEPSTSEPGA (SEQ ID NO: 73) |
| BC | GTSTEPSEPGSA (SEQ ID NO: 74) |
| BD | GSTAGSETSTEA (SEQ ID NO: 75) |
| BD | GSETATSGSETA (SEQ ID NO: 76) |
| BD | GTSTSATSESGA (SEQ ID NO: 77) |
| BD | GTSTEASEGSAS (SEQ ID NO: 78) |

*Denotes individual motif sequences that, when used together in various permutations, results in a “family sequence”

XTEN can have varying lengths for insertion into or linkage to FVIII or VWF or any other components of the chimeric molecule. In one embodiment, the length of the XTEN sequence(s) is chosen based on the property or function to be achieved in the fusion protein. Depending on the intended property or function, XTEN can be short or intermediate length sequence or longer sequence that can serve as carriers. In certain embodiments, the XTEN include short segments of about 6 to about 99 amino acid residues, intermediate lengths of about 100 to about 399 amino acid residues, and longer lengths of about 400 to about 1000 and up to about 3000 amino acid residues. Thus, the XTEN inserted into or linked to FVIII or VWF can have lengths of about 6, about 12, about 36, about 40, about 42, about 72, about 96, about 144, about 288, about 400, about 500, about 576, about 600, about 700, about 800, about 864, about 900, about 1000, about 1500, about 2000, about 2500, or up to about 3000 amino acid residues in length. In other embodiments, the XTEN sequences is about 6 to about 50, about 50 to about 100, about 100 to 150, about 150 to 250, about 250 to 400, about 400 to about 500, about 500 to about 900, about 900 to 1500, about 1500 to 2000, or about 2000 to about 3000 amino acid residues in length. The precise length of an XTEN inserted into or linked to FVIII or VWF can vary without adversely affecting the activity of the FVIII or VWF. In one embodiment, one or more of the XTEN used herein has 36 amino acids, 42 amino acids, 72 amino acids, 144 amino acids, 288 amino acids, 576 amino acids, or 864 amino acids in length and can be selected from one or more of the XTEN family sequences; i.e., AD, AE, AF, AG, AM, AQ, BC or BD.

In some embodiments, the XTEN sequence used in the invention is at least 60%, 70%, 80%, 85%, 90%, 91%, 92%, 93%, 94%, 95%, 96%, 97%, 98%, 99%, or 100% identical to a sequence selected from the group consisting of AE42, AG42, AE48, AM48, AE72, AG72, AE108, AG108, AE144, AF144, AG144, AE180, AG180, AE216, AG216, AE252, AG252, AE288, AG288, AE324, AG324, AE360, AG360, AE396, AG396, AE432, AG432, AE468, AG468, AE504, AG504, AF504, AE540, AG540, AF540, AD576, AE576, AF576, AG576, AE612, AG612, AE624, AE648, AG648, AG684, AE720, AG720, AE756, AG756, AE792, AG792, AE828, AG828, AD836, AE864, AF864, AG864, AM875, AE912, AM923, AM1318, BC864, BD864, AE948, AE1044, AE1140, AE1236, AE1332, AE1428, AE1524, AE1620, AE1716, AE1812, AE1908, AE2004A, AG948, AG1044, AG1140, AG1236, AG1332, AG1428, AG1524, AG1620, AG1716, AG1812, AG1908, and AG2004. See US 2010-0239554 A1.

In one embodiment, the XTEN sequence is at least 60%, 70%, 80%, 90%, 95%, 96%, 97%, 98%, 99% or 100% identical to an amino acid sequence selected from the group consisting of AE42, AE864, AE576, AE288, AE144, AG864, AG576, AG288, AG144, and any combinations thereof. In another embodiment, the XTEN sequence is selected from the group consisting of AE42, AE864, AE576, AE288, AE144, AG864, AG576, AG288, AG144, and any combinations thereof. In a specific embodiment, the XTEN sequence is AE288. The amino acid sequences for certain XTEN sequences of the invention are shown in Table 2B.

TABLE 2B
XTEN Sequences

| XTEN | Amino Acid Sequence |
|---|---|
| AE42 | GAPGSPAGSPTSTEEGTSESATPESGPGSEPATSGSETPASS |
| SEQ ID NO: 39 | |
| AE72 | GAP TSESATPESG PGSEPATSGS ETPGTSESAT PESGPGSEPA |
| SEQ ID NO: 40 | TSGSETPGTS ESATPESGPG TSTEPSEGSA PGASS |
| AE144 | GSEPATSGSETPGTSESATPESGPGSEPATSGSETPGSPAGSPTSTEEGTSTEPSEG |
| SEQ ID NO: 41 | SAPGSEPATSGSETPGSEPATSGSETPGSEPATSGSETPGTSTEPSEGSAPGTSESA |
| | PESGPGSEPATSGSETPGTSTEPSEGSAP |
| AG144 | GTPGSGTASSSPGSSTPSGATGSPGSSPSASTGTGPGSSPSASTGTGPGASPGTSST |
| SEQ ID NO: 42 | GSPGASPGTSSTGSPGSSTPSGATGSPGSSPSASTGTGPGASPGTSSTGSPGSSPSA |
| | STGTGPGTPGSGTASSSPGSSTPSGATGSP |
| AE288 | GTSESATPESGPGSEPATSGSETPGTSESATPESGPGSEPATSGSETPGTSESATPESG |
| SEQ ID NO: 43 | PGTSTEPSEGSAPGSPAGSPTSTEEGTSESATPESGPGSEPATSGSETPGTSESATPES |
| | GPGSPAGSPTSTEEGSPAGSPTSTEEGTSTEPSEGSAPGTSESATPESGPGTSESATPE |
| | SGPGTSESATPESGPGSEPATSGSETPGSEPATSGSETPGSPAGSPTSTEEGTSTEPSE |
| | GSAPGTSTEPSEGSAPGSEPATSGSETPGTSESATPESGPGTSTEPSEGSAP |
| AG288 | PGASPGTSSTGSPGASPGTSSTGSPGTPGSGTASSSPGSSTPSGATGSPGTPGSGTASS |
| SEQ ID NO: 44 | SPGSSTPSGATGSPGTPGSGTASSSPGSSTPSGATGSPGSSTPSGATGSPGSSPSASTG |
| | TGPGSSPSASTGTGPGASPGTSSTGSPGTPGSGTASSSPGSSTPSGATGSPGSSPSAST |
| | GTGPGSSPSASTGTGPGASPGTSSTGSPGASPGTSSTGSPGSSTPSGATGSPGSSPSAS |
| | TGTGPGASPGTSSTGSPGSSPSASTGTGPGTPGSGTASSSPGSSTPSGATGS |
| AE576 | GSPAGSPTSTEEGTSESATPESGPGTSTEPSEGSAPGSPAGSPTSTEEGTSTEPSEGSA |
| SEQ ID NO: 45 | PGTSTEPSEGSAPGTSESATPESGPGSEPATSGSETPGSEPATSGSETPGSPAGSPTST |
| | EEGTSESATPESGPGTSTEPSEGSAPGTSTEPSEGSAPGSPAGSPTSTEEGTSTEPSEG |
| | SAPGTSTEPSEGSAPGTSESATPESGPGTSTEPSEGSAPGTSESATPESGPGSEPATSG |
| | SETPGTSTEPSEGSAPGTSTEPSEGSAPGTSESATPESGPGTSESATPESGPGSPAGSP |
| | TSTEEGTSESATPESGPGSEPATSGSETPGTSESATPESGPGTSTEPSEGSAPGTSTEP |
| | SEGSAPGTSTEPSEGSAPGTSTEPSEGSAPGTSTEPSEGSAPGTSTEPSEGSAPGSPAG |
| | SPTSTEEGTSTEPSEGSAPGTSESATPESGPGSEPATSGSETPGTSESATPESGPGSEP |
| | ATSGSETPGTSESATPESGPGTSTEPSEGSAPGTSESATPESGPGSPAGSPTSTEEGSP |
| | AGSPTSTEEGSPAGSPTSTEEGTSESATPESGPGTSTEPSEGSAP |
| AG576 | PGTPGSGTASSSPGSSTPSGATGSPGSSPSASTGTGPGSSPSASTGTGPGSSTPSGATG |
| SEQ ID NO: 46 | SPGSSTPSGATGSPGASPGTSSTGSPGASPGTSSTGSPGASPGTSSTGSPGTPGSGTAS |
| | SSPGASPGTSSTGSPGASPGTSSTGSPGASPGTSSTGSPGSSPSASTGTGPGTPGSGTA |
| | SSSPGASPGTSSTGSPGASPGTSSTGSPGASPGTSSTGSPGSSTPSGATGSPGSSTPSG |
| | ATGSPGASPGTSSTGSPGTPGSGTASSSPGSSTPSGATGSPGSSTPSGATGSPGSSTPS |
| | GATGSPGSSPSASTGTGPGASPGTSSTGSPGASPGTSSTGSPGTPGSGTASSSPGASPG |
| | TSSTGSPGASPGTSSTGSPGASPGTSSTGSPGASPGTSSTGSPGTPGSGTASSSPGSST |
| | PSGATGSPGTPGSGTASSSPGSSTPSGATGSPGTPGSGTASSSPGSSTPSGATGSPGSS |
| | TPSGATGSPGSSPSASTGTGPGSSPSASTGTGPGASPGTSSTGSPGTPGSGTASSSPGS |
| | STPSGATGSPGSSPSASTGTGPGSSPSASTGTGPGASPGTSSTGS |
| AE864 | GSPAGSPTSTEEGTSESATPESGPGTSTEPSEGSAPGSPAGSPTSTEEGTSTEPSEGSA |
| SEQ ID NO: 47 | PGTSTEPSEGSAPGTSESATPESGPGSEPATSGSETPGSEPATSGSETPGSPAGSPTST |
| | EEGTSESATPESGPGTSTEPSEGSAPGTSTEPSEGSAPGSPAGSPTSTEEGTSTEPSEG |
| | SAPGTSTEPSEGSAPGTSESATPESGPGTSTEPSEGSAPGTSESATPESGPGSEPATSG |
| | SETPGTSTEPSEGSAPGTSTEPSEGSAPGTSESATPESGPGTSESATPESGPGSPAGSP |
| | TSTEEGTSESATPESGPGSEPATSGSETPGTSESATPESGPGTSTEPSEGSAPGTSTEP |
| | SEGSAPGTSTEPSEGSAPGTSTEPSEGSAPGTSTEPSEGSAPGTSTEPSEGSAPGSPAG |
| | SPTSTEEGTSTEPSEGSAPGTSESATPESGPGSEPATSGSETPGTSESATPESGPGSEP |
| | ATSGSETPGTSESATPESGPGTSTEPSEGSAPGTSESATPESGPGSPAGSPTSTEEGSP |
| | AGSPTSTEEGSPAGSPTSTEEGTSESATPESGPGTSTEPSEGSAPGTSESATPESGPGS |
| | EPATSGSETPGTSESATPESGPGSEPATSGSETPGTSESATPESGPGTSTEPSEGSAPG |
| | SPAGSPTSTEEGTSESATPESGPGSEPATSGSETPGTSESATPESGPGSPAGSPTSTEE |
| | GSPAGSPTSTEEGTSTEPSEGSAPGTSESATPESGPGTSESATPESGPGTSESATPESG |
| | PGSEPATSGSETPGSEPATSGSETPGSPAGSPTSTEEGTSTEPSEGSAPGTSTEPSEGS |
| | APGSEPATSGSETPGTSESATPESGPGTSTEPSEGSAP |
| AG864 | GASPGTSSTGSPGSSPSASTGTGPGSSPSASTGTGPGTPGSGTASSSPGSSTPSGATGS |
| SEQ ID NO: 48 | PGSSPSASTGTGPGASPGTSSTGSPGTPGSGTASSSPGSSTPSGATGSPGTPGSGTASS |
| | SPGASPGTSSTGSPGASPGTSSTGSPGTPGSGTASSSPGSSTPSGATGSPGASPGTSST |
| | GSPGTPGSGTASSSPGSSTPSGATGSPGSSPSASTGTGPGSSPSASTGTGPGSSTPSGA |
| | TGSPGSSTPSGATGSPGASPGTSSTGSPGASPGTSSTGSPGASPGTSSTGSPGTPGSGT |
| | ASSSPGASPGTSSTGSPGASPGTSSTGSPGASPGTSSTGSPGSSPSASTGTGPGTPGSG |
| | TASSSPGASPGTSSTGSPGASPGTSSTGSPGASPGTSSTGSPGSSTPSGATGSPGSSTP |
| | SGATGSPGASPGTSSTGSPGTPGSGTASSSPGSSTPSGATGSPGSSTPSGATGSPGSST |
| | PSGATGSPGSSPSASTGTGPGASPGTSSTGSPGASPGTSSTGSPGTPGSGTASSSPGAS |
| | PGTSSTGSPGASPGTSSTGSPGASPGTSSTGSPGASPGTSSTGSPGTPGSGTASSSPGS |
| | STPSGATGSPGTPGSGTASSSPGSSTPSGATGSPGTPGSGTASSSPGSSTPSGATGSPG |
| | SSTPSGATGSPGSSPSASTGTGPGSSPSASTGTGPGASPGTSSTGSPGTPGSGTASSSP |
| | GSSTPSGATGSPGSSPSASTGTGPGSSPSASTGTGPGASPGTSSTGSPGASPGTSSTGS |
| | PGSSTPSGATGSPGSSPSASTGTGPGASPGTSSTGSPGSSPSASTGTGPGTPGSGTASS |
| | SPGSSTPSGATGSPGSSTPSGATGSPGASPGTSSTGSP |

In those embodiments wherein the XTEN component used has less than 100% of its amino acids consisting of 4, 5, or 6 types of amino acid selected from glycine (G), alanine (A), serine (S), threonine (T), glutamate (E) and proline (P), or less than 100% of the sequence consisting of the sequence motifs from Table 2A or the XTEN sequences of Table 2B, the other amino acid residues of the XTEN are selected from any of the other 14 natural L-amino acids, but are preferentially selected from hydrophilic amino acids such that the XTEN sequence contains at least about 90%, 91%, 92%, 93%, 94%, 95%, 96%, 97%, 98%, or at least about 99% hydrophilic amino acids. The XTEN amino acids that are not glycine (G), alanine (A), serine (S), threonine (T), glutamate (E) and proline (P) are either interspersed throughout the XTEN sequence, are located within or between the sequence motifs, or are concentrated in one or more short stretches of the XTEN sequence, e.g., to create a linker between the XTEN and the other components; e.g., VWF protein. In such cases where the XTEN component comprises amino acids other than glycine (G), alanine (A), serine (S), threonine (T), glutamate (E) and proline (P), it is preferred that less than about 2% or less than about 1% of the amino acids be hydrophobic residues such that the resulting sequences generally lack secondary structure, e.g., not having more than 2% alpha helices or 2% beta-sheets, as determined by the methods disclosed herein. Hydrophobic residues that are less favored in construction of XTEN include tryptophan, phenylalanine, tyrosine, leucine, isoleucine, valine, and methionine. Additionally, one can design the XTEN sequences to contain less than 5% or less than 4% or less than 3% or less than 2% or less than 1% or none of the following amino acids: cysteine (to avoid disulfide formation and oxidation), methionine (to avoid oxidation), asparagine and glutamine (to avoid desamidation). Thus, in some embodiments, the XTEN comprising other amino acids in addition to glycine (G), alanine (A), serine (S), threonine (T), glutamate (E) and proline (P) have a sequence with less than 5% of the residues contributing to alpha-helices and beta-sheets as measured by the Chou-Fasman algorithm and have at least 90%, or at least about 95% or more random coil formation as measured by the GOR algorithm.

In further embodiments, the XTEN sequence used in the invention affects the physical or chemical property, e.g., pharmacokinetics, of the chimeric protein of the present invention. The XTEN sequence used in the present invention can exhibit one or more of the following advantageous properties: conformational flexibility, enhanced aqueous solubility, high degree of protease resistance, low immunogenicity, low binding to mammalian receptors, or increased hydrodynamic (or Stokes) radii. In a specific embodiment, the XTEN sequence linked to a FVIII protein in this invention increases pharmacokinetic properties such as longer terminal half-life or increased area under the curve (AUC), so that the chimeric protein described herein stays in vivo for an increased period of time compared to wild type FVIII. In further embodiments, the XTEN sequence used in this invention increases pharmacokinetic properties such as longer terminal half-life or increased area under the curve (AUC), so that FVIII protein stays in vivo for an increased period of time compared to wild type FVIII.

A variety of methods and assays can be employed to determine the physical/chemical properties of proteins comprising the XTEN sequence. Such methods include, but are not limited to analytical centrifugation, EPR, HPLC-ion exchange, HPLC-size exclusion, HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. Additional methods are disclosed in Amau et al., Prot Expr and Purif 48, 1-13 (2006).

Additional examples of XTEN sequences that can be used according to the present invention and are disclosed in US Patent Publication Nos. 2010/0239554 A1, 2010/0323956 A1, 2011/0046060 A1, 2011/0046061 A1, 2011/0077199 A1, or 2011/0172146 A1, or International Patent Publication Nos. WO 2010091122 A1, WO 2010144502 A2, WO 2010144508 A1, WO 2011028228 A1, WO 2011028229 A1, WO 2011028344 A2, or WO2013123457 A1, or International Application Nos. PCT/US2013/049989.

II.C.5. FVIII Protein

A “FVIII protein” as used herein means a functional FVIII polypeptide in its normal role in coagulation, unless otherwise specified. The term a FVIII protein includes a functional fragment, variant, analog, or derivative thereof that retains the function of full-length wild-type Factor VIII in the coagulation pathway. A “FVIII protein” is used interchangeably with FVIII polypeptide (or protein) or FVIII. Examples of the FVIII functions include, but not limited to, an ability to activate coagulation, an ability to act as a cofactor for factor IX, or an ability to form a tenase complex with factor IX in the presence of Ca²⁺ and phospholipids, which then converts Factor X to the activated form Xa. The FVIII protein can be the human, porcine, canine, rat, or murine FVIII protein. In addition, comparisons between FVIII from humans and other species have identified conserved residues that are likely to be required for function (Cameron et al., Thromb. Haemost. 79:317-22 (1998); U.S. Pat. No. 6,251,632).

A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphosholipid antibodies, D-dimer, genetic tests (e.g. factor V Leiden, prothrombin mutation G20210A), dilute Russell's viper venom time (dRVVT), miscellaneous platelet function tests, thromboelastography (TEG or Sonoclot), thromboelastometry (TEM®, e.g, ROTEM®), or euglobulin lysis time (ELT).

The aPTT test is a performance indicator measuring the efficacy of both the “intrinsic” (also referred to the contact activation pathway) and the common coagulation pathways. This test is commonly used to measure clotting activity of commercially available recombinant clotting factors, e.g., FVIII or FIX. It is used in conjunction with prothrombin time (PT), which measures the extrinsic pathway.

ROTEM analysis provides information on the whole kinetics of haemostasis: clotting time, clot formation, clot stability and lysis. The different parameters in thromboelastometry are dependent on the activity of the plasmatic coagulation system, platelet function, fibrinolysis, or many factors which influence these interactions. This assay can provide a complete view of secondary haemostasis.

The FVIII polypeptide and polynucleotide sequences are known, as are many functional fragments, mutants and modified versions. Examples of human FVIII sequences (full-length) are shown as subsequences in SEQ ID NO: 16 or 18.

TABLE 3
Full-length FVIII (FVIII signal peptide
underlined; FVIII heavy chain is double
underlined; B domain is italicized; and
FVIII light chain is in plain text)

Signal Peptide: (SEQ ID NO: 15)
MQIELSTCFFLCLLRFCFS
Mature Factor VIII (SEQ ID NO: 16)*
ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQRLKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APEERSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
TRQKQFNATIIPENDIEKTDPWFAHRTPMPKIQNVSSSDLLMLLRQSPTP
HGLSLSDLQEAKYETFSDDPSPGAIDSNNSLSEMTHFRPQLHHSGDMVFT
PESGLQLRLNEKLGTTAATELKKLDFKVSSTSNNLISTIPSDNLAAGTDN
TSSLGPPSMPVHYDSQLDTTLFGKKSSPLTESGGPLSLSEENNDSKLLES
GLMNSQESSWGKNVSSTESGRLFKGKRAHGPALLTKDNALFKVSISLLKT
NKTSNNSATNRKTHIDGPSLLIENSPSVWQNILESDTEFKKVTPLIHDRM
LMDKNATALRLNHMSNKTTSSKNMEMVQQKKEGPIPPDAQNPDMSFFKML
FLPESARWIQRTHGKNSLNSGQGPSPKQLVSLGPEKSVEGQNFLSEKNKV
VVGKGEFTKDVGLKEMVFPSSRNLFLTNLDNLHENNTHNQEKKIQEEIEK
KETLIQENVVLPQIHTVTGTKNFMKNLFLLSTRQNVEGSYDGAYAPVLQD
FRSLNDSTNRTKKHTAHFSKKGEEENLEGLGNQTKQIVEKYACTTRISPN
TSQQNFVTQRSKRALKQFRLPLEETELEKRIIVDDTSTQWSKNMKHLTPS
TLTQIDYNEKEKGAITQSPLSDCLTRSHSIPQANRSPLPIAKVSSFPSIR
PIYLTRVLFQDNSSHLPAASYRKKDSGVQESSHFLQGAKKNNLSLAILTL
EMTGDQREVGSLGTSATNSVTYKKVENTVLPKPDLPKTSGKVELLPKVHI
YQKDLFPTETSNGSPGHLDLVEGSLLQGTEGAIKWNEANRPGKVPFLRVA
TESSAKTPSKLLDPLAWDNHYGTQIPKEEWKSQEKSPEKTAFKKKDTILS
LNACESNGAIAAINEGQNKPEIEVTWAKQGRTERLCSQNPPVLKRHQREI
TRTTLQSDQEEIDYDDTISVEMKKEDFDIYDEDENQSPRSFQKKTRHYFI
AAVERLWDYGMSSSPHVLRNRAQSGSVPQFKKVVFQEFTDGSFTQPLYRG
ELNEHLGLLGPYIRAEVEDNIMVTFRNQASRPYSFYSSLISYEEDQRQGA
EPRKNFVKPNETKTYFWKVQHHMAPTKDEFDCKAWAYFSDVDLEKDVHSG
LIGPLLVCHTNTLNPAHGRQVTVQEFALFFTIFDETKSWYFTENMERNCR
APCNIQMEDPTFKENYRFHAINGYIMDTLPGLVMAQDQRIRWYLLSMGSN
ENIHSIHFSGHVFTVRKKEEYKMALYNLYPGVFETVEMLPSKAGIWRVEC
LIGEHLHAGMSTLFLVYSNKCQTPLGMASGHYRDFQITASGQYGQWAPKL
ARLHYSGSINAWSTKEPFSWIKVDLLAPMIIHGIKTQGARQKFSSLYISQ
FIIMYSLDGKKWQTYRGNSTGTLMVFFGNVDSSGIKHNIFNPPIIARYIR
LHPTHYSIRSTLRMELMGCDLNSCSMPLGMESKAISDAQITASSYFTNMF
ATWSPSKARLHLQGRSNAWRPQVNNPKEWLQVDFQKTMKVTGVTTQGVKS
LLTSMYVKEFLISSSQDGHQWTLFFQNGKVKVFQGNQDSFTPVVNSLDPP
LLTRYLRIHPQSWVHQIALRMEVLGCEAQDLY

TABLE 4
Nucleotide Sequence Encoding Full-Length FVIII (SEQ ID NO: 17)*

| | |
|---|---|
| 661 | ATG CAAATAGAGC TCTCCACCTG |
| 721 | CTTCTTTCTG TGCCTTTTGC GATTCTGCTT TAGTGCCACC AGAAGATACT ACCTGGGTGC |
| 781 | AGTGGAACTG TCATGGGACT ATATGCAAAG TGATCTCGGT GAGCTGCCTG TGGACGCAAG |
| 841 | ATTTCCTCCT AGAGTGCCAA AATCTTTTCC ATTCAACACC TCAGTCGTGT ACAAAAAGAC |
| 901 | TCTGTTTGTA GAATTCACGG ATCACCTTTT CAACATCGCT AAGCCAAGGC CACCCTGGAT |
| 961 | GGGTCTGCTA GGTCCTACCA TCCAGGCTGA GGTTTATGAT ACAGTGGTCA TTACACTTAA |
| 1021 | GAACATGGCT TCCCATCCTG TCAGTCTTCA TGCTGTTGGT GTATCCTACT GGAAAGCTTC |
| 1081 | TGAGGGAGCT GAATATGATG ATCAGACCAG TCAAAGGGAG AAAGAAGATG ATAAAGTCTT |
| 1141 | CCCTGGTGGA AGCCATACAT ATGTCTGGCA GGTCCTGAAA GAGAATGGTC CAATGGCCTC |
| 1201 | TGACCCACTG TGCCTTACCT ACTCATATCT TTCTCATGTG GACCTGGTAA AAGACTTGAA |
| 1261 | TTCAGGCCTC ATTGGAGCCC TACTAGTATG TAGAGAAGGG AGTCTGGCCA AGGAAAAGAC |
| 1321 | ACAGACCTTG CACAAATTTA TACTACTTTT TGCTGTATTT GATGAAGGGA AAAGTTGGCA |
| 1381 | CTCAGAAACA AAGAACTCCT TGATGCAGGA TAGGGATGCT GCATCTGCTC GGGCCTGGCC |
| 1441 | TAAAATGCAC ACAGTCAATG GTTATGTAAA CAGGTCTCTG CCAGGTCTGA TTGGATGCCA |
| 1501 | CAGGAAATCA GTCTATTGGC ATGTGATTGG AATGGGCACC ACTCCTGAAG TGCACTCAAT |
| 1561 | ATTCCTCGAA GGTCACACAT TTCTTGTGAG GAACCATCGC CAGGCGTCCT TGGAAATCTC |
| 1621 | GCCAATAACT TTCCTTACTG CTCAAACACT CTTGATGGAC CTTGGACAGT TTCTACTGTT |
| 1681 | TTGTCATATC TCTTCCCACC AACATGATGG CATGGAAGCT TATGTCAAAG TAGACAGCTG |
| 1741 | TCCAGAGGAA CCCCAACTAC GAATGAAAAA TAATGAAGAA GCGGAAGACT ATGATGATGA |
| 1801 | TCTTACTGAT TCTGAAATGG ATGTGGTCAG GTTTGATGAT GACAACTCTC CTTCCTTTAT |
| 1861 | CCAAATTCGC TCAGTTGCCA AGAAGCATCC TAAAACTTGG GTACATTACA TTGCTGCTGA |
| 1921 | AGAGGAGGAC TGGGACTATG CTCCCTTAGT CCTCGCCCCC GATGACAGAA GTTATAAAAG |
| 1981 | TCAATATTTG AACAATGGCC CTCAGCGGAT TGGTAGGAAG TACAAAAAAG TCCGATTTAT |
| 2041 | GGCATACACA GATGAAACCT TTAAGACTCG TGAAGCTATT CAGCATGAAT CAGGAATCTT |
| 2101 | GGGACCTTTA CTTTATGGGG AAGTTGGAGA CACACTGTTG ATTATATTTA AGAATCAAGC |
| 2161 | AAGCAGACCA TATAACATCT ACCCTCACGG AATCACTGAT GTCCGTCCTT TGTATTCAAG |
| 2221 | GAGATTACCA AAAGGTGTAA AACATTTGAA GGATTTTCCA ATTCTGCCAG GAGAAATATT |
| 2281 | CAAATATAAA TGGACAGTGA CTGTAGAAGA TGGGCCAACT AAATCAGATC CTCGGTGCCT |
| 2341 | GACCCGCTAT TACTCTAGTT TCGTTAATAT GGAGAGAGAT CTAGCTTCAG GACTCATTGG |
| 2401 | CCCTCTCCTC ATCTGCTACA AAGAATCTGT AGATCAAAGA GGAAACCAGA TAATGTCAGA |
| 2461 | CAAGAGGAAT GTCATCCTGT TTTCTGTATT TGATGAGAAC CGAAGCTGGT ACCTCACAGA |
| 2521 | GAATATACAA CGCTTTCTCC CCAATCCAGC TGGAGTGCAG CTTGAGGATC CAGAGTTCCA |
| 2581 | AGCCTCCAAC ATCATGCACA GCATCAATGG CTATGTTTTT GATAGTTTGC AGTTGTCAGT |
| 2641 | TTGTTTGCAT GAGGTGGCAT ACTGGTACAT TCTAAGCATT GGAGCACAGA CTGACTTCCT |
| 2701 | TTCTGTCTTC TTCTCTGGAT ATACCTTCAA ACACAAAATG GTCTATGAAG ACACACTCAC |
| 2761 | CCTATTCCCA TTCTCAGGAG AAACTGTCTT CATGTCGATG GAAAACCCAG GTCTATGGAT |
| 2821 | TCTGGGGTGC CACAACTCAG ACTTTCGGAA CAGAGGCATG ACCGCCTTAC TGAAGGTTTC |
| 2881 | TAGTTGTGAC AAGAACACTG GTGATTATTA CGAGGACAGT TATGAAGATA TTTCAGCATA |
| 2941 | CTTGCTGAGT AAAAACAATG CCATTGAACC AAGAAGCTTC TCCCAGAATT CAAGACACCC |
| 3001 | TAGCACTAGG CAAAAGCAAT TTAATGCCAC CACAATTCCA GAAAATGACA TAGAGAAGAC |
| 3061 | TGACCCTTGG TTTGCACACA GAACACCTAT GCCTAAAATA CAAAATGTCT CCTCTAGTGA |
| 3121 | TTTGTTGATG CTCTTGCGAC AGAGTCCTAC TCCACATGGG CTATCCTTAT CTGATCTCCA |
| 3181 | AGAAGCCAAA TATGAGACTT TTTCTGATGA TCCATCACCT GGAGCAATAG ACAGTAATAA |
| 3241 | CAGCCTGTCT GAAATGACAC ACTTCAGGCC ACAGCTCCAT CACAGTGGGG ACATGGTATT |
| 3301 | TACCCCTGAG TCAGGCCTCC AATTAAGATT AAATGAGAAA CTGGGGACAA CTGCAGCAAC |
| 3361 | AGAGTTGAAG AAACTTGATT TCAAAGTTTC TAGTACATCA AATAATCTGA TTTCAACAAT |
| 3421 | TCCATCAGAC AATTTGGCAG CAGGTACTGA TAATACAAGT TCCTTAGGAC CCCCAAGTAT |
| 3481 | GCCAGTTCAT TATGATAGTC AATTAGATAC CACTCTATTT GGCAAAAAGT CATCTCCCCT |
| 3541 | TACTGAGTCT GGTGGACCTC TGAGCTTGAG TGAAGAAAAT AATGATTCAA AGTTGTTAGA |
| 3601 | ATCAGGTTTA ATGAATAGCC AAGAAAGTTC ATGGGGAAAA AATGTATCGT CAACAGAGAG |
| 3661 | TGGTAGGTTA TTTAAAGGGA AAAGAGCTCA TGGACCTGCT TTGTTGACTA AAGATAATGC |
| 3721 | CTTATTCAAA GTTAGCATCT CTTTGTTAAA GACAAACAAA ACTTCCAATA ATTCAGCAAC |
| 3781 | TAATAGAAAG ACTCACATTG ATGGCCCATC ATTATTAATT GAGAATAGTC CATCAGTCTG |
| 3841 | GCAAAATATA TTAGAAAGTG ACACTGAGTT TAAAAAAGTG ACACCTTTGA TTCATGACAG |
| 3901 | AATGCTTATG GACAAAAATG CTACAGCTTT GAGGCTAAAT CATATGTCAA ATAAAACTAC |
| 3961 | TTCATCAAAA AACATGGAAA TGGTCCAACA GAAAAAAGAG GGCCCCATTC CACCAGATGC |
| 4021 | ACAAAATCCA GATATGTCGT TCTTTAAGAT GCTATTCTTG CCAGAATCAG CAAGGTGGAT |
| 4081 | ACAAAGGACT CATGGAAAGA ACTCTCTGAA CTCTGGGCAA GGCCCCAGTC CAAAGCAATT |
| 4141 | AGTATCCTTA GGACCAGAAA AATCTGTGGA AGGTCAGAAT TTCTTGTCTG AGAAAAACAA |
| 4201 | AGTGGTAGTA GGAAAGGGTG AATTTACAAA GGACGTAGGA CTCAAAGAGA TGGTTTTTCC |
| 4261 | AAGCAGCAGA AACCTATTTC TTACTAACTT GGATAATTTA CATGAAAATA ATACACACAA |
| 4321 | TCAAGAAAAA AAAATTCAGG AAGAAATAGA AAAGAAGGAA ACATTAATCC AAGAGAATGT |
| 4381 | AGTTTTGCCT CAGATACATA CAGTGACTGG CACTAAGAAT TTCATGAAGA ACCTTTTCTT |
| 4441 | ACTGAGCACT AGGCAAAATG TAGAAGGTTC ATATGACGGG GCATATGCTC CAGTACTTCA |
| 4501 | AGATTTTAGG TCATTAAATG ATTCAACAAA TAGAACAAAG AAACACACAG CTCATTTCTC |
| 4561 | AAAAAAAGGG GAGGAAGAAA ACTTGGAAGG CTTGGGAAAT CAAACCAAGC AAATTGTAGA |
| 4621 | GAAATATGCA TGCACCACAA GGATATCTCC TAATACAAGC CAGCAGAATT TTGTCACGCA |
| 4681 | ACGTAGTAAG AGAGCTTTGA AACAATTCAG ACTCCCACTA GAAGAAACAG AACTTGAAAA |
| 4741 | AAGGATAATT GTGGATGACA CCTCAACCCA GTGGTCCAAA AACATGAAAC ATTTGACCCC |
| 4801 | GAGCACCCTC ACACAGATAG ACTACAATGA GAAGGAGAAA GGGGCCATTA CTCAGTCTCC |
| 4861 | CTTATCAGAT TGCCTTACGA GGAGTCATAG CATCCCTCAA GCAAATAGAT CTCCATTACC |
| 4921 | CATTGCAAAG GTATCATCAT TTCCATCTAT TAGACCTATA TATCTGACCA GGGTCCTATT |
| 4981 | CCAAGACAAC TCTTCTCATC TTCCAGCAGC ATCTTATAGA AAGAAAGATT CTGGGGTCCA |
| 5041 | AGAAAGCAGT CATTTCTTAC AAGGAGCCAA AAAAAATAAC CTTTCTTTAG CCATTCTAAC |
| 5101 | CTTGGAGATG ACTGGTGATC AAAGAGAGGT TGGCTCCCTG GGGACAAGTG CCACAAATTC |
| 5161 | AGTCACATAC AAGAAAGTTG AGAACACTGT TCTCCCGAAA CCAGACTTGC CCAAAACATC |
| 5221 | TGGCAAAGTT GAATTGCTTC CAAAAGTTCA CATTTATCAG AAGGACCTAT TCCCTACGGA |
| 5281 | AACTAGCAAT GGGTCTCCTG GCCATCTGGA TCTCGTGGAA GGGAGCCTTC TTCAGGGAAC |
| 5341 | AGAGGGAGCG ATTAAGTGGA ATGAAGGAAA CAGACCTGGA AAAGTTCCCT TTCTGAGAGT |
| 5401 | AGCAACAGAA AGCTCTGCAA AGACTCCCTC CAAGCTATTG GATCCTCTTG CTTGGGATAA |
| 5461 | CCACTATGGT ACTCAGATAC CAAAAGAAGA GTGGAAATCC CAAGAGAAGT CACCAGAAAA |
| 5521 | AACAGCTTTT AAGAAAAAGG ATACCATTTT GTCCCTGAAC GCTTGTGAAA GCAATCATGC |
| 5581 | AATAGCAGCA ATAAATGAGG GACAAAATAA GCCCGAAATA GAAGTCACCT GGGCXAAGCA |
| 5641 | AGGTAGGACT GAAAGGCTGT GCTCTCAAAA CCCACCAGTC TTGAAACGCC ATCAACGGGA |
| 5701 | AATAACTCGT ACTACTCTTC AGTCAGATCA AGAGGAAATT GACTATGATG ATACCATATC |
| 5761 | AGTTGAAATG AAGAAGGAAG ATTTTGACAT TTATGATGAG GATGAAAATC AGAGCCCCCG |
| 5821 | CAGCTTTCAA AAGAAAACAC GACACTATTT TATTGCTGCA GTGGAGAGGC TCTGGGATTA |
| 5881 | TGGGATGAGT AGCTCCCCAC ATGTTCTAAG AAACAGGGCT CAGAGTGGCA GTGTCCCTCA |
| 5941 | GTTCAAGAAA GTTGTTTTCC AGGAATTTAC TGATGGCTCC TTTACTCAGC CCTTATACCG |
| 6001 | TGGAGAACTA AATGAACATT TGGGACTCCT GGGGCCATAT ATAAGAGCAG AAGTTGAAGA |
| 6061 | TAATATCATG GTAACTTTCA GAAATCAGGC CTCTCGTCCC TATTCCTTCT ATTCTAGCCT |
| 6121 | TATTTCTTAT GAGGAAGATC AGAGGCAAGG AGCAGAACCT AGAAAAAACT TTGTCAAGCC |
| 6181 | TAATGAAACC AAAACTTACT TTTGGAAAGT GCAACATCAT ATGGCACCCA CTAAAGATGA |
| 6241 | GTTTGACTGC AAAGCCTGGG CTTATTTCTC TGATGTTGAC CTGGAAAAAG ATGTGCACTC |
| 6301 | AGGCCTGATT GGACCCCTTC TGGTCTGCCA CACTAACACA CTGAACCCTG CTCATGGGAG |
| 6361 | ACAAGTGACA GTACAGGAAT TTGCTCTGTT TTTCACCATC TTTGATGAGA CCAAAAGCTG |
| 6421 | GTACTTCACT GAAAATATGG AAAGAAACTG CAGGGCTCCC TGCAATATCC AGATGGAAGA |
| 6481 | TCCCACTTTT AAAGAGAATT ATCGCTTCCA TGCAATCAAT GGCTACATAA TGGATACACT |
| 6541 | ACCTGGCTTA GTAATGGCTC AGGATCAAAG GATTCGATGG TATCTGCTCA GCATGGGCAG |
| 6601 | CAATGAAAAC ATCCATTCTA TTCATTTCAG TGGACATGTG TTCACTGTAC GAAAAAAAGA |
| 6661 | GGAGTATAAA ATGGCACTGT ACAATCTCTA TCCAGGTGTT TTTGAGACAG TGGAAATGTT |
| 6721 | ACCATCCAAA GCTGGAATTT GGCGGGTGGA ATGCCTTATT GGCGAGCATC TACATGCTGG |
| 6781 | GATGAGCACA CTTTTTCTGG TGTACAGCAA TAAGTGTCAG ACTCCCCTGG GAATGGCTTC |
| 6841 | TGGACACATT AGAGATTTTC AGATTACAGC TTCAGGACAA TATGGACAGT GGGCCCCAAA |
| 6901 | GCTGGCCAGA CTTCATTATT CCGGATCAAT CAATGCCTGG AGCACCAAGG AGCGCTTTTC |
| 6961 | TTGGATCAAG GTGGATCTGT TGGCACCAAT GATTATTCAC GGCATCAAGA CCCAGGGTGC |
| 7021 | CCGTCAGAAG TTCTCCAGCC TCTACATCTC TCAGTTTATC ATCATGTATA GTCTTGATGG |
| 7081 | GAAGAAGTGG CAGACTTATC GAGGAAATTC CACTGGAACC TTAATGGTCT TCTTTGGCAA |
| 7141 | TGTGGATTCA TCTGGGATAA AACACAATAT TTTTAACCCT CCAATTATTG CTCGATACAT |
| 7201 | CCGTTTGCAC CCAACTCATT ATAGCATTCG CAGCACTCTT CGCATGGAGT TGATGGGCTG |
| 7261 | TGATTTAAAT AGTTGCAGCA TGCCATTGGG AATGGAGAGT AAAGCAATAT CAGATGCACA |
| 7321 | GATTACTGCT TCATCCTACT TTACCAATAT GTTTGCCACC TGGTCTCCTT CAAAAGCTCG |
| 7381 | ACTTCACCTC CAAGGGAGGA GTAATGCCTG GAGACCTCAG GTGAATAATC CAAAAGAGTG |
| 7441 | GCTGCAAGTG GACTTCCAGA AGACAATGAA AGTCACAGGA GTAACTACTC AGGGAGTAAA |
| 7501 | ATCTCTGCTT ACCAGCATGT ATGTGAAGGA GTTCCTCATC TCCAGCAGTC AAGATGGCCA |
| 7561 | TCAGTGGACT CTCTTTTTTC AGAATGGCAA AGTAAAGGTT TTTCAGGGAA ATCAAGACTC |
| 7621 | CTTCACACCT GTGGTGAACT CTCTAGACCC ACCGTTACTG ACTCGCTACC TTCGAATTCA |
| 7681 | CCCCCAGAGT TGGGTGCACC AGATTGCCCT GAGGATGGAG GTTCTGGGCT GCGAGGCACA |
| 7741 | GGACCTCTAC |

*The underlined nucleic acids encode a signal peptide.

FVIII polypeptides include full-length FVIII, full-length FVIII minus Met at the N-terminus, mature FVIII (minus the signal sequence), mature FVIII with an additional Met at the N-terminus, and/or FVIII with a full or partial deletion of the B domain. In certain embodiments, FVIII variants include B domain deletions, whether partial or full deletions.

The human FVIII gene was isolated and expressed in mammalian cells (Toole, J. J., et al., Nature 312:342-347 (1984); Gitschier, J., et al., Nature 312:326-330 (1984); Wood, W. I., et al., Nature 312:330-337 (1984); Vehar, G. A., et al., Nature 312:337-342 (1984); WO 87/04187; WO 88/08035; WO 88/03558; and U.S. Pat. No. 4,757,006). The FVIII amino acid sequence was deduced from cDNA as shown in U.S. Pat. No. 4,965,199. In addition, partially or fully B-domain deleted FVIII is shown in U.S. Pat. Nos. 4,994,371 and 4,868,112. In some embodiments, the human FVIII B-domain is replaced with the human Factor V B-domain as shown in U.S. Pat. No. 5,004,803. The cDNA sequence encoding human Factor VIII and amino acid sequence are shown in SEQ ID NOs: 1 and 2, respectively, of U.S. Pat. No. 7,211,559.

The porcine FVIII sequence is published in Toole, J. J., et al., Proc. Natl. Acad. Sci. USA 83:5939-5942 (1986). Further, the complete porcine cDNA sequence obtained from PCR amplification of FVIII sequences from a pig spleen cDNA library has been reported in Healey, J. F., et al., Blood 88:4209-4214 (1996). Hybrid human/porcine FVIII having substitutions of all domains, all subunits, and specific amino acid sequences were disclosed in U.S. Pat. No. 5,364,771 by Lollar and Runge, and in WO 93/20093. More recently, the nucleotide and corresponding amino acid sequences of the A1 and A2 domains of porcine FVIII and a chimeric FVIII with porcine A1 and/or A2 domains substituted for the corresponding human domains were reported in WO 94/11503. U.S. Pat. No. 5,859,204, Lollar, J. S., also discloses the porcine cDNA and deduced amino acid sequences. U.S. Pat. No. 6,458,563 discloses a B-domain-deleted porcine FVIII.

U.S. Pat. No. 5,859,204 to Lollar, J. S. reports functional mutants of FVIII having reduced antigenicity and reduced immunoreactivity. U.S. Pat. No. 6,376,463 to Lollar, J. S. also reports mutants of FVIII having reduced immunoreactivity. US Appl. Publ. No. 2005/0100990 to Saenko et al. reports functional mutations in the A2 domain of FVIII.

In one embodiment, the FVIII protein (or FVIII portion of a chimeric protein) may be at least 50%, 60%, 70%, 80%, 90%, 95%, 96%, 97%, 98%, 99%, or 100% identical to a FVIII amino acid sequence of amino acids 1 to 1438 of SEQ ID NO: 18 or amino acids 1 to 2332 of SEQ ID NO: 16 (without a signal sequence), wherein the FVIII has a clotting activity, e.g., activates Factor IX as a cofactor to convert Factor X to activated Factor X. The FVIII (or FVIII portion of a chimeric protein) may be identical to a FVIII amino acid sequence of amino acids 1 to 1438 of SEQ ID NO: 18 or amino acids 1 to 2332 of SEQ ID NO: 16 (without a signal sequence). The FVIII protein may further comprise a signal sequence.

The “B-domain” of FVIII, as used herein, is the same as the B-domain known in the art that is defined by internal amino acid sequence identity and sites of proteolytic cleavage, e.g., residues Ser741-Arg1648 of full-length human FVIII. The other human FVIII domains are defined by the following amino acid residues: A1, residues Ala1-Arg372; A2, residues Ser373-Arg740; A3, residues Ser1690-Asn2019; C1, residues Lys2020-Asn2172; C2, residues Ser2173-Tyr2332. The A3-C1-C2 sequence includes residues Ser1690-Tyr2332. The remaining sequence, residues Glu1649-Arg1689, is usually referred to as the a3 acidic region. The locations of the boundaries for all of the domains, including the B-domains, for porcine, mouse and canine FVIII are also known in the art. In one embodiment, the B domain of FVIII is deleted (“B-domain-deleted factor VIII” or “BDD FVIII”). An example of a BDD FVIII is REFACTO® (recombinant BDD FVIII), which has the same sequence as the Factor VIII portion of the sequence in Table 5. (BDD FVIII heavy chain is double underlined; B domain is italicized; and BDD FVIII light chain is in plain text).

TABLE 5
BDD FVIII (SEQ ID NO: 18)

ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNPPVLK
RHQREITRTTLQSDQEEIDYDDTISVEMKKEDFDIYDEDENQSPRSFQKK
TRHYFIAAVERLWDYGMSSSPHVLRNRAQSGSVPQFKKVVFQEFTDGSFT
QPLYRGELNEHLGLLGPYIRAEVEDNIMVTFRNQASRPYSFYSSLISYEE
DQRQGAEPRKNFVKPNETKTYFWKVQHHMAPTKDEFDCKAWAYFSDVDLE
KDVHSGLIGPLLVCHTNTLNPAHGRQVTVQEFALFFTIFDETKSWYFTEN
MERNCRAPCNIQMEDPTFKENYRFHAINGYIMDTLPGLVMAQDQRIRWYL
LSMGSNENIHSIHFSGHVFTVRKKEEYKMALYNLYPGVFETVEMLPSKAG
IWRVECLIGEHLHAGMSTLFLVYSNKCQTPLGMASGHIRDFQITASGQYG
QWAPKLARLHYSGSINAWSTKEPFSWIKVDLLAPMIIHGIKTQGARQKFS
SLYISQFIIMYSLDGKKWQTYRGNSTGTLMVFFGNVDSSGIKHNIFNPPI
IARYIRLHPTHYSIRSTLRMELMGCDLNSCSMPLGMESKAISDAQITASS
YFTNMFATWSPSKARLHLQGRSNAWRPQVNNPKEWLQVDFQKTMKVTGVT
TQGVKSLLTSMYVKEFLISSSQDGHQWTLFFQNGKVKVFQGNQDSFTPVV
NSLDPPLLTRYLRIHPQSWVHQIALRMEVLGCEAQDLY

TABLE 6
Nucleotide Sequence Encoding BDD FVIII (SEQ ID NO: 19)*

| | |
|---|---|
| 661 | A TGCAAATAGA GCTCTCCACC TGCTTCTTTC |
| 721 | TGTGCCTTTT GCGATTCTGC TTTAGTGCCA CCAGAAGATA CTACCTGGGT GCAGTGGAAC |
| 781 | TGTCATGGGA CTATATGCAA AGTGATCTCG GTGAGCTGCC TGTGGACGCA AGATTTCCTC |
| 841 | CTAGAGTGCC AAAATCTTTT CCATTCAACA CCTCAGTCGT GTACAAAAAG ACTCTGTTTG |
| 901 | TAGAATTCAC GGATCACCTT TTCAACATCG CTAAGCCAAG GCCACCCTGG ATGGGTCTGC |
| 961 | TAGGTCCTAC CATCCAGGCT GAGGTTTATG ATACAGTGGT CATTACACTT AAGAACATGG |
| 1021 | CTTCCCATCC TGTCAGTCTT CATGCTGTTG GTGTATCCTA CTGGAAAGCT TCTGAGGGAG |
| 1081 | CTGAATATGA TGATCAGACC AGTCAAAGGG AGAAAGAAGA TGATAAAGTC TTCCCTGGTG |
| 1141 | GAAGCCATAC ATATGTCTGG CAGGTCCTGA AAGAGAATGG TCCAATGGCC TCTGACCCAC |
| 1201 | TGTGCCTTAC CTACTCATAT CTTTCTCATG TGGACCTGGT AAAAGACTTG AATTCAGGCC |
| 1261 | TCATTGGAGC CCTACTAGTA TGTAGAGAAG GGAGTCTGGC CAAGGAAAAG ACACAGACCT |
| 1321 | TGCACAAATT TATACTACTT TTTGCTGTAT TTGATGAAGG GAAAAGTTGG CACTCAGAAA |
| 1381 | CAAAGAACTC CTTGATGCAG GATAGGGATG CTGCATCTGC TCGGGCCTGG CCTAAAATGC |
| 1441 | ACACAGTCAA TGGTTATGTA AACAGGTCTC TGCCAGGTCT GATTGGATGC CACAGGAAAT |
| 1501 | CAGTCTATTG GCATGTGATT GGAATGGGCA CCACTCCTGA AGTGCACTCA ATATTCCTCG |
| 1561 | AAGGTCACAC ATTTCTTGTG AGGAACCATC GCCAGGCGTC CTTGGAAATC TCGCCAATAA |
| 1621 | CTTTCCTTAC TGCTCAAACA CTCTTGATGG ACCTTGGACA GTTTCTACTG TTTTGTCATA |
| 1681 | TCTCTTCCCA CCAACATGAT GGCATGGAAG CTTATGTCAA AGTAGACAGC TGTCCAGAGG |
| 1741 | AACCCCAACT ACGAATGAAA AATAATGAAG AAGCGGAAGA CTATGATGAT GATCTTACTG |
| 1601 | ATTCTGAAAT GGATGTGGTC AGGTTTGATG ATGACAACTC TCCTTCCTTT ATCCAAATTC |
| 1861 | GCTCAGTTGC CAAGAAGCAT CCTAAAACTT GGGTACATTA CATTGCTGCT GAAGAGGAGG |
| 1921 | ACTGGGACTA TGCTCCCTTA GTCCTCGCCC CCGATGACAG AAGTTATAAA AGTCAATATT |
| 1981 | TGAACAATGG CCCTCAGCGG ATTGGTAGGA AGTACAAAAA AGTCCGATTT ATGGCATACA |
| 2041 | CAGATGAAAC CTTTAAGACT CGTGAAGCTA TTCAGCATGA ATCAGGAATC TTGGGACCTT |
| 2101 | TACTTTATGG GGAAGTTGGA GACACACTGT TGATTATATT TAAGAATCAA GCAAGCAGAC |
| 2161 | CATATAACAT CTACCCTCAC GGAATCACTG ATGTCCGTCC TTTGTATTCA AGGAGATTAC |
| 2221 | CAAAAGGTGT AAAACATTTG AAGGATTTTC CAATTCTGCC AGGAGAAATA TTCAAATATA |
| 2281 | AATGGACAGT GACTGTAGAA GATGGGCCAA CTAAATCAGA TCCTCGGTGC CTGACCCGCT |
| 2341 | ATTACTCTAG TTTCGTTAAT ATGGAGAGAG ATCTAGCTTC AGGACTCATT GGCCCTCTCC |
| 2401 | TCATCTGCTA CAAAGAATCT GTAGATCAAA GAGGAAACCA GATAATGTCA GACAAGAGGA |
| 2461 | ATGTCATCCT GTTTTCTGTA TTTGATGAGA ACCGAAGCTG GTACCTCACA GAGAATATAC |
| 2521 | AACGCTTTCT CCCCAATCCA GCTGGAGTGC AGCTTGAGGA TCCAGAGTTC CAAGCCTCCA |
| 2581 | ACATCATGCA CAGCATCAAT GGCTATGTTT TTGATAGTTT GCAGTTGTCA GTTTGTTTGC |
| 2641 | ATGAGGTGGC ATACTGGTAC ATTCTAAGCA TTGGAGCACA GACTGACTTC CTTTCTGTCT |
| 2701 | TCTTCTCTGG ATATACCTTC AAACACAAAA TGGTCTATGA AGACACACTC ACCCTATTCC |
| 2761 | CATTCTCAGG AGAAACTGTC TTCATGTCGA TGGAAAACCC AGGTCTATGG ATTCTGGGGT |
| 2821 | GCCACAACTC AGACTTTCGG AACAGAGGCA TGACCGCCTT ACTGAAGGTT TCTAGTTGTG |
| 2881 | ACAAGAACAC TGGTGATTAT TACGAGGACA GTTATGAAGA TATTTCAGCA TACTTGCTGA |
| 2941 | GTAAAAACAA TGCCATTGAA CCAAGAAGCT TCTCTCAAAA CCCACCAGTC TTGAAACGCC |
| 3001 | ATCAACGGGA AATAACTCGT ACTACTCTTC AGTCAGATCA AGAGGAAATT GACTATGATG |
| 3061 | ATACCATATC AGTTGAAATG AAGAAGGAAG ATTTTGACAT TTATGATGAG GATGAAAATC |
| 3121 | AGAGCCCCCG CAGCTTTCAA AAGAAAACAC GACACTATTT TATTGCTGCA GTGGAGAGGC |
| 3181 | TCTGGGATTA TGGGATGAGT AGCTCCCCAC ATGTTCTAAG AAACAGGGCT CAGAGTGGCA |
| 3241 | GTGTCCCTCA GTTCAAGAAA GTTGTTTTCC AGGAATTTAC TGATGGCTCC TTTACTCAGC |
| 3301 | CCTTATACCG TGGAGAACTA AATGAACATT TGGGACTCCT GGGGCCATAT ATAAGAGCAG |
| 3361 | AAGTTGAAGA TAATATCATG GTAACTTTCA GAAATCAGGC CTCTCGTCCC TATTCCTTCT |
| 3421 | ATTCTAGCCT TATTTCTTAT GAGGAAGATC AGAGGCAAGG AGCAGAACCT AGAAAAAACT |
| 3481 | TTGTCAAGCC TAATGAAACC AAAACTTACT TTTGGAAAGT GCAACATCAT ATGGCACCCA |
| 3541 | CTAAAGATGA GTTTGACTGC AAAGCCTGGG CTTATTTCTC TGATGTTGAC CTGGAAAAAG |
| 3601 | ATGTGCACTC AGGCCTGATT GGACCCCTTC TGGTCTGCCA CACTAACACA CTGAACCCTG |
| 3661 | CTCATGGGAG ACAAGTGACA GTACAGGAAT TTGCTCTGTT TTTCACCATC TTTGATGAGA |
| 3721 | CCAAAAGCTG GTACTTCACT GAAAATATGG AAAGAAACTG CAGGGCTCCC TGCAATATCC |
| 3781 | AGATGGAAGA TCCCACTTTT AAAGAGAATT ATCGCTTCCA TGCAATCAAT GGCTACATAA |
| 3841 | TGGATACACT ACCTGGCTTA GTAATGGCTC AGGATCAAAG GATTCGATGG TATCTGCTCA |
| 3901 | GCATGGGCAG CAATGAAAAC ATCCATTCTA TTCATTTCAG TGGACATGTG TTCACTGTAC |
| 3961 | GAAAAAAAGA GGAGTATAAA ATGGCACTGT ACAATCTCTA TCCAGGTGTT TTTGAGACAG |
| 4021 | TGGAAATGTT ACCATCCAAA GCTGGAATTT GGCGGGTGGA ATGCCTTATT GGCGAGCATC |
| 4081 | TACATGCTGG GATGAGCACA CTTTTTCTGG TGTACAGCAA TAAGTGTCAG ACTCCCCTGG |
| 4141 | GAATGGCTTC TGGACACATT AGAGATTTTC AGATTACAGC TTCAGGACAA TATGGACAGT |
| 4201 | GGGCCCCAAA GCTGGCCAGA CTTCATTATT CCGGATCAAT CAATGCCTGG AGCACCAAGG |
| 4261 | AGCCCTTTTC TTGGATCAAG GTGGATCTGT TGGCACCAAT GATTATTCAC GGCATCAAGA |
| 4321 | CCCAGGGTGC CCGTCAGAAG TTCTCCAGCC TCTACATCTC TCAGTTTATC ATCATGTATA |
| 4381 | GTCTTGATGG GAAGAAGTGG CAGACTTATC GAGGAAATTC CACTGGAACC TTAATGGTCT |
| 4441 | TCTTTGGCAA TGTGGATTCA TCTGGGATAA AACACAATAT TTTTAACCCT CCAATTATTG |
| 4501 | CTCGATACAT CCGTTTGCAC CCAACTCATT ATAGCATTCG CAGCACTCTT CGCATGGAGT |
| 4561 | TGATGGGCTG TGATTTAAAT AGTTGCAGCA TGCCATTGGG AATGGAGAGT AAAGCAATAT |
| 4621 | CAGATGCACA GATTACTGCT TCATCCTACT TTACCAATAT GTTTGCCACC TGGTCTCCTT |
| 4681 | CAAAAGCTCG ACTTCACCTC CAAGGGAGGA GTAATGCCTG GAGACCTCAG GTGAATAATC |
| 4741 | CAAAAGAGTG GCTGCAAGTG GACTTCCAGA AGACAATGAA AGTCACAGGA GTAACTACTC |
| 4801 | AGGGAGTAAA ATCTCTGCTT ACCAGCATGT ATGTGAAGGA GTTCCTCATC TCCAGCAGTC |
| 4861 | AAGATGGCCA TCAGTGGACT CTCTTTTTTC AGAATGGCAA AGTAAAGGTT TTTCAGGGAA |
| 4921 | ATCAAGACTC CTTCACACCT GTGGTGAACT CTCTAGACCC ACCGTTACTG ACTCGCTACC |
| 4981 | TTCGAATTCA CCCCCAGAGT TGGGTGCACC AGATTGCCCT GAGGATGGAG GTTCTGGGCT |
| 5041 | GCGAGGCACA GGACCTCTAC |

*The underlined nucleic acids encode a signal peptide.

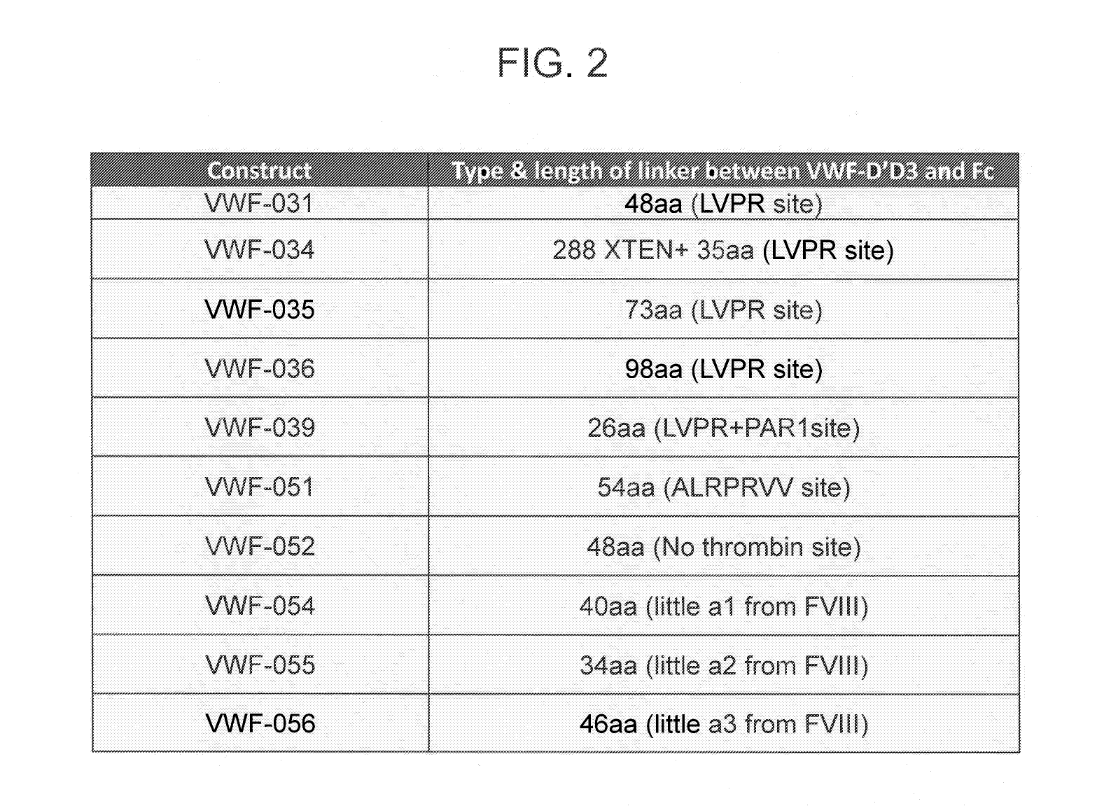

A “B-domain-deleted FVIII” may have the full or partial deletions disclosed in U.S. Pat. Nos. 6,316,226, 6,346,513, 7,041,635, 5,789,203, 6,060,447, 5,595,886, 6,228,620, 5,972,885, 6,048,720, 5,543,502, 5,610,278, 5,171,844, 5,112,950, 4,868,112, and 6,458,563. In some embodiments, a B-domain-deleted FVIII sequence of the present invention comprises any one of the deletions disclosed at col. 4, line 4 to col. 5, line 28 and Examples 1-5 of U.S. Pat. No. 6,316,226 (also in U.S. Pat. No. 6,346,513). In another embodiment, a B-domain deleted Factor VIII is the S743/Q1638 B-domain deleted Factor VIII (SQ BDD FVIII) (e.g., Factor VIII having a deletion from amino acid 744 to amino acid 1637, e.g., Factor VIII having amino acids 1-743 and amino acids 1638-2332 of SEQ ID NO: 16, i.e., SEQ ID NO: 18). In some embodiments, a B-domain-deleted FVIII of the present invention has a deletion disclosed at col. 2, lines 26-51 and examples 5-8 of U.S. Pat. No. 5,789,203 (also U.S. Pat. No. 6,060,447, U.S. Pat. No. 5,595,886, and U.S. Pat. No. 6,228,620). In some embodiments, a B-domain-deleted Factor VIII has a deletion described in col. 1, lines 25 to col. 2, line 40 of U.S. Pat. No. 5,972,885; col. 6, lines 1-22 and example 1 of U.S. Pat. No. 6,048,720; col. 2, lines 17-46 of U.S. Pat. No. 5,543,502; col. 4, line 22 to col. 5, line 36 of U.S. Pat. No. 5,171,844; col. 2, lines 55-68, FIG. 2, and example 1 of U.S. Pat. No. 5,112,950; col. 2, line 2 to col. 19, line 21 and table 2 of U.S. Pat. No. 4,868,112; col. 2, line 1 to col. 3, line 19, col. 3, line 40 to col. 4, line 67, col. 7, line 43 to col. 8, line 26, and col. 11, line 5 to col. 13, line 39 of U.S. Pat. No. 7,041,635; or col. 4, lines 25-53, of U.S. Pat. No. 6,458,563.

In some embodiments, a B-domain-deleted FVIII has a deletion of most of the B domain, but still contains amino-terminal sequences of the B domain that are essential for in vivo proteolytic processing of the primary translation product into two polypeptide chain, as disclosed in WO 91/09122. In some embodiments, a B-domain-deleted FVIII is constructed with a deletion of amino acids 747-1638, i.e., virtually a complete deletion of the B domain. Hoeben R. C., et al. J. Biol. Chem. 265 (13): 7318-7323 (1990). A B-domain-deleted Factor VIII may also contain a deletion of amino acids 771-1666 or amino acids 868-1562 of FVIII. Meulien P., et al. Protein Eng. 2(4): 301-6 (1988). Additional B domain deletions that are part of the invention include: deletion of amino acids 982 through 1562 or 760 through 1639 (Toole et al., Proc. Natl. Acad. Sci. U.S.A. (1986) 83, 5939-5942)), 797 through 1562 (Eaton, et al. Biochemistry (1986) 25:8343-8347)), 741 through 1646 (Kaufman (PCT published application No. WO 87/04187)), 747-1560 (Sarver, et al., DNA (1987) 6:553-564)), 741 through 1648 (Pasek (PCT application No. 88/00831)), or 816 through 1598 or 741 through 1648 (Lagner (Behring Inst. Mitt. (1988) No 82:16-25, EP 295597)). In other embodiments, BDD FVIII includes a FVIII polypeptide containing fragments of the B-domain that retain one or more N-linked glycosylation sites, e.g., residues 757, 784, 828, 900, 963, or optionally 943, which correspond to the amino acid sequence of the full-length FVIII sequence. Examples of the B-domain fragments include 226 amino acids or 163 amino acids of the B-domain as disclosed in Miao, H. Z., et al., Blood 103(a): 3412-3419 (2004), Kasuda, A, et al., J. Thromb. Haemost. 6: 1352-1359 (2008), and Pipe, S. W., et al., J. Thromb. Haemost. 9: 2235-2242 (2011) (i.e., the first 226 amino acids or 163 amino acids of the B domain are retained). In some embodiments, the FVIII with a partial B-domain is FVIII198. FVIII198 is a partial B-domain containing single chain FVIIIFc molecule-226N6. 226 represents the N-terminus 226 amino acid of the FVIII B-domain, and N6 represents six N-glycosylation sites in the B-domain. In still other embodiments, BDD FVIII further comprises a point mutation at residue 309 (from Phe to Ser) to improve expression of the BDD FVIII protein. See Miao, H. Z., et al., Blood 103(a): 3412-3419 (2004). In still other embodiments, the BDD FVIII includes a FVIII polypeptide containing a portion of the B-domain, but not containing one or more furin cleavage sites (e.g., Arg1313 and Arg 1648). See Pipe, S. W., et al., J. Thromb. Haemost. 9: 2235-2242 (2011). Each of the foregoing deletions may be made in any FVIII sequence.

A FVIII protein useful in the present invention can include FVIII having one or more additional heterologous sequences or chemical or physical modifications therein, which do not affect the FVIII coagulation activity. Such heterologous sequences or chemical or physical modifications can be fused to the C-terminus or N-terminus of the FVIII protein or inserted between one or more of the two amino acid residues in the FVIII protein. Such insertions in the FVIII protein do not affect the FVIII coagulation activity or FVIII function. In one embodiment, the insertions improve pharmacokinetic properties of the FVIII protein (e.g., half-life). In another embodiment, the insertions can be more than two, three, four, five, or six sites.

In one embodiment, FVIII is cleaved right after Arginine at amino acid 1648 (in full-length Factor VIII or SEQ ID NO: 16), amino acid 754 (in the S743/Q1638 B-domain deleted Factor VIII or SEQ ID NO: 16), or the corresponding Arginine residue (in other variants), thereby resulting in a heavy chain and a light chain. In another embodiment, FVIII comprises a heavy chain and a light chain, which are linked or associated by a metal ion-mediated non-covalent bond.

In other embodiments, FVIII is a single chain FVIII that has not been cleaved right after Arginine at amino acid 1648 (in full-length FVIII or SEQ ID NO: 16), amino acid 754 (in the S743/Q1638 B-domain-deleted FVIII or SEQ ID NO: 18), or the corresponding Arginine residue (in other variants). A single chain FVIII may comprise one or more amino acid substitutions. In one embodiment, the amino acid substitution is at a residue corresponding to residue 1648, residue 1645, or both of full-length mature Factor VIII polypeptide (SEQ ID NO: 16) or residue 754, residue 751, or both of SQ BDD Factor VIII (SEQ ID NO: 18). The amino acid substitution can be any amino acids other than arginine, e.g., isoleucine, leucine, lysine, methionine, phenylalanine, threonine, tryptophan, valine, alanine, asparagine, aspartic acid, cysteine, glutamic acid, glutamine, glycine, proline, selenocysteine, serine, tyrosine, histidine, ornithine, pyrrolysine, or taurine.

FVIII can further be cleaved by thrombin and then activated as FVIIIa, serving as a cofactor for activated Factor IX (FIXa). And the activated FIX together with activated FVIII forms a Xase complex and converts Factor X to activated Factor X (FXa). For activation, FVIII is cleaved by thrombin after three Arginine residues, at amino acids 372, 740, and 1689 (corresponding to amino acids 372, 740, and 795 in the B-domain deleted FVIII sequence), the cleavage generating FVIIIa having the 50 kDa A1, 43 kDa A2, and 73 kDa A3-C1-C2 chains. In one embodiment, the FVIII protein useful for the present invention is non-active FVIII. In another embodiment, the FVIII protein is an activated FVIII.

The protein having FVIII polypeptide linked to or associated with the VWF protein can comprise a sequence at least 50%, 60%, 70%, 80%, 90%, 95%, 96%, 97%, 98%, 99%, or 100% identical to SEQ ID NO: 16 or 18, wherein the sequence has the FVIII clotting activity, e.g., activating Factor IX as a cofactor to convert Factor X to activated Factor X (FXa).

In some embodiments, the FVIII protein further comprises one or more heterologous moieties that are fused to the C-terminus or N-terminus of the FVIII protein or that are inserted between two adjacent amino acids in the FVIII protein. In other embodiments, the heterologous moieties comprise an amino acid sequence of at least about 50 amino acids, at least about 100 amino acids, at least about 150 amino acids, at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, at least about 850 amino acids, at least about 900 amino acids, at least about 950 amino acids, or at least about 1000 amino acids. In some embodiments, the half-life of the chimeric molecule is extended at least about 1.5 times, at least about 2 times, at least about 2.5 times, at least about 3 times, at least about 4 times, at least about 5 times, at least about 6 times, at least about 7 times, at least about 8 times, at least about 9 times, at least about 10 times, at least about 11 times, or at least about 12 times longer than wild-type FVIII.

Other exemplary FVIII variants are also disclosed in US Publication No. US2013/0017997, published Jan. 17, 2013, International Publication No. WO 2013/122617, published Aug. 22, 2013, or International Publication No. WO 2014/011819, published Jan. 16, 2014, or International Publication No. WO2013123457 A1, or International Application No. PCT/US2013/049989.

### III. POLYNUCLEOTIDES, VECTORS, HOST CELLS, AND METHODS OF MAKING

Also provided in the invention is a polynucleotide encoding the chimeric molecule described herein. When a VWF protein is linked to a heterologous moiety via a VWF linker and to a FVIII protein and an XTEN sequence in a chimeric protein as a single polypeptide chain, the invention is drawn to a single polynucleotide encoding the single polypeptide chain. When the chimeric protein comprises a first and a second polypeptide chains, the first polypeptide chain comprising a VWF protein, an XTEN sequence, and a first heterologous moiety (e.g., a first Fc region) via a VWF linker and the second polypeptide chain comprising a FVIII protein and a second heterologous moiety (e.g., a second Fc region), a polynucleotide can comprise the first nucleotide region and the second nucleotide region. In one embodiment, the first nucleotide region and the second nucleotide region are on the same polynucleotide. In another embodiment, the first nucleotide region and the second nucleotide region are on two different polynucleotides (e.g., different vectors). In certain embodiments, the present invention is directed to a set of polynucleotides comprising a first nucleotide chain and a second nucleotide chain, wherein the first nucleotide chain encodes a VWF protein, an XTEN sequence, a VWF linker, and a heterologous moiety of the chimeric protein and the second nucleotide chain encodes a FVIII protein and a second heterologous moiety. In some embodiments, the present invention is directed to a set of polynucleotides comprising a first nucleotide chain and a second nucleotide chain, wherein the first nucleotide chain encodes a VWF protein, and a heterologous moiety of the chimeric protein and the second nucleotide chain encodes a FVIII protein fused to a second heterologous moiety via a FVIII linker, wherein at least one XTEN sequence is fused to the chimeric protein. In other embodiments, the present invention is directed to a set of polynucleotides comprising a first nucleotide chain and a second nucleotide chain, wherein the first nucleotide chain encodes a VWF protein, a VWF linker, and a heterologous moiety of the chimeric protein and the second nucleotide chain encodes a FVIII protein, a FVIII linker, and a second heterologous moiety, wherein at least one XTEN sequence is fused to the chimeric protein.

In other embodiments, the set of polynucleotides further comprises an additional nucleotide chain (e.g., a second nucleotide chain when the chimeric polypeptide is encoded by a single polynucleotide chain or a third nucleotide chain when the chimeric protein is encoded by two polynucleotide chains) which encodes a protein convertase. The protein convertase can be selected from proprotein convertase subtilisin/kexin type 5 (PCSK5 or PC5), proprotein convertase subtilisin/kexin type 7 (PCSK7 or PC5), a yeast Kex 2, proprotein convertase subtilisin/kexin type 3 (PACE or PCSK3), or two or more combinations thereof. In some embodiments, the protein convertase is PACE, PC5, or PC7. In a specific embodiment, the protein convertase is PC5 or PC7. See International Application no. PCT/US2011/043568, which is incorporated herein by reference. In another embodiment, the protein convertase is PACE/furin.

In certain embodiments, the invention includes a set of the polynucleotides comprising a first nucleotide sequence encoding a VWF protein comprising a D′ domain and a D3 domain of VWF fused to a first heterologous moiety via a VWF linker, a second nucleotide sequence encoding a FVIII protein and a second heterologous moiety, and a third nucleotide sequence encoding a D1 domain and D2 domain of VWF and wherein an XTEN sequence is present either in the first chain or in the second chain. In this embodiment, the D1 domain and D2 domain are separately expressed (not linked to the D′D3 domain of the VWF protein) in order for the proper disulfide bond formation and folding of the D′D3 domains. The D1D2 domain expression can either be in cis or trans.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

Expression vectors of the invention will include polynucleotides encoding the chimeric molecule.

In one embodiment, a coding sequence for the chimeric molecule is operably linked to an expression control sequence. As used herein, two nucleic acid sequences are operably linked when they are covalently linked in such a way as to permit each component nucleic acid sequence to retain its functionality. A coding sequence and a gene expression control sequence are said to be operably linked when they are covalently linked in such a way as to place the expression or transcription and/or translation of the coding sequence under the influence or control of the gene expression control sequence. Two DNA sequences are said to be operably linked if induction of a promoter in the 5′ gene expression sequence results in the transcription of the coding sequence and if the nature of the linkage between the two DNA sequences does not (1) result in the introduction of a frame-shift mutation, (2) interfere with the ability of the promoter region to direct the transcription of the coding sequence, or (3) interfere with the ability of the corresponding RNA transcript to be translated into a protein. Thus, a gene expression sequence would be operably linked to a coding nucleic acid sequence if the gene expression sequence were capable of effecting transcription of that coding nucleic acid sequence such that the resulting transcript is translated into the desired protein or polypeptide.

A gene expression control sequence as used herein is any regulatory nucleotide sequence, such as a promoter sequence or promoter-enhancer combination, which facilitates the efficient transcription and translation of the coding nucleic acid to which it is operably linked. The gene expression control sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression control sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined coding nucleic acid. The gene expression sequences optionally include enhancer sequences or upstream activator sequences as desired.

Viral vectors include, but are not limited to, nucleic acid sequences from the following viruses: retrovirus, such as Moloney murine leukemia virus, Harvey murine sarcoma virus, murine mammary tumor virus, and Rous sarcoma virus; adenovirus, adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., Gene Transfer and Expression. A Laboratory Manual, W.H. Freeman Co., New York (1990) and Murry, E. J., Methods in Molecular Biology, Vol. 7, Humana Press, Inc., Cliffton, N.J. (1991).

In one embodiment, the virus is an adeno-associated virus, a double-stranded DNA virus. The adeno-associated virus can be engineered to be replication-deficient and is capable of infecting a wide range of cell types and species. It further has advantages such as heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, Calif.). Other plasmids are well-known to those of ordinary skill in the art. Additionally, plasmids may be custom designed using standard molecular biology techniques to remove and/or add specific fragments of DNA.

In one insect expression system that may be used to produce the proteins of the invention, Autographa californica nuclear polyhidrosis virus (AcNPV) is used as a vector to express the foreign genes. The virus grows in Spodoptera frugiperda cells. A coding sequence may be cloned into non-essential regions (for example, the polyhedron gene) of the virus and placed under control of an ACNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al. (1983) J Virol 46:584; U.S. Pat. No. 4,215,051). Further examples of this expression system may be found in Ausubel et al., eds. (1989) Current Protocols in Molecular Biology, Vol. 2, Greene Publish. Assoc. & Wiley Interscience.

Another system which can be used to express the proteins of the invention is the glutamine synthetase gene expression system, also referred to as the “GS expression system” (Lonza Biologics PLC, Berkshire UK). This expression system is described in detail in U.S. Pat. No. 5,981,216.

In mammalian host cells, a number of viral based expression systems may be utilized. In cases where an adenovirus is used as an expression vector, a coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing peptide in infected hosts. See, e.g., Logan & Shenk (1984) Proc Natl Acad Sci USA 81:3655). Alternatively, the vaccinia 7.5 K promoter may be used. See, e.g., Mackett et al. (1982) Proc Natl Acad Sci USA 79:7415; Mackett et al. (1984) J Virol 49:857; Panicali et al. (1982) Proc Natl Acad Sci USA 79:4927.

To increase efficiency of production, the polynucleotides can be designed to encode multiple units of the protein of the invention separated by enzymatic cleavage sites. The resulting polypeptide can be cleaved (e.g., by treatment with the appropriate enzyme) in order to recover the polypeptide units. This can increase the yield of polypeptides driven by a single promoter. When used in appropriate viral expression systems, the translation of each polypeptide encoded by the mRNA is directed internally in the transcript; e.g., by an internal ribosome entry site, IRES. Thus, the polycistronic construct directs the transcription of a single, large polycistronic mRNA which, in turn, directs the translation of multiple, individual polypeptides. This approach eliminates the production and enzymatic processing of polyproteins and may significantly increase the yield of polypeptides driven by a single promoter.

Vectors used in transformation will usually contain a selectable marker used to identify transformants. In bacterial systems, this can include an antibiotic resistance gene such as ampicillin or kanamycin. Selectable markers for use in cultured mammalian cells include genes that confer resistance to drugs, such as neomycin, hygromycin, and methotrexate. The selectable marker may be an amplifiable selectable marker. One amplifiable selectable marker is the dihydrofolate reductase (DHFR) gene. Simonsen C C et al. (1983) Proc Natl Acad Sci USA 80:2495-9. Selectable markers are reviewed by Thilly (1986) Mammalian Cell Technology, Butterworth Publishers, Stoneham, Mass., and the choice of selectable markers is well within the level of ordinary skill in the art.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, U.S. Pat. No. 4,713,339).

The expression vectors can encode for tags that permit easy purification of the recombinantly produced protein. Examples include, but are not limited to, vector pUR278 (Ruther et al. (1983) EMBO J 2:1791), in which coding sequences for the protein to be expressed may be ligated into the vector in frame with the lac z coding region so that a tagged fusion protein is produced; pGEX vectors may be used to express proteins of the invention with a glutathione S-transferase (GST) tag. These proteins are usually soluble and can easily be purified from cells by adsorption to glutathione-agarose beads followed by elution in the presence of free glutathione. The vectors include cleavage sites (thrombin or Factor Xa protease or PRESCISSION PROTEASE™ (Pharmacia, Peapack, N.J.)) for easy removal of the tag after purification.

The expression vector or vectors are then transfected or co-transfected into a suitable target cell, which will express the polypeptides. Transfection techniques known in the art include, but are not limited to, calcium phosphate precipitation (Wigler et al. (1978) Cell 14:725), electroporation (Neumann et al. (1982) EMBO J 1:841), and liposome-based reagents. A variety of host-expression vector systems may be utilized to express the proteins described herein including both prokaryotic and eukaryotic cells. These include, but are not limited to, microorganisms such as bacteria (e.g., E. coli) transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems, including mammalian cells (e.g., HEK 293, CHO, Cos, HeLa, HKB11, and BHK cells).

In one embodiment, the host cell is a eukaryotic cell. As used herein, a eukaryotic cell refers to any animal or plant cell having a definitive nucleus. Eukaryotic cells of animals include cells of vertebrates, e.g., mammals, and cells of invertebrates, e.g., insects. Eukaryotic cells of plants specifically can include, without limitation, yeast cells. A eukaryotic cell is distinct from a prokaryotic cell, e.g., bacteria.

In certain embodiments, the eukaryotic cell is a mammalian cell. A mammalian cell is any cell derived from a mammal. Mammalian cells specifically include, but are not limited to, mammalian cell lines. In one embodiment, the mammalian cell is a human cell. In another embodiment, the mammalian cell is a HEK 293 cell, which is a human embryonic kidney cell line. HEK 293 cells are available as CRL-1533 from American Type Culture Collection, Manassas, Va., and as 293-H cells, Catalog No. 11631-017 or 293-F cells, Catalog No. 11625-019 from Invitrogen (Carlsbad, Calif.). In some embodiments, the mammalian cell is a PER.C6® cell, which is a human cell line derived from retina. PER.C6® cells are available from Crucell (Leiden, The Netherlands). In other embodiments, the mammalian cell is a Chinese hamster ovary (CHO) cell. CHO cells are available from American Type Culture Collection, Manassas, Va. (e.g., CHO-K1; CCL-61). In still other embodiments, the mammalian cell is a baby hamster kidney (BHK) cell. BHK cells are available from American Type Culture Collection, Manassas, Va. (e.g., CRL-1632). In some embodiments, the mammalian cell is a HKB11 cell, which is a hybrid cell line of a HEK293 cell and a human B cell line. Mei et al., Mol. Biotechnol. 34(2): 165-78 (2006).

In one embodiment, a plasmid encoding a VWF protein, a VWF linker, a heterologous moiety or the chimeric protein of the invention further includes a selectable marker, e.g., zeocin resistance, and is transfected into HEK 293 cells, for production of the chimeric protein.

In still other embodiments, transfected cells are stably transfected. These cells can be selected and maintained as a stable cell line, using conventional techniques known to those of skill in the art.

Host cells containing DNA constructs of the protein are grown in an appropriate growth medium. As used herein, the term “appropriate growth medium” means a medium containing nutrients required for the growth of cells. Nutrients required for cell growth may include a carbon source, a nitrogen source, essential amino acids, vitamins, minerals, and growth factors. Optionally, the media can contain one or more selection factors. Optionally the media can contain bovine calf serum or fetal calf serum (FCS). In one embodiment, the media contains substantially no IgG. The growth medium will generally select for cells containing the DNA construct by, for example, drug selection or deficiency in an essential nutrient which is complemented by the selectable marker on the DNA construct or co-transfected with the DNA construct. Cultured mammalian cells are generally grown in commercially available serum-containing or serum-free media (e.g., MEM, DMEM, DMEM/F12). In one embodiment, the medium is CD293 (Invitrogen, Carlsbad, Calif.). In another embodiment, the medium is CD17 (Invitrogen, Carlsbad, Calif.). Selection of a medium appropriate for the particular cell line used is within the level of those ordinary skilled in the art.

In order to co-express two polypeptide echains of the chimeric molecule as described herein, the host cells are cultured under conditions that allow expression of both chains. As used herein, culturing refers to maintaining living cells in vitro for at least a definite time. Maintaining can, but need not include, an increase in population of living cells. For example, cells maintained in culture can be static in population, but still viable and capable of producing a desired product, e.g., a recombinant protein or recombinant fusion protein. Suitable conditions for culturing eukaryotic cells are well known in the art and include appropriate selection of culture media, media supplements, temperature, pH, oxygen saturation, and the like. For commercial purposes, culturing can include the use of any of various types of scale-up systems including shaker flasks, roller bottles, hollow fiber bioreactors, stirred-tank bioreactors, airlift bioreactors, Wave bioreactors, and others.

The cell culture conditions are also selected to allow association of the first chain and the second chain in the chimeric molecule. Conditions that allow expression of the chimeric molecule may include the presence of a source of vitamin K. For example, in one embodiment, stably transfected HEK 293 cells are cultured in CD293 media (Invitrogen, Carlsbad, Calif.) or OptiCHO media (Invitrogen, Carlsbad, Calif.) supplemented with 4 mM glutamine.

In one aspect, the present invention is directed to a method of expressing, making, or producing the chimeric protein comprising a) transfecting a host cell with a polynucleotide encoding the chimeric molecule and b) culturing the host cell in a culture medium under a condition suitable for expressing the chimeric molecule, wherein the chimeric molecule is expressed.

In further embodiments, the protein product containing the chimeric molecule is secreted into the media. Media is separated from the cells, concentrated, filtered, and then passed over two or three affinity columns, e.g., a protein A column and one or two anion exchange columns.

In certain aspects, the present invention relates to the chimeric polypeptide produced by the methods described herein.

In vitro production allows scale-up to give large amounts of the desired altered polypeptides of the invention. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, hydrophobic interaction chromatography (HIC, chromatography over DEAE-cellulose or affinity chromatography.

The invention also includes a method of improving FVIII activity of a chimeric FVIII protein comprising a VWF protein fused to a first heterologous moiety and an XTEN sequence and a FVIII protein fused to a second heterologous moiety, the method comprising inserting a VWF linker between the VWF protein and the first heterologous moiety, wherein the VWF linker comprises a polypeptide selected from: (i) an a2 region from Factor VIII (FVIII); (ii) an a1 region from FVIII; (iii) an a3 region from FVIII; (iv) a thrombin cleavage site which comprises X-V-P-R (SEQ ID NO: 3) and a PAR1 exosite interaction motif, wherein X is an aliphatic amino acid; or (v) any combination thereof. In some embodiments, the FVIII activity is measured by aPTT assay or ROTEM assay.

### IV. PHARMACEUTICAL COMPOSITION

Compositions containing the chimeric molecule of the present invention may contain a suitable pharmaceutically acceptable carrier. For example, they may contain excipients and/or auxiliaries that facilitate processing of the active compounds into preparations designed for delivery to the site of action.

The pharmaceutical composition can be formulated for parenteral administration (i.e., intravenous, subcutaneous, or intramuscular) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, e.g., pyrogen free water.

Suitable formulations for parenteral administration also include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances, which increase the viscosity of the suspension, including, for example, sodium carboxymethyl cellulose, sorbitol and dextran. Optionally, the suspension may also contain stabilizers. Liposomes also can be used to encapsulate the molecules of the invention for delivery into cells or interstitial spaces. Exemplary pharmaceutically acceptable carriers are physiologically compatible solvents, dispersion media, coatings, antibacterial and antifungal agents, isotonic and absorption delaying agents, water, saline, phosphate buffered saline, dextrose, glycerol, ethanol and the like. In some embodiments, the composition comprises isotonic agents, for example, sugars, polyalcohols such as mannitol, sorbitol, or sodium chloride. In other embodiments, the compositions comprise pharmaceutically acceptable substances such as wetting agents or minor amounts of auxiliary substances such as wetting or emulsifying agents, preservatives or buffers, which enhance the shelf life or effectiveness of the active ingredients.

Compositions of the invention may be in a variety of forms, including, for example, liquid (e.g., injectable and infusible solutions), dispersions, suspensions, semi-solid and solid dosage forms. The preferred form depends on the mode of administration and therapeutic application.

The composition can be formulated as a solution, micro emulsion, dispersion, liposome, or other ordered structure suitable to high drug concentration. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The active ingredient can be formulated with a controlled-release formulation or device. Examples of such formulations and devices include implants, transdermal patches, and microencapsulated delivery systems. Biodegradable, biocompatible polymers can be used, for example, ethylene vinyl acetate, polyanhydrides, polyglycolic acid, collagen, polyorthoesters, and polylactic acid. Methods for the preparation of such formulations and devices are known in the art. See e.g., Sustained and Controlled Release Drug Delivery Systems, J. R. Robinson, ed., Marcel Dekker, Inc., New York, 1978.

Injectable depot formulations can be made by forming microencapsulated matrices of the drug in biodegradable polymers such as polylactide-polyglycolide. Depending on the ratio of drug to polymer, and the nature of the polymer employed, the rate of drug release can be controlled. Other exemplary biodegradable polymers are polyorthoesters and polyanhydrides. Depot injectable formulations also can be prepared by entrapping the drug in liposomes or microemulsions.

Supplementary active compounds can be incorporated into the compositions. In one embodiment, a chimeric molecule of the invention is formulated with another clotting factor, or a variant, fragment, analogue, or derivative thereof. For example, the clotting factor includes, but is not limited to, factor V, factor VII, factor VIII, factor IX, factor X, factor XI, factor XII, factor XIII, prothrombin, fibrinogen, von Willebrand factor or recombinant soluble tissue factor (rsTF) or activated forms of any of the preceding. The clotting factor of hemostatic agent can also include anti-fibrinolytic drugs, e.g., epsilon-amino-caproic acid, tranexamic acid.

Dosage regimens may be adjusted to provide the optimum desired response. For example, a single bolus may be administered, several divided doses may be administered over time, or the dose may be proportionally reduced or increased as indicated by the exigencies of the therapeutic situation. It is advantageous to formulate parenteral compositions in dosage unit form for ease of administration and uniformity of dosage. See, e.g., Remington's Pharmaceutical Sciences (Mack Pub. Co., Easton, Pa. 1980).

In addition to the active compound, the liquid dosage form may contain inert ingredients such as water, ethyl alcohol, ethyl carbonate, ethyl acetate, benzyl alcohol, benzyl benzoate, propylene glycol, 1,3-butylene glycol, dimethylformamide, oils, glycerol, tetrahydrofurfuryl alcohol, polyethylene glycols, and fatty acid esters of sorbitan.

Non-limiting examples of suitable pharmaceutical carriers are also described in Remington's Pharmaceutical Sciences by E. W. Martin. Some examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

For oral administration, the pharmaceutical composition can take the form of tablets or capsules prepared by conventional means. The composition can also be prepared as a liquid for example a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal administration, the composition may take the form of tablets or lozenges according to conventional protocols.

For administration by inhalation, the compounds for use according to the present invention are conveniently delivered in the form of a nebulized aerosol with or without excipients or in the form of an aerosol spray from a pressurized pack or nebulizer, with optionally a propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can also be formulated for rectal administration as a suppository or retention enema, e.g., containing conventional suppository bases such as cocoa butter or other glycerides.

### V. GENE THERAPY

A chimeric molecule of the invention can be produced in vivo in a mammal, e.g., a human patient, using a gene therapy approach to treatment of a bleeding disease or disorder selected from a bleeding coagulation disorder, hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, or bleeding in the illiopsoas sheath would be therapeutically beneficial. In one embodiment, the bleeding disease or disorder is hemophilia. In another embodiment, the bleeding disease or disorder is hemophilia A. This involves administration of a suitable chimeric molecule-encoding nucleic acid operably linked to suitable expression control sequences. In certain embodiment, these sequences are incorporated into a viral vector. Suitable viral vectors for such gene therapy include adenoviral vectors, lentiviral vectors, baculoviral vectors, Epstein Barr viral vectors, papovaviral vectors, vaccinia viral vectors, herpes simplex viral vectors, and adeno associated virus (AAV) vectors. The viral vector can be a replication-defective viral vector. In other embodiments, a adenoviral vector has a deletion in its E1 gene or E3 gene. When an adenoviral vector is used, the mammal may not be exposed to a nucleic acid encoding a selectable marker gene. In other embodiments, the sequences are incorporated into a non-viral vector known to those skilled in the art.

### VI. METHODS OF USING CHIMERIC PROTEIN

The present invention further provides a method for reducing a frequency or degree of a bleeding episode in a subject in need thereof using a chimeric molecule of the invention. An exemplary method comprises administering to the subject in need thereof a therapeutically effective amount of a chimeric molecule of the invention. In other aspects, the invention includes a method of preventing an occurrence of a bleeding episode in a subject in need thereof using a chimeric molecule of the invention. In other aspects, composition comprising a DNA encoding the recombinant protein of the invention can be administered to a subject in need thereof. In certain aspects of the invention, a cell expressing a chimeric molecule of the invention can be administered to a subject in need thereof. In certain aspects of the invention, the pharmaceutical composition comprises (i) a chimeric molecule, (ii) an isolated nucleic acid encoding a chimeric molecule, (iii) a vector comprising a nucleic acid encoding a chimeric molecule, (iv) a cell comprising an isolated nucleic acid encoding a chimeric molecule and/or a vector comprising a nucleic encoding a chimeric molecule, or (v) a combination thereof, and the pharmaceutical compositions further comprises an acceptable excipient or carrier.

The bleeding episode can be caused by or derived from a blood coagulation disorder. A blood coagulation disorder can also be referred to as a coagulopathy. In one example, the blood coagulation disorder, which can be treated with a pharmaceutical composition of the current disclosure, is hemophilia or von Willebrand disease (vWD). In another example, the blood coagulation disorder, which can be treated with a pharmaceutical composition of the present disclosure is hemophilia A.

In some embodiments, the type of bleeding associated with the bleeding condition is selected from hemarthrosis, muscle bleed, oral bleed, hemorrhage, hemorrhage into muscles, oral hemorrhage, trauma, trauma capitis, gastrointestinal bleeding, intracranial hemorrhage, intra-abdominal hemorrhage, intrathoracic hemorrhage, bone fracture, central nervous system bleeding, bleeding in the retropharyngeal space, bleeding in the retroperitoneal space, bleeding in the illiopsoas sheath, or any combination thereof.

In other embodiments, the subject suffering from bleeding condition is in need of treatment for surgery, including, e.g., surgical prophylaxis or peri-operative management. In one example, the surgery is selected from minor surgery and major surgery. Exemplary surgical procedures include tooth extraction, tonsillectomy, inguinal herniotomy, synovectomy, craniotomy, osteosynthesis, trauma surgery, intracranial surgery, intra-abdominal surgery, intrathoracic surgery, joint replacement surgery (e.g., total knee replacement, hip replacement, and the like), heart surgery, and caesarean section.

In another example, the subject is concomitantly treated with Factor IX. Because the compounds of the invention are capable of activating FIXa, they could be used to pre-activate the FIXa polypeptide before administration of the FIXa to the subject.

The methods of the invention may be practiced on a subject in need of prophylactic treatment or on-demand treatment.

Pharmaceutical compositions comprising a chimeric molecule of the invention may be formulated for any appropriate manner of administration, including, for example, topical (e.g., transdermal or ocular), oral, buccal, nasal, vaginal, rectal or parenteral administration.

The term parenteral as used herein includes subcutaneous, intradermal, intravascular (e.g., intravenous), intramuscular, spinal, intracranial, intrathecal, intraocular, periocular, intraorbital, intrasynovial and intraperitoneal injection, as well as any similar injection or infusion technique. The composition can be also for example a suspension, emulsion, sustained release formulation, cream, gel or powder. The composition can be formulated as a suppository, with traditional binders and carriers such as triglycerides.

Having now described the present invention in detail, the same will be more clearly understood by reference to the following examples, which are included herewith for purposes of illustration only and are not intended to be limiting of the invention. All patents and publications referred to herein are expressly incorporated by reference.

### EXAMPLES

Throughout the examples, the following materials and methods were used unless otherwise stated.

### Materials and Methods

In general, the practice of the present invention employs, unless otherwise indicated, conventional techniques of chemistry, biophysics, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., CS.H.L. Press, Pub. (1999); and Current Protocols in Molecular Biology, eds. Ausubel et al., John Wiley & Sons (1992).

### Example 1

### Evaluation of the Thrombin-Mediated D′D3 Release of Various VWF Constructs

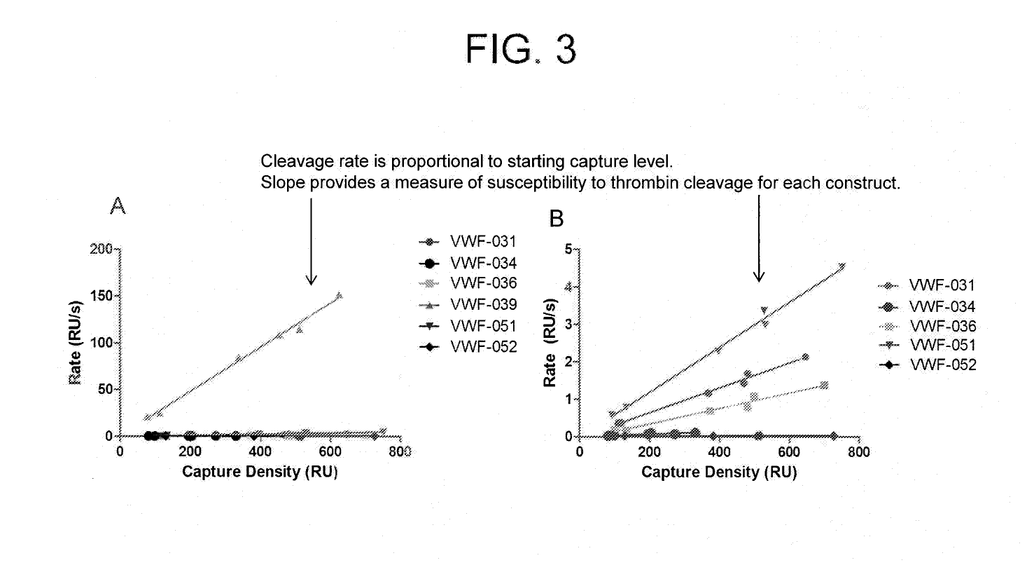

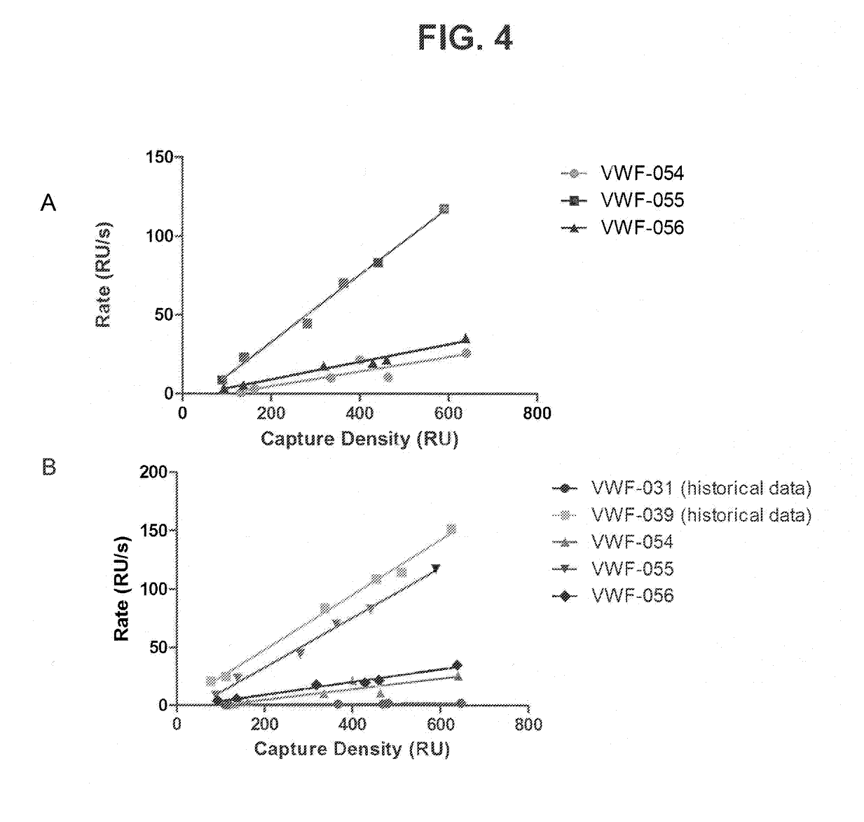

This example evaluates the kinetics of thrombin-mediated D′D3 release at 37° C. of various VWF constructs mentioned in FIG. 2. Biocore experiments were conducted with VWF-Fc constructs which contain different thrombin cleavable linker between D′D3 domain of VWF and Fc. The ultimate goal is to apply the information gathered from VWF-Fc thrombin digestion to FVIII-VWF heterodimers as described herein. All VWF-D′D3 constructs were ran over the chip to achieve the capture densities of protein ranging from 100-700 RU. After VWF construct was captured on the chip, 5 U/ml of thrombin was injected over the surface for 5 minutes. The Fc remains bound to the chip, while the D′D3 in the cleavable constructs is released. Rate (RU/s) vs. capture density (RU) was plotted as shown in FIGS. 3 and 4. Cleavage rate is proportional to starting capture density while slope provided a measure of susceptibility to thrombin cleavage for each construct.

FIG. 3 shows that VWF-052 (which does not have thrombin cleavage site in the linker region) as expected is not cleaved by thrombin. The rate of VWF-039 (LVPR with PAR1 site) is comparable to FVIII cleavage rate (data not shown). Thus, VWF-039 served as the bench mark for full D′D3 release from Fc. The ratio of slopes of various VWF-Fc constructs with respect to VWF-039 was used to determine the efficiency of thrombin cleavage. VWF-039 (LVPR with PAR1 site) is cleaved with thrombin approximately 70-80-fold faster than VWF-031 (LVPR). VWF-51 (ALRPRVV) is cleaved 1.8 fold faster than VWF-031 (LVPR). VWF-034, which contains 288 XTEN along LVPR site, displayed slower cleavage compared to VWF-031.

VWF-Fc constructs were also made by introducing different acidic region (a1, a2 and a3) of FVIII protein in the linker region. VWF-055, which contains a2 region in between D′D3 and Fc region, displayed similar thrombin cleavage as VWF-039 construct. As shown in FIG. 4, VWF-054 (alregion) and VWF-056 (a3 region) showed ˜5-fold reduced thrombin cleavage.

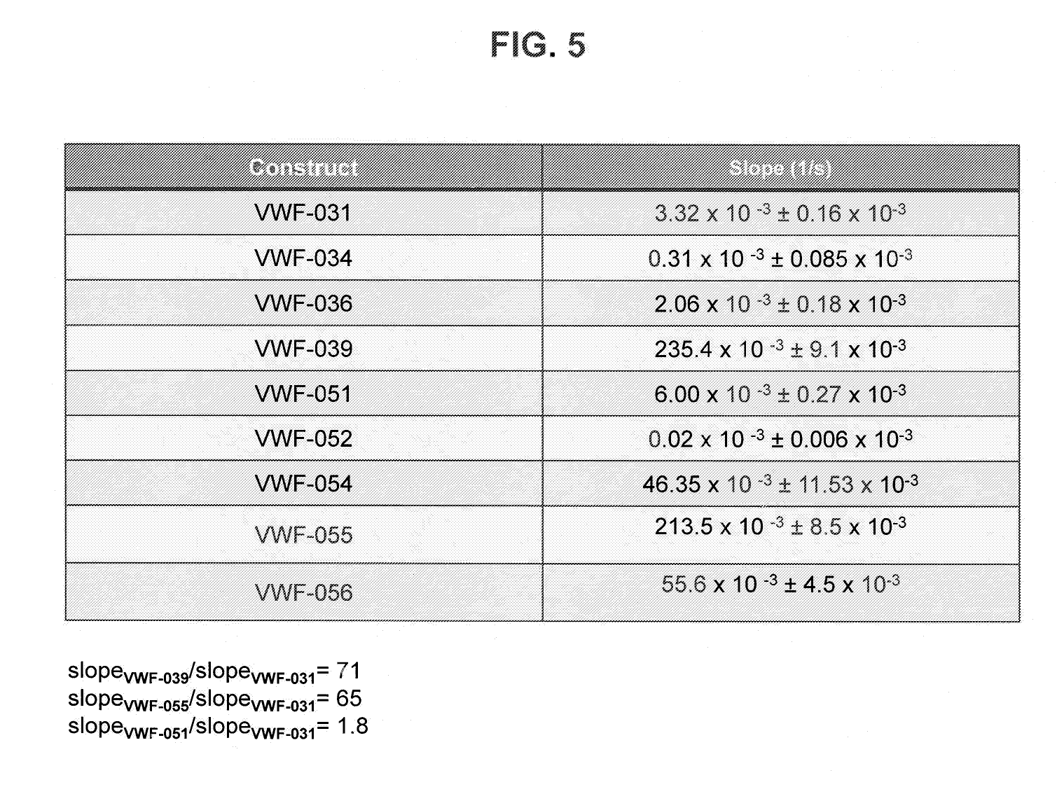

FIG. 5 shows the slope values of thrombin cleavage curves for different VWF constructs. From these results acidic region 2 (a2) of FVIII appears to be highly efficient thrombin cleavage site and was incorporated in FVIII-VWF heterodimers as described herein.

### Example 2

### Evaluation of the Hemostasis Potency of FVIII/VWFD′D3 Heterodimers with HemA Patient Whole Blood ROTEM Assay

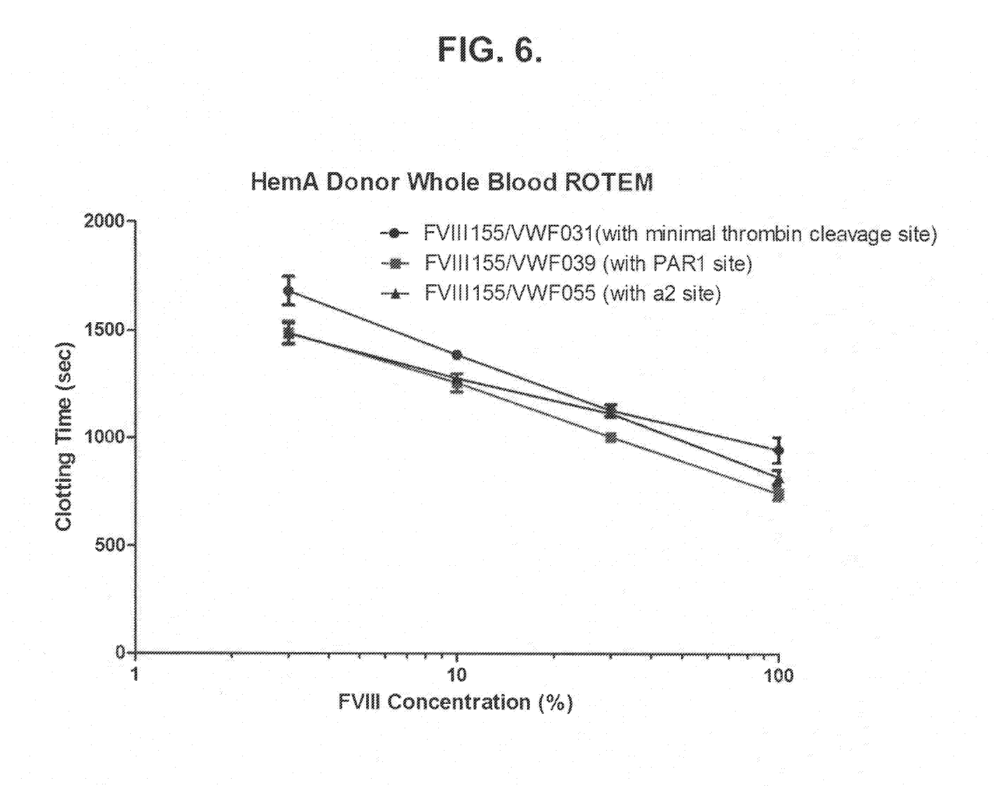

FVIII/VWFD′D3 heterodimers containing different thrombin cleavable linkers were evaluated in HemA donor whole blood ROTEM (rotational thromboelastometry) assay for their potency on hemostasis. A whole blood sample was collected from donor with severe Hemophilia A bleeding disorder with Sodium Citrate as anti-coagulant. 40 minutes after the blood sample collection, FVIII/VWFD′D3 heterodimer variants containing different thrombin cleavable linker—FVIII155/VWF031 (48aa, LVPR site), FVIII155/VWF039 (26aa, LVPR+PAR1 site), FVIII155/VWF055 (34aa, a2 from FVIII) were diluted into the whole blood sample to the final concentration at 100%, 30%, 10%, and 3% of normal as measured by FVIII chromogenic assay. Immediately after the addition of FVIII/VWFD′D3 heterodimers, ROTEM reaction was started by the addition of CaCl₂. Clotting time (time to reach 2 mm amplitude from beginning of the test) was recorded by an instrument and plotted against FVIII concentration in the samples (FIG. 6). It was hypothesized that a more potent FVIII/VWFD′D3 heterodimer will induce faster clotting process, thus resulting in a shorter clotting time compared to a less potent FVIII/VWFD′D3 heterodimer. As shown in FIG. 6, the samples with the addition of FVIII/VWF039 heterodimer had the shortest clotting time at all concentrations that had been tested, and the samples with the addition of FVIII/VWF031 heterodimer had the longest clotting time at all concentrations. The clotting time for the samples with the addition of FVIII155/VWF055 heterodimer is in the middle. Therefore, the rank of the hemostasis potency is FVIII155/VWF039>FVIII155/VWF055>FVIII155/VWF031. Since the only difference between the three molecules is the thrombin cleavable linkers between the VWF protein and the Fc region, the result indicates that the linker containing the LVPR site and the PAR1 exosite interaction motif and the a2 region of FVIII work better than the LVPR site alone.

### Example 3

### Evaluation of the Activity of FVIII/VWF Heterodimers

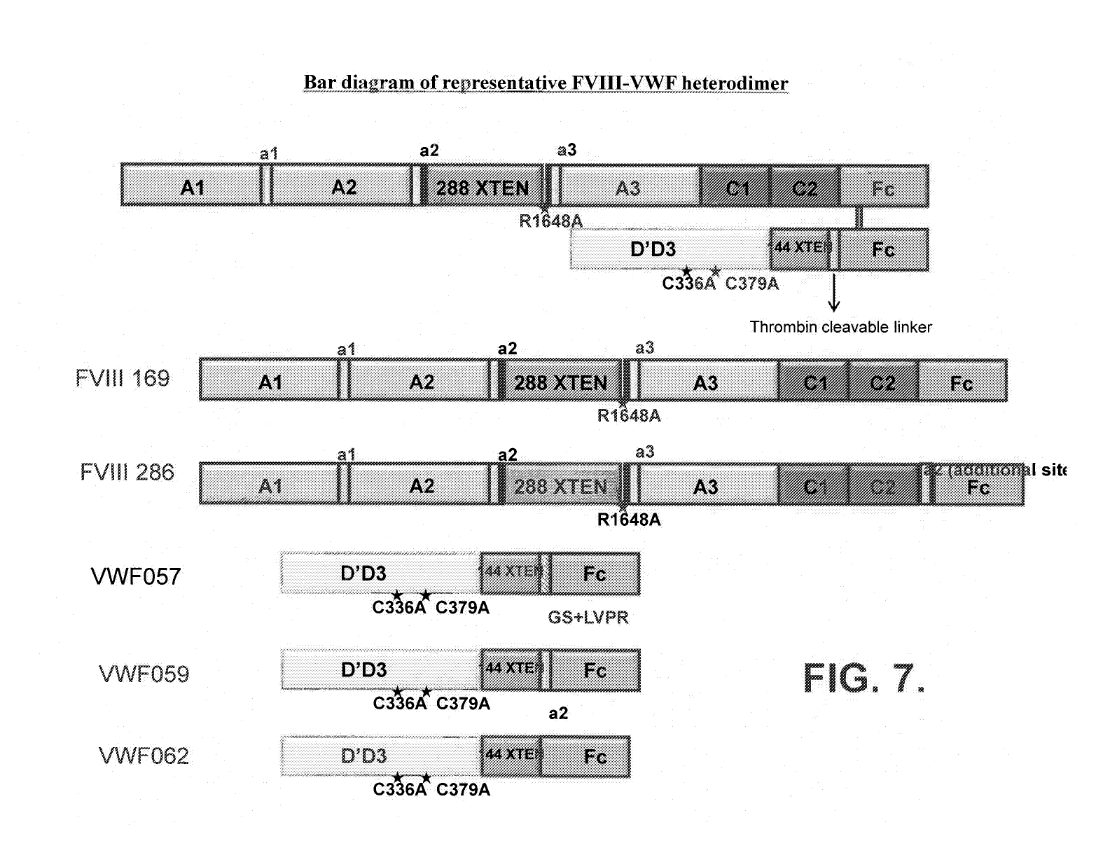

FVIII-XTEN/VWF heterodimer constructs were transfected in HEK293F cells using three plasmids: first expressing FVIII-XTEN-Fc, second expressing VWF-XTEN-Fc and third expressing PACE. Polyethylenimine (PEI) standard protocol was used for transfection and after 5 days of transfection, tissue culture media was harvested. Various combinations of FVIII-VWF heterodimers were purified form the media. Activity of the purified protein was tested in both chromogenic (two stage) and aPTT (one stage) clotting assays using standard protocols. Introduction of acidic region 2 (a2) of FVIII to either in between FVIII and Fc or between D′D3 and Fc (as shown in Table 7A and FIG. 7) improved the aPTT activity of FVIII-VWF heterodimer, as shown in Table 7C. For example, FVIII169/VWF059 heterodimer has a2 thrombin cleavage site in the D′D3-Fc linker region and has better aPTT activity than FVIII169/VWF057, which contains LVPR thrombin site in D′D3Fc linker as shown in Table 7C.

Similarly, incorporation of a2 region in between FVIII and Fc increased the one stage clotting activity of the heterodimer as evident by improved chromogenic to aPTT ratio of FVIII286/VWF059 and FVIII286/VWF062, as shown in Table 7B.

TABLE 7A

| | | | Thrombin |
|---|---|---|---|
| S. No. | Construct | | site in the linker |
| | | Linker length between | |
| | | FVIII & Fc (aa) | |
| 1 | FVIII169 | — | None |
| 2 | FVIII286 | 32 | FVIII-a2 |
| | | Linker length between | |
| | | D′D3 and Fc (aa) | |
| 1 | VWF057 | 144AE XTEN + 35 + LVPR | LVPR |
| 2 | VWF059 | 144AE XTEN + 32 | FVIII-a2 |
| 3 | VWF062 | 144AE XTEN | None |

TABLE 7B

| Constructs | Chromogenic/aPTT ratio |
|---|---|
| FVIII169/VWF057 | 2.51 |
| FVIII169/VWF059 | 1.67 |
| FVIII169/VWF062 | 2.7 |
| FVIII286/VWF059 | 0.69 |
| FVIII286/VWF062 | 0.83 |

TABLE 7C

| | Chromo specific activity | aPTT specific activity |
|---|---|---|
| Constructs | (IU/pmol) | (IU/pmol) |
| FVIII169/VWF057 | 1.60 | 0.65 |
| FVIII169/VWF059 | 1.60 | 0.90 |
| FVIII169/VWF062 | 0.87 | 0.32 |
| FVIII286/VWF059 | 1.35 | 1.96 |
| FVIII286/VWF062 | 1.08 | 1.33 |

### Example 4

### Acute Efficacy of FVIII-XTEN-Fc/D′D3-XTEN-Fc Heterodimers in HemA Mouse Tail Clip Bleeding Model

The acute efficacy of heterodimers that contain different thrombin cleavable linkers were evaluated using HemA mouse tail clip bleeding model.

8-12 weeks old male HemA mice were randomized into 5 treatment groups, and treated with a single intravenous administration of SQ BDD-FVIII, rFVIII169/VWF034, rFVIII169/VWF057, rFVIII169/VWF059 or vehicle solution, respectively. In order to mimic the episodic treatment of FVIII (to reconstitute 50-100% of normal FVIII plasma level), the selected FVIII treatment dose is 75 IU/kg as measured by FVIII aPTT activity. At this dose level, all testing FVIII variants will reconstitute ˜70% of normal murine plasma FVIII activity 5 min post dosing.

The tail clip procedure was carried out as follows. Briefly, mice were anesthetized with a 50 mg/kg Ketamine/0.5 mg/kg Dexmedetomidine cocktail prior to tail injury and placed on a 37° C. heating pad to help maintain the body temperature. The tails of the mice were then immersed in 37° C. saline for 10 minutes to dilate the lateral vein. After vein dilation, FVIII variants or vehicle solution were injected via the tail vein and the distal 5 mm of the tail was then cut off using a straight edged #11 scalpel 5 min post dosing. The shed blood was collected into 13 ml of 37° C. saline for 30 minutes and blood loss volume was determined by the weight change of the blood collection tube: blood loss volume=(collection tube end weight−beginning weight+0.10) ml. Statistical analysis were conducted using t test (Kolmogorov-Smirnov test) and one way ANOVA (KRUSKAL-Wallis test, posttest: Dunns multiple comparison test).

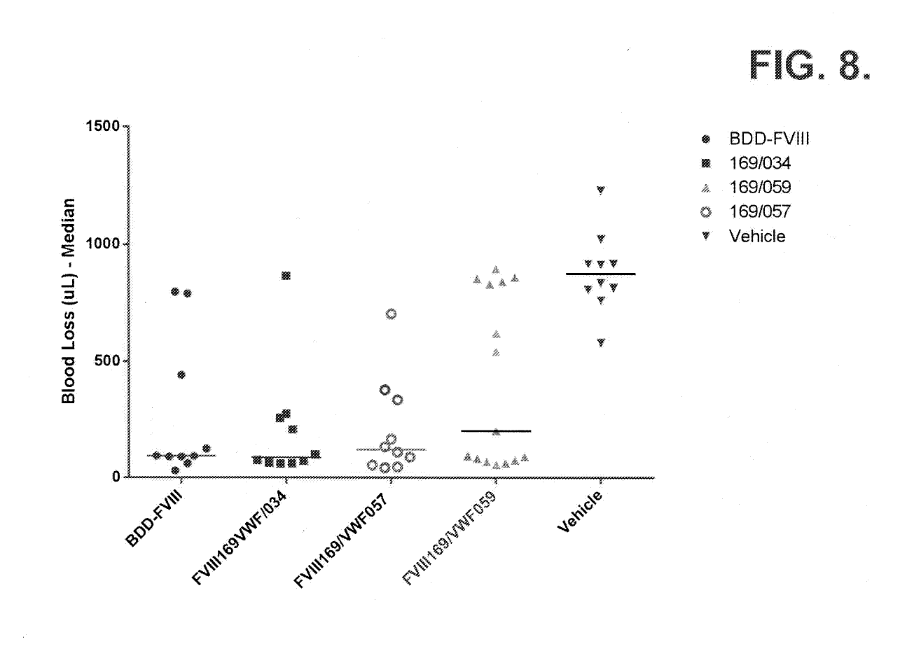

Blood loss volume from each individual animal in the study was plotted in FIG. 8. Significant reduction on blood loss volume was observed for all FVIII treatment groups compared to vehicle treated animals (p<0.05, Table 8). Similar blood loss reduction were observed from all heterodimer treatment groups compared to BDD-FVIII treatment (p>0.5, Table 8), suggesting that the heterodimer molecules could potentially be as efficacious as SQ BDD-FVIII for on demand treatment.

TABLE 8
P value of Kolmogorov-Smirnov test

| | | | FVIII169/ |
|---|---|---|---|
| | FVIII169/VWF034 | FVIII169/VWF057 | VWF059 |
| BDD-FVIII | 0.7591 | 0.9883 | 0.5176 |
| Vehicle | 0.0006 | 0.0006 | 0.0266 |

| pSYN VWF057 nucleotide sequence (VWF D′D3-Fc with LVPR | |
|---|---|
| thrombin site in the linker) | |
| (SEQ ID NO: 79) | |
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 3551 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG ATATCGGGCG CGCCAACATC AGAGAGCGCC |
| 3751 | ACCCCTGAAA GTGGTCCCGG GAGCGAGCCA GCCACATCTG GGTCGGAAAC |
| 3801 | GCCAGGCACA AGTGAGTCTG CAACTCCCGA GTCCGGACCT GGCTCCGAGC |
| 3851 | CTGCCACTAG CGGCTCCGAG ACTCCGGGAA CTTCCGAGAG CGCTACACCA |
| 3901 | GAAAGCGGAC CCGGAACCAG TACCGAACCT AGCGAGGGCT CTGCTCCGGG |
| 3951 | CAGCCCAGCC GGCTCTCCTA CATCCACGGA GGAGGGCACT TCCGAATCCG |
| 4001 | CCACCCCGGA GTCAGGGCCA GGATCTGAAC CCGCTACCTC AGGCAGTGAG |
| 4051 | ACGCCAGGAA CGAGCGAGTC CGCTACACCG GAGAGTGGGC CAGGGAGCCC |
| 4101 | TGCTGGATCT CCTACGTCCA CTGAGGAAGG GTCACCAGCG GGCTCGCCCA |
| 4151 | CCAGCACTGA AGAAGGTGCC TCGAGCGGCG GTGGAGGATC CGGTGGCGGO |
| 4201 | GGATCCGGTG GCGGGGGATC CGGTGGCGGG GGATCCGGTG GCGGGGGATC |
| 4251 | CGGTGGCGGG GGATCCCTGG TCCCCCGGGG CAGCGGAGGC GACAAAACTC |
| 4301 | ACACATGCCC ACCGTGCCCA GCTCCAGAAC TCCTGGGCGG ACCGTCAGTC |
| 4351 | TTCCTCTTCC CCCCAAAACC CAAGGACACC CTCATGATCT CCCGGACCCC |
| 4401 | TGAGGTCACA TGCGTGGTGG TGGACGTGAG CCACGAAGAC CCTGAGGTCA |
| 4451 | AGTTCAACTG GTACGTGGAC GGCGTGGAGG TGCATAATGC CAAGACAAAG |
| 4501 | CCGCGGGAGG AGCAGTACAA CAGCACGTAC CGTGTGGTCA GCGTCCTCAC |
| 4551 | CGTCCTGCAC CAGGACTGGC TGAATGGCAA GGAGTACAAG TGCAAGGTCT |
| 4601 | CCAACAAAGC CCTCCCAGCC CCCATCGAGA AAACCATCTC CAAAGCCAAA |
| 4651 | GGGCAGCCCC GAGAACCACA GGTGTACACC CTGCCCCCAT CCCGGGATGA |
| 4701 | GCTGACCAAG AACCAGGTCA GCCTGACCTG CCTGGTCAAA GGCTTCTATC |
| 4751 | CCAGCGACAT CGCCGTGGAG TGGGAGAGCA ATGGGCAGCC GGAGAACAAC |
| 4801 | TACAAGACCA CGCCTCCCGT GTTGGACTCC GACGGCTCCT TCTTCCTCTA |
| 4851 | CAGCAAGCTC ACCGTGGACA AGAGCAGGTG GCAGCAGGGG AACGTCTTCT |
| 4901 | CATGCTCCGT GATGCATGAG GCTCTGCACA ACCACTACAC GCAGAAGAGC |
| 4951 | CTCTCCCTGT CTCCGGGTAA ATGA |
| pSYN VWF057 protein sequence (VWF D′D3-Fc with LVPR | |
| thrombin site in the linker): bold underlined area | |
| shows thrombin cleavable LVPR containing linker region | |
| (SEQ ID NO: 80) | |
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP ISGAPTSESA |
| 1251 | TPESGPGSEP ATSGSETPGT SESATPESGP GSEPATSGSE TPGTSESATP |
| 1301 | ESGPGTSTEP SEGSAPGSPA GSPTSTEEGT SESATPESGP GSEPATSGSE |
| 1351 | TPGTSESATP ESGPGSPAGS PTSTEEGSPA GSPTSTEEGA SSGGGGSGGG |
| 1401 | GSGGGGSGGG GSGGGGSGGG GSGGGGSLVP RGSGGDKTHT CPPCPAPELL |
| 1451 | GGPSVFLFPP KPKDTLMISR TPEVTCVVVD VSHEDPEVKF NWYVDGVEVH |
| 1501 | NAKTKPREEQ YNSTYRVVSV LTVLHQDWLN GKEYKCKVSN KALPAPIEKT |
| 1551 | ISKAKGQPRE PQVYTLPPSR DELTKNQVSL TCLVKGFYPS DIAVEWESNG |
| 1601 | QPENNYKTTP PVLDSDGSFF LYSKLTVDKS RWQQGNVFSC SVMHEALHNH |
| 1651 | YTQKSLSLSP GK* |
| pSYN VWF059 nucleotide sequence (VWF D′D3-Fc with | |
| acidic region 2 (a2) thrombin site in the linker) | |
| (SEQ ID NO: 81) | |
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 3551 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG ATATCGGGCG CGCCAACATC AGAGAGCGCC |
| 3751 | ACCCCTGAAA GTGGTCCCGG GAGCGAGCCA GCCACATCTG GGTCGGAAAC |
| 3801 | GCCAGGCACA AGTGAGTCTG CAACTCCCGA GTCCGGACCT GGCTCCGAGC |
| 3851 | CTGCCACTAG CGGCTCCGAG ACTCCGGGAA CTTCCGAGAG CGCTACACCA |
| 3901 | GAAAGCGGAC CCGGAACCAG TACCGAACCT AGCGAGGGCT CTGCTCCGGG |
| 3951 | CAGCCCAGCC GGCTCTCCTA CATCCACGGA GGAGGGCACT TCCGAATCCG |
| 4001 | CCACCCCGGA GTCAGGGCCA GGATCTGAAC CCGCTACCTC AGGCAGTGAG |
| 4051 | ACGCCAGGAA CGAGCGAGTC CGCTACACCG GAGAGTGGGC CAGGGAGCCC |
| 4101 | TGCTGGATCT CCTACGTCCA CTGAGGAAGG GTCACCAGCG GGCTCGCCCA |
| 4151 | CCAGCACTGA AGAAGGTGCC TCGATATCTG ACAAGAACAC TGGTGATTAT |
| 4201 | TACGAGGACA GTTATGAAGA TATTTCAGCA TACTTGCTGA GTAAAAACAA |
| 4251 | TGCCATTGAA CCAAGAAGCT TCTCTGACAA AACTCACACA TGCCCACCGT |
| 4301 | GCCCAGCTCC AGAACTCCTG GGCGGACCGT CAGTCTTCCT CTTCCCCCCA |
| 4351 | AAACCCAAGG ACACCCTCAT GATCTCCCGG ACCCCTGAGG TCACATGCGT |
| 4401 | GGTGGTGGAC GTGAGCCACG AAGACCCTGA GGTCAAGTTC AACTGGTACG |
| 4451 | TGGACGGCGT GGAGGTGCAT AATGCCAAGA CAAAGCCGCG GGAGGAGCAG |
| 4501 | TACAACAGCA CGTACCGTGT GGTCAGCGTC CTCACCGTCC TGCACCAGGA |
| 4551 | CTGGCTGAAT GGCAAGGAGT ACAAGTGCAA GGTCTCCAAC AAAGCCCTCC |
| 4601 | CAGCCCCCAT CGAGAAAACC ATCTCCAAAG CCAAAGGGCA GCCCCGAGAA |
| 4651 | CCACAGGTGT ACACCCTGCC CCCATCCCGG GATGAGCTGA CCAAGAACCA |
| 4701 | GGTCAGCCTG ACCTGCCTGG TCAAAGGCTT CTATCCCAGC GACATCGCCG |
| 4751 | TGGAGTGGGA GAGCAATGGG CAGCCGGAGA ACAACTACAA GACCACGCCT |
| 4801 | CCCGTGTTGG ACTCCGACGG CTCCTTCTTC CTCTACAGCA AGCTCACCGT |
| 4851 | GGACAAGAGC AGGTGGCAGC AGGGGAACGT CTTCTCATGC TCCGTGATGC |
| 4901 | ATGAGGCTCT GCACAACCAC TACACGCAGA AGAGCCTCTC CCTGTCTCCG |
| 4951 | GGTAAATGA |
| pSYN VWF059 protein sequence (VWF D′D3-Fc with LVPR | |
| thrombin site in the linker)- bold underlined area | |
| shows a2 region | |
| (SEQ ID NO: 82) | |
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMRCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP ISGAPTSESA |
| 1251 | TPESGPGSEP ATSGSETPGT SESATPESGP GSEPATSGSE TPGTSESATP |
| 1301 | ESGPGTSTEP SEGSAPGSPA GSPTSTEEGT SESATPESGP GSEPATSGSE |
| 1351 | TPGTSESATP ESGPGSPAGS PTSTEEGSPA GSPTSTEEGA SISDKNTGDY |
| 1401 | YEDSYEDISA YLLSKNNAIE PRSFSDKTHT CPPCPAPELL GGPSVFLFPP |
| 1451 | KPKDTLMISR TPEVTCVVVD VSHEDPEVKF NWYVDGVEVH NAKTKPREEQ |
| 1501 | YNSTYRVVSV LTVLHQDWLN GKEYKCKVSN KALPAPIEKT ISKAKGQPRE |
| 1551 | PQVYTLPPSR DELTKNQVSL TCLVKGFYPS DIAVEWESNG QPENNYKTTP |
| 1601 | PVLDSDGSFF LYSKLTVDKS RWQQGNVFSC SVMHEALHNH YTQKSLSLSP |
| 1651 | GK* |
| pSYN VWF062 nucleotide sequence (VWF D′D3-Fc with no | |
| thrombin site in the linker) | |
| (SEQ ID NO: 83) | |
| 1 | ATGATTCCTG CCAGATTTGC CGGGGTGCTG CTTGCTCTGG CCCTCATTTT |
| 51 | GCCAGGGACC CTTTGTGCAG AAGGAACTCG CGGCAGGTCA TCCACGGCCC |
| 101 | GATGCAGCCT TTTCGGAAGT GACTTCGTCA ACACCTTTGA TGGGAGCATG |
| 151 | TACAGCTTTG CGGGATACTG CAGTTACCTC CTGGCAGGGG GCTGCCAGAA |
| 201 | ACGCTCCTTC TCGATTATTG GGGACTTCCA GAATGGCAAG AGAGTGAGCC |
| 251 | TCTCCGTGTA TCTTGGGGAA TTTTTTGACA TCCATTTGTT TGTCAATGGT |
| 301 | ACCGTGACAC AGGGGGACCA AAGAGTCTCC ATGCCCTATG CCTCCAAAGG |
| 351 | GCTGTATCTA GAAACTGAGG CTGGGTACTA CAAGCTGTCC GGTGAGGCCT |
| 401 | ATGGCTTTGT GGCCAGGATC GATGGCAGCG GCAACTTTCA AGTCCTGCTG |
| 451 | TCAGACAGAT ACTTCAACAA GACCTGCGGG CTGTGTGGCA ACTTTAACAT |
| 501 | CTTTGCTGAA GATGACTTTA TGACCCAAGA AGGGACCTTG ACCTCGGACC |
| 551 | CTTATGACTT TGCCAACTCA TGGGCTCTGA GCAGTGGAGA ACAGTGGTGT |
| 601 | GAACGGGCAT CTCCTCCCAG CAGCTCATGC AACATCTCCT CTGGGGAAAT |
| 651 | GCAGAAGGGC CTGTGGGAGC AGTGCCAGCT TCTGAAGAGC ACCTCGGTGT |
| 701 | TTGCCCGCTG CCACCCTCTG GTGGACCCCG AGCCTTTTGT GGCCCTGTGT |
| 751 | GAGAAGACTT TGTGTGAGTG TGCTGGGGGG CTGGAGTGCG CCTGCCCTGC |
| 801 | CCTCCTGGAG TACGCCCGGA CCTGTGCCCA GGAGGGAATG GTGCTGTACG |
| 851 | GCTGGACCGA CCACAGCGCG TGCAGCCCAG TGTGCCCTGC TGGTATGGAG |
| 901 | TATAGGCAGT GTGTGTCCCC TTGCGCCAGG ACCTGCCAGA GCCTGCACAT |
| 951 | CAATGAAATG TGTCAGGAGC GATGCGTGGA TGGCTGCAGC TGCCCTGAGG |
| 1001 | GACAGCTCCT GGATGAAGGC CTCTGCGTGG AGAGCACCGA GTGTCCCTGC |
| 1051 | GTGCATTCCG GAAAGCGCTA CCCTCCCGGC ACCTCCCTCT CTCGAGACTG |
| 1101 | CAACACCTGC ATTTGCCGAA ACAGCCAGTG GATCTGCAGC AATGAAGAAT |
| 1151 | GTCCAGGGGA GTGCCTTGTC ACTGGTCAAT CCCACTTCAA GAGCTTTGAC |
| 1201 | AACAGATACT TCACCTTCAG TGGGATCTGC CAGTACCTGC TGGCCCGGGA |
| 1251 | TTGCCAGGAC CACTCCTTCT CCATTGTCAT TGAGACTGTC CAGTGTGCTG |
| 1301 | ATGACCGCGA CGCTGTGTGC ACCCGCTCCG TCACCGTCCG GCTGCCTGGC |
| 1351 | CTGCACAACA GCCTTGTGAA ACTGAAGCAT GGGGCAGGAG TTGCCATGGA |
| 1401 | TGGCCAGGAC ATCCAGCTCC CCCTCCTGAA AGGTGACCTC CGCATCCAGC |
| 1451 | ATACAGTGAC GGCCTCCGTG CGCCTCAGCT ACGGGGAGGA CCTGCAGATG |
| 1501 | GACTGGGATG GCCGCGGGAG GCTGCTGGTG AAGCTGTCCC CCGTCTATGC |
| 1551 | CGGGAAGACC TGCGGCCTGT GTGGGAATTA CAATGGCAAC CAGGGCGACG |
| 1601 | ACTTCCTTAC CCCCTCTGGG CTGGCGGAGC CCCGGGTGGA GGACTTCGGG |
| 1651 | AACGCCTGGA AGCTGCACGG GGACTGCCAG GACCTGCAGA AGCAGCACAG |
| 1701 | CGATCCCTGC GCCCTCAACC CGCGCATGAC CAGGTTCTCC GAGGAGGCGT |
| 1751 | GCGCGGTCCT GACGTCCCCC ACATTCGAGG CCTGCCATCG TGCCGTCAGC |
| 1801 | CCGCTGCCCT ACCTGCGGAA CTGCCGCTAC GACGTGTGCT CCTGCTCGGA |
| 1851 | CGGCCGCGAG TGCCTGTGCG GCGCCCTGGC CAGCTATGCC GCGGCCTGCG |
| 1901 | CGGGGAGAGG CGTGCGCGTC GCGTGGCGCG AGCCAGGCCG CTGTGAGCTG |
| 1951 | AACTGCCCGA AAGGCCAGGT GTACCTGCAG TGCGGGACCC CCTGCAACCT |
| 2001 | GACCTGCCGC TCTCTCTCTT ACCCGGATGA GGAATGCAAT GAGGCCTGCC |
| 2051 | TGGAGGGCTG CTTCTGCCCC CCAGGGCTCT ACATGGATGA GAGGGGGGAC |
| 2101 | TGCGTGCCCA AGGCCCAGTG CCCCTGTTAC TATGACGGTG AGATCTTCCA |
| 2151 | GCCAGAAGAC ATCTTCTCAG ACCATCACAC CATGTGCTAC TGTGAGGATG |
| 2201 | GCTTCATGCA CTGTACCATG AGTGGAGTCC CCGGAAGCTT GCTGCCTGAC |
| 2251 | GCTGTCCTCA GCAGTCCCCT GTCTCATCGC AGCAAAAGGA GCCTATCCTG |
| 2301 | TCGGCCCCCC ATGGTCAAGC TGGTGTGTCC CGCTGACAAC CTGCGGGCTG |
| 2351 | AAGGGCTCGA GTGTACCAAA ACGTGCCAGA ACTATGACCT GGAGTGCATG |
| 2401 | AGCATGGGCT GTGTCTCTGG CTGCCTCTGC CCCCCGGGCA TGGTCCGGCA |
| 2451 | TGAGAACAGA TGTGTGGCCC TGGAAAGGTG TCCCTGCTTC CATCAGGGCA |
| 2501 | AGGAGTATGC CCCTGGAGAA ACAGTGAAGA TTGGCTGCAA CACTTGTGTC |
| 2551 | TGTCGGGACC GGAAGTGGAA CTGCACAGAC CATGTGTGTG ATGCCACGTG |
| 2601 | CTCCACGATC GGCATGGCCC ACTACCTCAC CTTCGACGGG CTCAAATACC |
| 2651 | TGTTCCCCGG GGAGTGCCAG TACGTTCTGG TGCAGGATTA CTGCGGCAGT |
| 2701 | AACCCTGGGA CCTTTCGGAT CCTAGTGGGG AATAAGGGAT GCAGCCACCC |
| 2751 | CTCAGTGAAA TGCAAGAAAC GGGTCACCAT CCTGGTGGAG GGAGGAGAGA |
| 2801 | TTGAGCTGTT TGACGGGGAG GTGAATGTGA AGAGGCCCAT GAAGGATGAG |
| 2851 | ACTCACTTTG AGGTGGTGGA GTCTGGCCGG TACATCATTC TGCTGCTGGG |
| 2901 | CAAAGCCCTC TCCGTGGTCT GGGACCGCCA CCTGAGCATC TCCGTGGTCC |
| 2951 | TGAAGCAGAC ATACCAGGAG AAAGTGTGTG GCCTGTGTGG GAATTTTGAT |
| 3001 | GGCATCCAGA ACAATGACCT CACCAGCAGC AACCTCCAAG TGGAGGAAGA |
| 3051 | CCCTGTGGAC TTTGGGAACT CCTGGAAAGT GAGCTCGCAG TGTGCTGACA |
| 3101 | CCAGAAAAGT GCCTCTGGAC TCATCCCCTG CCACCTGCCA TAACAACATC |
| 3151 | ATGAAGCAGA CGATGGTGGA TTCCTCCTGT AGAATCCTTA CCAGTGACGT |
| 3201 | CTTCCAGGAC TGCAACAAGC TGGTGGACCC CGAGCCATAT CTGGATGTCT |
| 3251 | GCATTTACGA CACCTGCTCC TGTGAGTCCA TTGGGGACTG CGCCGCATTC |
| 3301 | TGCGACACCA TTGCTGCCTA TGCCCACGTG TGTGCCCAGC ATGGCAAGGT |
| 3351 | GGTGACCTGG AGGACGGCCA CATTGTGCCC CCAGAGCTGC GAGGAGAGGA |
| 3401 | ATCTCCGGGA GAACGGGTAT GAGGCTGAGT GGCGCTATAA CAGCTGTGCA |
| 3451 | CCTGCCTGTC AAGTCACGTG TCAGCACCCT GAGCCACTGG CCTGCCCTGT |
| 3501 | GCAGTGTGTG GAGGGCTGCC ATGCCCACTG CCCTCCAGGG AAAATCCTGG |
| 3551 | ATGAGCTTTT GCAGACCTGC GTTGACCCTG AAGACTGTCC AGTGTGTGAG |
| 3601 | GTGGCTGGCC GGCGTTTTGC CTCAGGAAAG AAAGTCACCT TGAATCCCAG |
| 3651 | TGACCCTGAG CACTGCCAGA TTTGCCACTG TGATGTTGTC AACCTCACCT |
| 3701 | GTGAAGCCTG CCAGGAGCCG ATATCGGGCG CGCCAACATC AGAGAGCGCC |
| 3751 | ACCCCTGAAA GTGGTCCCGG GAGCGAGCCA GCCACATCTG GGTCGGAAAC |
| 3801 | GCCAGGCACA AGTGAGTCTG CAACTCCCGA GTCCGGACCT GGCTCCGAGC |
| 3851 | CTGCCACTAG CGGCTCCGAG ACTCCGGGAA CTTCCGAGAG CGCTACACCA |
| 3901 | GAAAGCGGAC CCGGAACCAG TACCGAACCT AGCGAGGGCT CTGCTCCGGG |
| 3951 | CAGCCCAGCC GGCTCTCCTA CATCCACGGA GGAGGGCACT TCCGAATCCG |
| 4001 | CCACCCCGGA GTCAGGGCCA GGATCTGAAC CCGCTACCTC AGGCAGTGAG |
| 4051 | ACGCCAGGAA CGAGCGAGTC CGCTACACCG GAGAGTGGGC CAGGGAGCCC |
| 4101 | TGCTGGATCT CCTACGTCCA CTGAGGAAGG GTCACCAGCG GGCTCGCCCA |
| 4151 | CCAGCACTGA AGAAGGTGCC TCGAGCGACA AAACTCACAC ATGCCCACCG |
| 4201 | TGCCCAGCTC CAGAACTCCT GGGCGGACCG TCAGTCTTCC TCTTCCCCCC |
| 4251 | AAAACCCAAG GACACCCTCA TGATCTCCCG GACCCCTGAG GTCACATGCG |
| 4301 | TGGTGGTGGA CGTGAGCCAC GAAGACCCTG AGGTCAAGTT CAACTGGTAC |
| 4351 | GTGGACGGCG TGGAGGTGCA TAATGCCAAG ACAAAGCCGC GGGAGGAGCA |
| 4401 | GTACAACAGC ACGTACCGTG TGGTCAGCGT CCTCACCGTC CTGCACCAGG |
| 4451 | ACTGGCTGAA TGGCAAGGAG TACAAGTGCA AGGTCTCCAA CAAAGCCCTC |
| 4501 | CCAGCCCCCA TCGAGAAAAC CATCTCCAAA GCCAAAGGGC AGCCCCGAGA |
| 4551 | ACCACAGGTG TACACCCTGC CCCCATCCCG GGATGAGCTG ACCAAGAACC |
| 4601 | AGGTCAGCCT GACCTGCCTG GTCAAAGGCT TCTATCCCAG CGACATCGCC |
| 4651 | GTGGAGTGGG AGAGCAATGG GCAGCCGGAG AACAACTACA AGACCACGCC |
| 4701 | TCCCGTGTTG GACTCCGACG GCTCCTTCTT CCTCTACAGC AAGCTCACCG |
| 4751 | TGGACAAGAG CAGGTGGCAG CAGGGGAACG TCTTCTCATG CTCCGTGATG |
| 4801 | CATGAGGCTC TGCACAACCA CTACACGCAG AAGAGCCTCT CCCTGTCTCC |
| 4851 | GGGTAAATGA |
| pSYN VWF062 protein sequence (VWF D′D3-Fc with no | |
| thrombin site in the linker) | |
| (SEQ ID NO: 84) | |
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVRLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGPMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP ISGAPTSESA |
| 1251 | TPESGPGSEP ATSGSETPGT SESATPESGP GSEPATSGSE TPGTSESATP |
| 1301 | ESGPGTSTEP SEGSAPGSPA GSPTSTEEGT SESATPESGP GSEPATSGSE |
| 1351 | TPGTSESATP ESGPGSPAGS PTSTEEGSPA GSPTSTEEGA SSDKTHTCPP |
| 1401 | CPAPELLGGP SVFLFPPKPK DTLMISRTPE VTCVVVDVSH EDPEVKFNWY |
| 1451 | VDGVEVHNAK TKPREEQYNS TYRVVSVLTV LHQDWLNGKE YKCKVSNKAL |
| 1501 | PAPIEKTISK AKGQPREPQV YTLPPSRDEL TKNQVSLTCL VKGFYPSDIA |
| 1551 | VEWESNGQPE NNYKTTPPVL DSDGSFFLYS KLTVDKSRWQ QGNVFSCSVM |
| 1601 | HEALHNHYTQ KSLSLSPGK* |
| pSYN FVIII 286 nucleotide sequence (FVIII-Fc with | |
| additional a2 region in between FVIII and Fc) | |
| (SEQ ID NO: 85) | |
| 1 | ATGCAAATAG AGCTCTCCAC CTGCTTCTTT CTGTGCCTTT TGCGATTCTG |
| 51 | CTTTAGTGCC ACCAGAAGAT ACTACCTGGG TGCAGTGGAA CTGTCATGGG |
| 101 | ACTATATGCA AAGTGATCTC GGTGAGCTGC CTGTGGACGC AAGATTTCCT |
| 151 | CCTAGAGTGC CAAAATCTTT TCCATTCAAC ACCTCAGTCG TGTACAAAAA |
| 201 | GACTCTGTTT GTAGAATTCA CGGATCACCT TTTCAACATC GCTAAGCCAA |
| 251 | GGCCACCCTG GATGGGTCTG CTAGGTCCTA CCATCCAGGC TGAGGTTTAT |
| 301 | GATACAGTGG TCATTACACT TAAGAACATG GCTTCCCATC CTGTCAGTCT |
| 351 | TCATGCTGTT GGTGTATCCT ACTGGAAAGC TTCTGAGGGA GCTGAATATG |
| 401 | ATGATCAGAC CAGTCAAAGG GAGAAAGAAG ATGATAAAGT CTTCCCTGGT |
| 451 | GGAAGCCATA CATATGTCTG GCAGGTCCTG AAAGAGAATG GTCCAATGGC |
| 501 | CTCTGACCCA CTGTGCCTTA CCTACTCATA TCTTTCTCAT GTGGACCTGG |
| 551 | TAAAAGACTT GAATTCAGGC CTCATTGGAG CCCTACTAGT ATGTAGAGAA |
| 601 | GGGAGTCTGG CCAAGGAAAA GACACAGACC TTGCACAAAT TTATACTACT |
| 651 | TTTTGCTGTA TTTGATGAAG GGAAAAGTTG GCACTCAGAA ACAAAGAACT |
| 701 | CCTTGATGCA GGATAGGGAT GCTGCATCTG CTCGGGCCTG GCCTAAAATG |
| 751 | CACACAGTCA ATGGTTATGT AAACAGGTCT CTGCCAGGTC TGATTGGATG |
| 801 | CCACAGGAAA TCAGTCTATT GGCATGTGAT TGGAATGGGC ACCACTCCTG |
| 851 | AAGTGCACTC AATATTCCTC GAAGGTCACA CATTTCTTGT GAGGAACCAT |
| 901 | CGCCAGGCTA GCTTGGAAAT CTCGCCAATA ACTTTCCTTA CTGCTCAAAC |
| 951 | ACTCTTGATG GACCTTGGAC AGTTTCTACT GTTTTGTCAT ATCTCTTCCC |
| 1001 | ACCAACATGA TGGCATGGAA GCTTATGTCA AAGTAGACAG CTGTCCAGAG |
| 1051 | GAACCCCAAC TACGAATGAA AAATAATGAA GAAGCGGAAG ACTATGATGA |
| 1101 | TGATCTTACT GATTCTGAAA TGGATGTGGT CAGGTTTGAT GATGACAACT |
| 1151 | CTCCTTCCTT TATCCAAATT CGCTCAGTTG CCAAGAAGCA TCCTAAAACT |
| 1201 | TGGGTACATT ACATTGCTGC TGAAGAGGAG GACTGGGACT ATGCTCCCTT |
| 1251 | AGTCCTCGCC CCCGATGACA GAAGTTATAA AAGTCAATAT TTGAACAATG |
| 1301 | GCCCTCAGCG GATTGGTAGG AAGTACAAAA AAGTCCGATT TATGGCATAC |
| 1351 | ACAGATGAAA CCTTTAAGAC TCGTGAAGCT ATTCAGCATG AATCAGGAAT |
| 1401 | CTTGGGACCT TTACTTTATG GGGAAGTTGG AGACACACTG TTGATTATAT |
| 1451 | TTAAGAATCA AGCAAGCAGA CCATATAACA TCTACCCTCA CGGAATCACT |
| 1501 | GATGTCCGTC CTTTGTATTC AAGGAGATTA CCAAAAGGTG TAAAACATTT |
| 1551 | GAAGGATTTT CCAATTCTGC CAGGAGAAAT ATTCAAATAT AAATGGACAG |
| 1601 | TGACTGTAGA AGATGGGCCA ACTAAATCAG ATCCTCGGTG CCTGACCCGC |
| 1651 | TATTACTCTA GTTTCGTTAA TATGGAGAGA GATCTAGCTT CAGGACTCAT |
| 1701 | TGGCCCTCTC CTCATCTGCT ACAAAGAATC TGTAGATCAA AGAGGAAACC |
| 1751 | AGATAATGTC AGACAAGAGG AATGTCATCC TGTTTTCTGT ATTTGATGAG |
| 1801 | AACCGAAGCT GGTACCTCAC AGAGAATATA CAACGCTTTC TCCCCAATCC |
| 1851 | AGCTGGAGTG CAGCTTGAGG ATCCAGAGTT CCAAGCCTCC AACATCATGC |
| 1901 | ACAGCATCAA TGGCTATGTT TTTGATAGTT TGCAGTTGTC AGTTTGTTTG |
| 1951 | CATGAGGTGG CATACTGGTA CATTCTAAGC ATTGGAGCAC AGACTGACTT |
| 2001 | CCTTTCTGTC TTCTTCTCTG GATATACCTT CAAACACAAA ATGGTCTATG |
| 2051 | AAGACACACT CACCCTATTC CCATTCTCAG GAGAAACTGT CTTCATGTCG |
| 2101 | ATGGAAAACC CAGGTCTATG GATTCTGGGG TGCCACAACT CAGACTTTCG |
| 2151 | GAACAGAGGC ATGACCGCCT TACTGAAGGT TTCTAGTTGT GACAAGAACA |
| 2201 | CTGGTGATTA TTACGAGGAC AGTTATGAAG ATATTTCAGC ATACTTGCTG |
| 2251 | AGTAAAAACA ATGCCATTGA ACCAAGAAGC TTCTCTCAAA ACGGCGCGCC |
| 2301 | AGGTACCTCA GAGTCTGCTA CCCCCGAGTC AGGGCCAGGA TCAGAGCCAG |
| 2351 | CCACCTCCGG GTCTGAGACA CCCGGGACTT CCGAGAGTGC CACCCCTGAG |
| 2401 | TCCGGACCCG GGTCCGAGCC CGCCACTTCC GGCTCCGAAA CTCCCGGCAC |
| 2451 | AAGCGAGAGC GCTACCCCAG AGTCAGGACC AGGAACATCT ACAGAGCCCT |
| 2501 | CTGAAGGCTC CGCTCCAGGG TCCCCAGCCG GCAGTCCCAC TAGCACCGAG |
| 2551 | GAGGGAACCT CTGAAAGCGC CACACCCGAA TCAGGGCCAG GGTCTGAGCC |
| 2601 | TGCTACCAGC GGCAGCGAGA CACCAGGCAC CTCTGAGTCC GCCACACCAG |
| 2651 | AGTCCGGACC CGGATCTCCC GCTGGGAGCC CCACCTCCAC TGAGGAGGGA |
| 2701 | TCTCCTGCTG GCTCTCCAAC ATCTACTGAG GAAGGTACCT CAACCGAGCC |
| 2751 | ATCCGAGGGA TCAGCTCCCG GCACCTCAGA GTCGGCAACC CCGGAGTCTG |
| 2801 | GACCCGGAAC TTCCGAAAGT GCCACACCAG AGTCCGGTCC CGGGACTTCA |
| 2851 | GAATCAGCAA CACCCGAGTC CGGCCCTGGG TCTGAACCCG CCACAAGTGG |
| 2901 | TAGTGAGACA CCAGGATCAG AACCTGCTAC CTCAGGGTCA GAGACACCCG |
| 2951 | GATCTCCGGC AGGCTCACCA ACCTCCACTG AGGAGGGCAC CAGCACAGAA |
| 3001 | CCAAGCGAGG GCTCCGCACC CGGAACAAGC ACTGAACCCA GTGAGGGTTC |
| 3051 | AGCACCCGGC TCTGAGCCGG CCACAAGTGG CAGTGAGACA CCCGGCACTT |
| 3101 | CAGAGAGTGC CACCCCCGAG AGTGGCCCAG GCACTAGTAC CGAGCCCTCT |
| 3151 | GAAGGCAGTG CGCCAGCCTC GAGCCCACCA GTCTTGAAAC GCCATCAAGC |
| 3201 | TGAAATAACT CGTACTACTC TTCAGTCAGA TCAAGAGGAA ATCGATTATG |
| 3251 | ATGATACCAT ATCAGTTGAA ATGAAGAAGG AAGATTTTGA CATTTATGAT |
| 3301 | GAGGATGAAA ATCAGAGCCC CCGCAGCTTT CAAAAGAAAA CACGACACTA |
| 3351 | TTTTATTGCT GCAGTGGAGA GGCTCTGGGA TTATGGGATG AGTAGCTCCC |
| 3401 | CACATGTTCT AAGAAACAGG GCTCAGAGTG GCAGTGTCCC TCAGTTCAAG |
| 3451 | AAAGTTGTTT TCCAGGAATT TACTGATGGC TCCTTTACTC AGCCCTTATA |
| 3501 | CCGTGGAGAA CTAAATGAAC ATTTGGGACT CCTGGGGCCA TATATAAGAG |
| 3551 | CAGAAGTTGA AGATAATATC ATGGTAACTT TCAGAAATCA GGCCTCTCGT |
| 3601 | CCCTATTCCT TCTATTCTAG CCTTATTTCT TATGAGGAAG ATCAGAGGCA |
| 3651 | AGGAGCAGAA CCTAGAAAAA ACTTTGTCAA GCCTAATGAA ACCAAAACTT |
| 3701 | ACTTTTGGAA AGTGCAACAT CATATGGCAC CCACTAAAGA TGAGTTTGAC |
| 3751 | TGCAAAGCCT GGGCTTATTT CTCTGATGTT GACCTGGAAA AAGATGTGCA |
| 3801 | CTCAGGCCTG ATTGGACCCC TTCTGGTCTG CCACACTAAC ACACTGAACC |
| 3851 | CTGCTCATGG GAGACAAGTG ACAGTACAGG AATTTGCTCT GTTTTTCACC |
| 3901 | ATCTTTGATG AGACCAAAAG CTGGTACTTC ACTGAAAATA TGGAAAGAAA |
| 3951 | CTGCAGGGCT CCCTGCAATA TCCAGATGGA AGATCCCACT TTTAAAGAGA |
| 4001 | ATTATCGCTT CCATGCAATC AATGGCTACA TAATGGATAC ACTACCTGGC |
| 4051 | TTAGTAATGG CTCAGGATCA AAGGATTCGA TGGTATCTGC TCAGCATGGG |
| 4101 | CAGCAATGAA AACATCCATT CTATTCATTT CAGTGGACAT GTGTTCACTG |
| 4151 | TACGAAAAAA AGAGGAGTAT AAAATGGCAC TGTACAATCT CTATCCAGGT |
| 4201 | GTTTTTGAGA CAGTGGAAAT GTTACCATCC AAAGCTGGAA TTTGGCGGGT |
| 4251 | GGAATGCCTT ATTGGCGAGC ATCTACATGC TGGGATGAGC ACACTTTTTC |
| 4301 | TGGTGTACAG CAATAAGTGT CAGACTCCCC TGGGAATGGC TTCTGGACAC |
| 4351 | ATTAGAGATT TTCAGATTAC AGCTTCAGGA CAATATGGAC AGTGGGCCCC |
| 4401 | AAAGCTGGCC AGACTTCATT ATTCCGGATC AATCAATGCC TGGAGCACCA |
| 4451 | AGGAGCCCTT TTCTTGGATC AAGGTGGATC TGTTGGCACC AATGATTATT |
| 4501 | CACGGCATCA AGACCCAGGG TGCCCGTCAG AAGTTCTCCA GCCTCTACAT |
| 4551 | CTCTCAGTTT ATCATCATGT ATAGTCTTGA TGGGAAGAAG TGGCAGACTT |
| 4601 | ATCGAGGAAA TTCCACTGGA ACCTTAATGG TCTTCTTTGG CAATGTGGAT |
| 4651 | TCATCTGGGA TAAAACACAA TATTTTTAAC CCTCCAATTA TTGCTCGATA |
| 4701 | CATCCGTTTG CACCCAACTC ATTATAGCAT TCGCAGCACT CTTCGCATGG |
| 4751 | AGTTGATGGG CTGTGATTTA AATAGTTGCA GCATGCCATT GGGAATGGAG |
| 4801 | AGTAAAGCAA TATCAGATGC ACAGATTACT GCTTCATCCT ACTTTACCAA |
| 4851 | TATGTTTGCC ACCTGGTCTC CTTCAAAAGC TCGACTTCAC CTCCAAGGGA |
| 4901 | GGAGTAATGC CTGGAGACCT CAGGTGAATA ATCCAAAAGA GTGGCTGCAA |
| 4951 | GTGGACTTCC AGAAGACAAT GAAAGTCACA GGAGTAACTA CTCAGGGAGT |
| 5001 | AAAATCTCTG CTTACCAGCA TGTATGTGAA GGAGTTCCTC ATCTCCAGCA |
| 5051 | GTCAAGATGG CCATCAGTGG ACTCTCTTTT TTCAGAATGG CAAAGTAAAG |
| 5101 | GTTTTTCAGG GAAATCAAGA CTCCTTCACA CCTGTGGTGA ACTCTCTAGA |
| 5151 | CCCACCGTTA CTGACTCGCT ACCTTCGAAT TCACCCCCAG AGTTGGGTGC |
| 5201 | ACCAGATTGC CCTGAGGATG GAGGTTCTGG GCTGCGAGGC ACAGGACCTC |
| 5251 | TACGACAAGA ACACTGGTGA TTATTACGAG GACAGTTATG AAGATATTTC |
| 5301 | AGCATACTTG CTGAGTAAAA ACAATGCCAT TGAACCAAGA AGCTTCTCTG |
| 5351 | ACAAAACTCA CACATGCCCA CCGTGCCCAG CTCCAGAACT CCTGGGCGGA |
| 5401 | CCGTCAGTCT TCCTCTTCCC CCCAAAACCC AAGGACACCC TCATGATCTC |
| 5451 | CCGGACCCCT GAGGTCACAT GCGTGGTGGT GGACGTGAGC CACGAAGACC |
| 5501 | CTGAGGTCAA GTTCAACTGG TACGTGGACG GCGTGGAGGT GCATAATGCC |
| 5551 | AAGACAAAGC CGCGGGAGGA GCAGTACAAC AGCACGTACC GTGTGGTCAG |
| 5601 | CGTCCTCACC GTCCTGCACC AGGACTGGCT GAATGGCAAG GAGTACAAGT |
| 5651 | GCAAGGTCTC CAACAAAGCC CTCCCAGCCC CCATCGAGAA AACCATCTCC |
| 5701 | AAAGCCAAAG GGCAGCCCCG AGAACCACAG GTGTACACCC TGCCCCCATC |
| 5751 | CCGGGATGAG CTGACCAAGA ACCAGGTCAG CCTGACCTGC CTGGTCAAAG |
| 5801 | GCTTCTATCC CAGCGACATC GCCGTGGAGT GGGAGAGCAA TGGGCAGCCG |
| 5851 | GAGAACAACT ACAAGACCAC GCCTCCCGTG TTGGACTCCG ACGGCTCCTT |
| 5901 | CTTCCTCTAC AGCAAGCTCA CCGTGGACAA GAGCAGGTGG CAGCAGGGGA |
| 5951 | ACGTCTTCTC ATGCTCCGTG ATGCATGAGG CTCTGCACAA CCACTACACG |
| 6001 | CAGAAGAGCC TCTCCCTGTC TCCGGGTAAA TGA |
| pSYN FVIII 286 protein sequence (FVIII-Fc with | |
| additional a2 region in between FVIII and Fc; | |
| shown in bold and underline) | |
| (SEQ ID NO: 86) | |
| 1 | ATRRYYLGAV ELSWDYMQSD LGELPVDARF PPRVPKSFPF NTSVVYKKTL |
| 51 | FVEFTDHLFN IAKPRPPWMG LLGPTIQAEV YDTVVITLKN MASHPVSLHA |
| 101 | VGVSYWKASE GAEYDDQTSQ REKEDDKVFP GGSHTYVWQV LKENGPMASD |
| 151 | PLCLTYSYLS HVDLVKDLNS GLIGALLVCR EGSLAKEKTQ TLHKFILLFA |
| 201 | VFDEGKSWHS ETKNSLMQDR DAASARAWPK MHTVNGYVNR SLPGLIGCHR |
| 251 | KSVYWHVIGM GTTPEVHSIF LEGHTFLVRN HRQASLEISP ITFLTAQTLL |
| 301 | MDLGQFLLFC HISSHQHDGM EAYVKVDSCP EEPQLRMKNN EEAEDYDDDL |
| 351 | TDSEMDVVRF DDDNSPSFIQ IRSVAKKHPK TWVHYIAAEE EDWDYAPLVL |
| 401 | APDDRSYKSQ YLNNGPQRIG RKYKKVRFMA YTDETFKTRE AIQHESGILG |
| 451 | PLLYGEVGDT LLIIFKNQAS RPYNIYPHGI TDVRPLYSRR LPKGVKHLKD |
| 501 | FPILPGEIFK YKWTVTVEDG PTKSDPRCLT RYYSSFVNME RDLASGLIGP |
| 551 | LLICYKESVD QRGNQIMSDK RNVILFSVFD ENRSWYLTEN IQRFLPNPAG |
| 601 | VQLEDPEFQA SNIMHSINGY VFDSLQLSVC LHEVAYWYIL SIGAQTDFLS |
| 651 | VFFSGYTFKH KMVYEDTLTL FPFSGETVFM SMENPGLWIL GCHNSDFRNR |
| 701 | GMTALLKVSS CDKNTGDYYE DSYEDISAYL LSKNNAIEPR SFSQNGAPGT |
| 751 | SESATPESGP GSEPATSGSE TPGTSESATP ESGPGSEPAT SGSETPGTSE |
| 801 | SATPESGPGT STEPSEGSAP GSPAGSPTST EEGTSESATP ESGPGSEPAT |
| 851 | SGSETPGTSE SATPESGPGS PAGSPTSTEE GSPAGSPTST EEGTSTEPSE |
| 901 | GSAPGTSESA TPESGPGTSE SATPESGPGT SESATPESGP GSEPATSGSE |
| 951 | TPGSEPATSG SETPGSPAGS PTSTEEGTST EPSEGSAPGT STEPSEGSAP |
| 1001 | GSEPATSGSE TPGTSESATP ESGPGTSTEP SEGSAPASSP PVLKRHQAEI |
| 1051 | TRTTLQSDQE EIDYDDTISV EMKKEDFDIY DEDENQSPRS FQKKTRHYFI |
| 1101 | AAVERLWDYG MSSSPHVLRN RAQSGSVPQF KKVVFQEFTD GSFTQPLYRG |
| 1151 | ELNEHLGLLG PYIRAEVEDN IMVTFRNQAS RPYSFYSSLI SYEEDQRQGA |
| 1201 | EPRKNFVKPN ETKTYFWKVQ HHMAPTKDEF DCKAWAYFSD VDLEKDVHSG |
| 1251 | LIGPLLVCHT NTLNPAHGRQ VTVQEFALFF TIFDETKSWY FTENMERNCR |
| 1301 | APCNIQMEDP TFKENYRFHA INGYIMDTLP GLVMAQDQRI RWYLLSMGSN |
| 1351 | ENIHSIHFSG HVFTVRKKEE YKMALYNLYP GVFETVEMLP SKAGIWRVEC |
| 1401 | LIGEHLHAGM STLFLVYSNK CQTPLGMASG HIRDFQITAS GQYGQWAPKL |
| 1451 | ARLHYSGSIN AWSTKEPFSW IKVDLLAPMI IHGIKTQGAR QKFSSLYISQ |
| 1501 | FIIMYSLDGK KWQTYRGNST GTLMVFFGNV DSSGIKHNIF NPPIIARYIR |
| 1551 | LHPTHYSIRS TLRMELMGCD LNSCSMPLGM ESKAISDAQI TASSYFTNMF |
| 1601 | ATWSPSKARL HLQGRSNAWR PQVNNPKEWL QVDFQKTMKV TGVTTQGVKS |
| 1651 | LLTSMYVKEF LISSSQDGHQ WTLFFQNGKV KVFQGNQDSF TPVVNSLDPP |
| 1701 | LLTRYLRIHP QSWVHQIALR MEVLGCEAQD LYDKNTGDYY EDSYEDISAY |
| 1751 | LLSKNNAIEP RSFSDKTHTC PPCPAPELLG GPSVFLFPPK PKDTLMISRT |
| 1801 | PEVTCVVVDV SHEDPEVKFN WYVDGVEVHN AKTKPREEQY NSTYRVVSVL |
| 1851 | TVLHQDWLNG KEYKCKVSNK ALPAPIEKTI SKAKGQPREP QVYTLPPSRD |
| 1901 | ELTKNQVSLT CLVKGFYPSD IAVEWESNGQ PENNYKTTPP VLDSDGSFFL |
| 1951 | YSKLTVDKSR WQQGNVFSCS VMHEALHNHY TQKSLSLSPG K* |
| FVIII 169 nucleotide sequence | |
| (SEQ ID NO: 87) | |
| 1 | ATGCA AATAG AGCTC TCCAC CTGCT TCTTT CTGTG CCTTT TGCGA TTCTG |
| 51 | CTTTA GTGCC ACCAG AAGAT ACTAC CTGGG TGCAG TGGAA CTGTC ATGGG |
| 101 | ACTAT ATGCA AAGTG ATCTC GGTGA GCTGC CTGTG GACGC AAGAT TTCCT |
| 151 | CCTAG AGTGC CAAAA TCTTT TCCAT TCAAC ACCTC AGTCG TGTAC AAAAA |
| 201 | GACTC TGTTT GTAGA ATTCA CGGAT CACCT TTTCA ACATC GCTAA GCCAA |
| 251 | GGCCA CCCTG GATGG GTCTG CTAGG TCCTA CCATC CAGGC TGAGG TTTAT |
| 301 | GATAC AGTGG TCATT ACACT TAAGA ACATG GCTTC CCATC CTGTC AGTCT |
| 351 | TCATG CTGTT GGTGT ATCCT ACTGG AAAGC TTCTG AGGGA GCTGA ATATG |
| 401 | ATGAT CAGAC CAGTC AAAGG GAGAA AGAAG ATGAT AAAGT CTTCC CTGGT |
| 451 | GGAAG CCATA CATAT GTCTG GCAGG TCCTG AAAGA GAATG GTCCA ATGGC |
| 501 | CTCTG ACCCA CTGTG CCTTA CCTAC TCATA TCTTT CTCAT GTGGA CCTGG |
| 551 | TAAAA GACTT GAATT CAGGC CTCAT TGGAG CCCTA CTAGT ATGTA GAGAA |
| 601 | GGGAG TCTGG CCAAG GAAAA GACAC AGACC TTGCA CAAAT TTATA CTACT |
| 651 | TTTTG CTGTA TTTGA TGAAG GGAAA AGTTG GCACT CAGAA ACAAA GAACT |
| 701 | CCTTG ATGCA GGATA GGGAT GCTGC ATCTG CTCGG GCCTG GCCTA AAATG |
| 751 | CACAC AGTCA ATGGT TATGT AAACA GGTCT CTGCC AGGTC TGATT GGATG |
| 801 | CCACA GGAAA TCAGT CTATT GGCAT GTGAT TGGAA TGGGC ACCAC TCCTG |
| 851 | AAGTG CACTC AATAT TCCTC GAAGG TCACA CATTT CTTGT GAGGA ACCAT |
| 901 | CGCCA GGCTA GCTTG GAAAT CTCGC CAATA ACTTT CCTTA CTGCT CAAAC |
| 951 | ACTCT TGATG GACCT TGGAC AGTTT CTACT GTTTT GTCAT ATCTC TTCCC |
| 1001 | ACCAA CATGA TGGCA TGGAA GCTTA TGTCA AAGTA GACAG CTGTC CAGAG |
| 1051 | GAACC CCAAC TACGA ATGAA AAATA ATGAA GAAGC GGAAG ACTAT GATGA |
| 1101 | TGATC TTACT GATTC TGAAA TGGAT GTGGT CAGGT TTGAT GATGA CAACT |
| 1151 | CTCCT TCCTT TATCC AAATT CGCTC AGTTG CCAAG AAGCA TCCTA AAACT |
| 1201 | TGGGT ACATT ACATT GCTGC TGAAG AGGAG GACTG GGACT ATGCT CCCTT |
| 1251 | AGTCC TCGCC CCCGA TGACA GAAGT TATAA AAGTC AATAT TTGAA CAATG |
| 1301 | GCCCT CAGCG GATTG GTAGG AAGTA CAAAA AAGTC CGATT TATGG CATAC |
| 1351 | ACAGA TGAAA CCTTT AAGAC TCGTG AAGCT ATTCA GCATG AATCA GGAAT |
| 1401 | CTTGG GACCT TTACT TTATG GGGAA GTTGG AGACA CACTG TTGAT TATAT |
| 1451 | TTAAG AATCA AGCAA GCAGA CCATA TAACA TCTAC CCTCA CGGAA TCACT |
| 1501 | GATGT CCGTC CTTTG TATTC AAAGA GATTA CCAAA AGGTG TAAAA CATTT |
| 1551 | GAAGG ATTTT CCAAT TCTGC CAGGA GAAAT ATTCA AATAT AAATG GACAG |
| 1601 | TGACT GTAGA AGATG GGCCA ACTAA ATaiG ATCCT CGGTG CCTGA CCCGC |
| 1651 | TATTA CTCTA GTTTC GTTAA TATGG AGAGA GATCT AGCTT CAGGA CTCAT |
| 1701 | TGGCC CTCTC CTCAT CTGCT ACAAA GAATC TGTAG ATCAA AGAGG AAACC |
| 1751 | AGATA ATGTC AGACA AGAGG AATGT CATCC TGTTT TCTGT ATTTG ATGAG |
| 1801 | AAGCG AAGCT GGTAC CTCAC AGAGA ATATA CAACG CTTTC TCCCC AATCC |
| 1851 | AGCTG GAGTG CAGCT TGAGG ATCCA GAGTT CCAAG CCTCC AACAT CATGC |
| 1901 | ACAGC ATCAA TGGCT ATGTT TTTGA TAGTT TGCAG TTGTC AGTTT GTTTG |
| 1951 | CATGA GGTGG CATAC TGGTA CATTC TAAGC ATTGG AGCAC AGACT GACTT |
| 2001 | CCTTT CTGTC TTCTT CTCTG GATAT ACCTT CAAAC ACAAA ATGGT CTATG |
| 2051 | AAGAC ACACT CACCC TATTC CCATT CTCAG GAGAA ACTGT CTTCA TGTCG |
| 2101 | ATGGA AAACC CAGGT CTATG GATTC TGGGG TGCCA CAACT CAGAC TTTCG |
| 2151 | GAACA GAGGC ATGAC CGCCT TACTG AAGGT TTCTA GTTGT GACAA GAACA |
| 2201 | CTGGT GATTA TTACG AGGAC AGTTA TGAAG ATATT TCAGC ATACT TGCTG |
| 2251 | AGTAA AAACA ATGCC ATTGA ACCAA GAAGC TTCTC TCAAA ACGGC GCGCC |
| 2301 | AGGTA CCTCA GAGTC TGCTA CCCCC GAGTC AGGGC CAGGA TCAGA GCCAG |
| 2351 | CCACC TCCGG GTCTG AGACA CCCGG GACTT CCGAG AGTGC CACCC CTGAG |
| 2401 | TCCGG ACCCG GGTCC GAGCC CGCCA CTTCC GGCTC CGAAA CTCCC GGCAC |
| 2451 | AAGCG AGAGC GCTAC CCCAG AGTCA GGACC AGGAA CATCT ACAGA GCCCT |
| 2501 | CTGAA GGCTC CGCTC CAGGG TCCCC AGCCG GCAGT CCCAC TAGCA CCGAG |
| 2551 | GAGGG AACCT CTGAA AGCGC CACAC CCGAA TCAGG GCCAG GGTCT GAGCC |
| 2601 | TGCTA CCAGC GGCAG CGAGA CACCA GGCAC CTCTG AGTCC GCCAC ACCAG |
| 2651 | AGTCC GGACC CGGAT CTCCC GCTGG GAGCC CCACC TCCAC TGAGG AGGGA |
| 2701 | TCTCC TGCTG GCTCT CCAAC ATCTA CTGAG GAAGG TACCT CAACC GAGCC |
| 2751 | ATCCG AGGGA TCAGC TCCCG GCACC TCAGA GTCGG CAACC CCGGA GTCTG |
| 2801 | GACCC GGAAC TTCCG AAAGT GCCAC ACCAG AGTCC GGTCC CGGGA CTTCA |
| 2851 | GAATC AGCAA CACCC GAGTC CGGCC CTGGG TCTGA ACCCG CCACA AGTGG |
| 2901 | TAGTG AGACA CCAGG ATCAG AACCT GCTAC CTCAG GGTCA GAGAC ACCCG |
| 2951 | GATCT CCGGC AGGCT CACCA ACCTC CACTG AGGAG GGCAC CAGCA CAGAA |
| 3001 | CCAAG CGAGG GCTCC GCACC CGGAA CAAGC ACTGA ACCCA GTGAG GGTTC |
| 3051 | AGCAC CCGGC TCTGA GCCGG CCACA AGTGG CAGTG AGACA CCCGG CACTT |
| 3101 | CAGAG AGTGC CACCC CCGAG AGTGG CCCAG GCACT AGTAC CGAGC CCTCT |
| 3151 | GAAGG CAGTG CGCCA GCCTC GAGCC CACCA GTCTT GAAAC GCCAT CAAGC |
| 3201 | TGAAA TAACT CGTAC TACTC TTCAG TCAGA TCAAG AGGAA ATCGA TTATG |
| 3251 | ATGAT ACCAT ATCAG TTGAA ATGAA GAAGG AAGAT TTTGA CATTT ATGAT |
| 3301 | GAGGA TGAAA ATCAG AGCCC CCGCA GCTTT CAAAA GAAAA CACGA CACTA |
| 3351 | TTTTA TTGCT GCAGT GGAGA GGCTC TGGGA TTATG GGATG AGTAG CTCCC |
| 3401 | CACAT GTTCT AAGAA ACAGG GCTCA GAGTG GCAGT GTCCC TCAGT TCAAG |
| 3451 | AAAGT TGTTT TCCAG GAATT TACTG ATGGC TCCTT TACTC AGCCC TTATA |
| 3501 | CCGTG GAGAA CTAAA TGAAC ATTTG GGACT CCTGG GGCCA TATAT AAGAG |
| 3551 | CAGAA GTTGA AGATA ATATC ATGGT AACTT TCAGA AATCA GGCCT CTCGT |
| 3601 | CCCTA TTCCT TCTAT TCTAG CCTTA TTTCT TATGA GGAAG ATCAG AGGCA |
| 3651 | AGGAG CAGAA CCTAG AAAAA ACTTT GTCAA GCCTA ATGAA ACCAA AACTT |
| 3701 | ACTTT TGGAA AGTGC AACAT CATAT GGCAC CCACT AAAGA TGAGT TTGAC |
| 3751 | TGCAA AGCCT GGGCT TATTT CTCTG ATGTT GACCT GGAAA AAGAT GTGCA |
| 3801 | CTCAG GCCTG ATTGG ACCCC TTCTG GTCTG CCACA CTAAC ACACT GAACC |
| 3851 | CTGCT CATGG GAGAC AAGTG ACAGT ACAGG AATTT GCTCT GTTTT TCACC |
| 3901 | ATCTT TGATG AGACC AAAAG CTGGT ACTTC ACTGA AAATA TGGAA AGAAA |
| 3951 | CTGCA GGGCT CCCTG CAATA TCCAG ATGGA AGATC CCACT TTTAA AGAGA |
| 4001 | ATTAT CGCTT CCATG CAATC AATGG CTACA TAATG GATAC ACTAC CTGGC |
| 4051 | TTAGT AATGG CTCAG GATCA AAGGA TTCGA TGGTA TCTGC TCAGC ATGGG |
| 4101 | CAGCA ATGAA AACAT CCATT CTATT CATTT CAGTG GACAT GTGTT CACTG |
| 4151 | TACGA AAAAA AGAGG AGTAT AAAAT GGCAC TGTAC AATCT CTATC CAGGT |
| 4201 | GTTTT TGAGA CAGTG GAAAT GTTAC CATCC AAAGC TGGAA TTTGG CGGGT |
| 4251 | GGAAT GCCTT ATTGG CGAGC ATCTA CATGC TGGGA TGAGC ACACT TTTTC |
| 4301 | TGGTG TACAG CAATA AGTGT CAGAC TCCCC TGGGA ATGGC TTCTG GACAC |
| 4351 | ATTAG AGATT TTCAG ATTAC AGCTT CAGGA CAATA TGGAC AGTGG GCCCC |
| 4401 | AAAGC TGGCC AGACT TCATT ATTCC GGATC AATCA ATGCC TGGAG CACCA |
| 4451 | AGGAG CCCTT TTCTT GGATC AAGGT GGATC TGTTG GCACC AATGA TTATT |
| 4501 | CACGG CATCA AGACC CAGGG TGCCC GTCAG AAGTT CTCCA GCCTC TACAT |
| 4551 | CTCTC AGTTT ATCAT CATGT ATAGT CTTGA TGGGA AGAAG TGGCA GACTT |
| 4601 | ATCGA GGAAA TTCCA CTGGA ACCTT AATGG TCTTC TTTGG CAATG TGGAT |
| 4651 | TCATC TGGGA TAAAA CACAA TATTT TTAAC CCTCC AATTA TTGCT CGATA |
| 4701 | CATCC GTTTG CACCC AACTC ATTAT AGCAT TCGCA GCACT CTTCG CATGG |
| 4751 | AGTTG ATGGG CTGTG ATTTA AATAG TTGCA GCATG CCATT GGGAA TGGAG |
| 4801 | AGTAA AGCAA TATCA GATGC ACAGA TTACT GCTTC ATCCT ACTTT ACCAA |
| 4851 | TATGT TTGCC ACCTG GTCTC CTTCA AAAGC TCGAC TTCAC CTCCA AGGGA |
| 4901 | GGAGT AATGC CTGGA GACCT CAGGT GAATA ATCCA AAAGA GTGGC TGCAA |
| 4951 | GTGGA CTTCC AGAAG ACAAT GAAAG TCACA GGAGT AACTA CTCAG GGAGT |
| 5001 | AAAAT CTCTG CTTAC CAGCA TGTAT GTGAA GGAGT TCCTC ATCTC CAGCA |
| 5051 | GTCAA GATGG CCATC AGTGG ACTCT CTTTT TTCAG AATGG CAAAG TAAAG |
| 5101 | GTTTT TCAGG GAAAT CAAGA CTCCT TCACA CCTGT GGTGA ACTCT CTAGA |
| 5151 | CCCAC CGTTA CTGAC TCGCT ACCTT CGAAT TCACC CCCAG AGTTG GGTGC |
| 5201 | ACCAG ATTGC CCTGA GGATG GAGGT TCTGG GCTGC GAGGC ACAGG ACCTC |
| 5251 | TACGA CAAAA CTCAC ACATG CCCAC CGTGC CCAGC TCCAG AACTC CTGGG |
| 5301 | CGGAC CGTCA GTCTT CCTCT TCCCC CCAAA ACCCA AGGAC ACCCT CATGA |
| 5351 | TCTCC CGGAC CCCTG AGGTC ACATG CGTGG TGGTG GACGT GAGCC ACGAA |
| 5401 | GACCC TGAGG TCAAG TTCAA CTGGT ACGTG GACGG CGTGG AGGTG CATAA |
| 5451 | TGCCA AGACA AAGCC GCGGG AGGAG CAGTA CAACA GCACG TACCG TGTGG |
| 5501 | TCAGC GTCCT CACCG TCCTG CACCA GGACT GGCTG AATGG CAAGG AGTAC |
| 5551 | AAGTG CAAGG TCTCC AACAA AGCCC TCCCA GCCCC CATCG AGAAA ACCAT |
| 5601 | CTCCA AAGCC AAAGG GCAGC CCCGA GAACC ACAGG TGTAC ACCCT GCCCC |
| 5651 | CATCC CGGGA TGAGC TGACC AAGAA CCAGG TCAGC CTGAC CTGCC TGGTC |
| 5701 | AAAGG CTTCT ATCCC AGCGA CATCG CCGTG GAGTG GGAGA GCAAT GGGCA |
| 5751 | GCCGG AGAAC AACTA CAAGA CCACG CCTCC CGTGT TGGAC TCCGA CGGCT |
| 5801 | CCTTC TTCCT CTACA GCAAG CTCAC CGTGG ACAAG AGCAG GTGGC AGCAG |
| 5851 | GGGAA CGTCT TCTCA TGCTC CGTGA TGCAT GAGGC TCTGC ACAAC CACTA |
| 5901 | CACGC AGAAG AGCCT CTCCC TGTCT CCGGG TAAAT GA |
| FVIII 169 protein sequence | |
| (SEQ ID NO: 88) | |
| 1 | MQIELSTCFF LCLLRFCFSA TRRYYLGAVE LSWDYMQSDL GELPVDARFP |
| 51 | PRVPKSFPFN TSVVYKKTLF VEFTDHLFNI AKPRPPWMGL LGPTIQAEVY |
| 101 | DTVVITLKNM ASHPVSLHAV GVSYWKASEG AEYDDQTSQR EKEDDKVFPG |
| 151 | GSHTYVWQVL KENGPMASDP LCLTYSYLSH VDLVKDLNSG LIGALLVCRE |
| 201 | GSLAKEKTQT LHKFILLFAV FDEGKSWHSE TKNSLMQDRD AASARAWPKM |
| 251 | HTVNGYVNRS LPGLIGCHRK SVYWHVIGMG TTPEVHSIFL EGHTFLVRNH |
| 301 | RQASLEISPI TFLTAQTLLM DLGQFLLFCH ISSHQHDGME AYVKVDSCPE |
| 351 | EPQLRMKNNE EAEDYDDDLT DSEMDVVRFD DDNSPSFIQI RSVAKKHPKT |
| 401 | WVHYIAAEEE DWDYAPLVLA PDDRSYKSQY LNNGPQRIGR KYKKVRFMAY |
| 451 | TDETFKTREA IQHESGILGP LLYGEVGDTL LIIFKNQASR PYNIYPHGIT |
| 501 | DVRPLYSRRL PKGVKHLKDF PILPGEIFKY KWTVTVEDGP TKSDPRCLTR |
| 551 | YYSSFVNMER DLASGLIGPL LICYKESVDQ RGNQIMSDKR NVILFSVFDE |
| 601 | NRSWYLTENI QRFLPNPAGV QLEDPEFQAS NIMHSINGYV FDSLQLSVCL |
| 651 | HEVAYWYILS IGAQTDFLSV FFSGYTFKHK MVYEDTLTLF PFSGETVFMS |
| 701 | MENPGLWILG CHNSDFRNRG MTALLKVSSC DKNTGDYYED SYEDISAYLL |
| 751 | SKNNAIEPRS FSQNGAPGTS ESATPESGPG SEPATSGSET PGTSESATPE |
| 801 | SGPGSEPATS GSETPGTSES ATPESGPGTS TEPSEGSAPG SPAGSPTSTE |
| 851 | EGTSESATPE SGPGSEPATS GSETPGTSES ATPESGPGSP AGSPTSTEEG |
| 901 | SPAGSPTSTE EGTSTEPSEG SAPGTSESAT PESGPGTSES ATPESGPGTS |
| 951 | ESATPESGPG SEPATSGSET PGSEPATSGS ETPGSPAGSP TSTEEGTSTE |
| 1001 | PSEGSAPGTS TEPSEGSAPG SEPATSGSET PGTSESATPE SGPGTSTEPS |
| 1051 | EGSAPASSPP VLKRHQAEIT RTTLQSDQEE IDYDDTISVE MKKEDFDIYD |
| 1101 | EDENQSPRSF QKKTRHYFIA AVERLWDYGM SSSPHVLRNR AQSGSVPQFK |
| 1151 | KVVFQEFTDG SFTQPLYRGE LNEHLGLLGP YIRAEVEDNI MVTFRNQASR |
| 1201 | PYSFYSSLIS YEEDQRQGAE PRKNFVKPNE TKTYFWKVQH HMAPTKDEFD |
| 1251 | CKAWAYFSDV DLEKDVHSGL IGPLLVCHTN TLNPAHGRQV TVQEFALFFT |
| 1301 | IFDETKSWYF TENMERNCRA PCNIQMEDPT FKENYRFHAI NGYIMDTLPG |
| 1351 | LVMAQDQRIR WYLLSMGSNE NIHSIHFSGH VFTVRKKEEY KMALYNLYPG |
| 1401 | VFETVEMLPS KAGIWRVECL IGEHLHAGMS TLFLVYSNKC QTPLGMASGH |
| 1451 | IRDFQITASG QYGQWAPKLA RLHYSGSINA WSTKEPFSWI KVDLLAPMII |
| 1501 | HGIKTQGARQ KFSSLYISQF IIMYSLDGKK WQTYRGNSTG TLMVFFGNVD |
| 1551 | SSGIKHNIFN PPIIARYIRL HPTHYSIRST LRMELMGCDL NSCSMPLGME |
| 1601 | SKAISDAQIT ASSYFTNMFA TWSPSKARLH LQGRSNAWRP QVNNPKEWLQ |
| 1651 | VDFQKTMKVT GVTTQGVKSL LTSMYVKEFL ISSSQDGHQW TLFFQNGKVK |
| 1701 | VFQGNQDSFT PVVNSLDPPL LTRYLRIHPQ SWVHQIALRM EVLGCEAQDL |
| 1751 | YDKTHTCPPC PAPELLGGPS VFLFPPKPKD TLMISRTPEV TCVVVDVSHE |
| 1801 | DPEVKFNWYV DGVEVHNAKT KPREEQYNST YRVVSVLTVL HQDWLNGKEY |
| 1851 | KCKVSNKALP APIEKTISKA KGQPREPQVY TLPPSRDELT KNQVSLTCLV |
| 1901 | KGFYPSDIAV EWESNGQPEN NYKTTPPVLD SDGSFFLYSK LTVDKSRWQQ |
| 1951 | GNVFSCSVMH EALHNHYTQK SLSLSPGK* |
| VWF034 nucleotide Sequence | |
| (SEQ ID NO: 91) | |
| 1 | ATGAT TCCTG CCAGA TTTGC CGGGG TGCTG CTTGC TCTGG CCCTC ATTTT |
| 51 | GCCAG GGACC CTTTG TGCAG AAGGA ACTCG CGGCA GGTCA TCCAC GGCCC |
| 101 | GATGC AGCCT TTTCG GAAGT GACTT CGTCA ACACC TTTGA TGGGA GCATG |
| 151 | TACAG CTTTG CGGGA TACTG CAGTT ACCTC CTGGC AGGGG GCTGC CAGAA |
| 201 | ACGCT CCTTC TCGAT TATTG GGGAC TTCCA GAATG GCAAG AGAGT GAGCC |
| 251 | TCTCC GTGTA TCTTG GGGAA TTTTT TGACA TCCAT TTGTT TGTCA ATGGT |
| 301 | ACCGT GACAC AGGGG GACCA AAGAG TCTCC ATGCC CTATG CCTCC AAAGG |
| 351 | GCTGT ATCTA GAAAC TGAGG CTGGG TACTA CAAGC TGTCC GGTGA GGCCT |
| 401 | ATGGC TTTGT GGCCA GGATC GATGG CAGCG GCAAC TTTCA AGTCC TGCTG |
| 451 | TCAGA CAGAT ACTTC AACAA GACCT GCGGG CTGTG TGGCA ACTTT AACAT |
| 501 | CTTTG CTGAA GATGA CTTTA TGACC CAAGA AGGGA CCTTG ACCTC GGACC |
| 551 | CTTAT GACTT TGCCA ACTCA TGGGC TCTGA GCAGT GGAGA ACAGT GGTGT |
| 601 | GAACG GGCAT CTCCT CCCAG CAGCT CATGC AACAT CTCCT CTGGG GAAAT |
| 651 | GCAGA AGGGC CTGTG GGAGC AGTGC CAGCT TCTGA AGAGC ACCTC GGTGT |
| 701 | TTGCC CGCTG CCACC CTCTG GTGGA CCCCG AGCCT TTTGT GGCCC TGTGT |
| 751 | GAGAA GACTT TGTGT GAGTG TGCTG GGGGG CTGGA GTGCG CCTGC CCTGC |
| 801 | CCTCC TGGAG TACGC CCGGA CCTGT GCCCA GGAGG GAATG GTGCT GTACG |
| 851 | GCTGG ACCGA CCACA GCGCG TGCAG CCCAG TGTGC CCTGC TGGTA TGGAG |
| 901 | TATAG GCAGT GTGTG TCCCC TTGCG CCAGG ACCTG CCAGA GCCTG CACAT |
| 951 | CAATG AAATG TGTCA GGAGC GATGC GTGGA TGGCT GCAGC TGCCC TGAGG |
| 1001 | GACAG CTCCT GGATG AAGGC CTCTG CGTGG AGAGC ACCGA GTGTC CCTGC |
| 1051 | GTGCA TTCCG GAAAG CGCTA CCCTC CCGGC ACCTC CCTCT CTCGA GACTG |
| 1101 | CAACA CCTGC ATTTG CCGAA ACAGC CAGTG GATCT GCAGC AATGA AGAAT |
| 1151 | GTCCA GGGGA GTGCC TTGTC ACTGG TCAAT CCCAC TTCAA GAGCT TTGAC |
| 1201 | AACAG ATACT TCACC TTCAG TGGGA TCTGC CAGTA CCTGC TGGCC CGGGA |
| 1251 | TTGCC AGGAC CACTC CTTCT CCATT GTCAT TGAGA CTGTC CAGTG TGCTG |
| 1301 | ATGAC CGCGA CGCTG TGTGC ACCCG CTCCG TCACC GTCCG GCTGC CTGGC |
| 1351 | CTGCA CAACA GCCTT GTGAA ACTGA AGCAT GGGGC AGGAG TTGCC ATGGA |
| 1401 | TGGCC AGGAC ATCCA GCTCC CCCTC CTGAA AGGTG ACCTC CGCAT CCAGC |
| 1451 | ATACA GTGAC GGCCT CCGTG CGCCT CAGCT ACGGG GAGGA CCTGC AGATG |
| 1501 | GACTG GGATG GCCGC GGGAG GCTGC TGGTG AAGCT GTCCC CCGTC TATGC |
| 1551 | CGGGA AGACC TGCGG CCTGT GTGGG AATTA CAATG GCAAC CAGGG CGACG |
| 1601 | ACTTC CTTAC CCCCT CTGGG CTGGC GGAGC CCCGG GTGGA GGACT TCGGG |
| 1651 | AACGC CTGGA AGCTG CACGG GGACT GCCAG GACCT GCAGA AGCAG CACAG |
| 1701 | CGATC CCTGC GCCCT CAACC CGCGC ATGAC CAGGT TCTCC GAGGA GGCGT |
| 1751 | GCGCG GTCCT GACGT CCCCC ACATT CGAGG CCTGC CATCG TGCCG TCAGC |
| 1801 | CCGCT GCCCT ACCTG CGGAA CTGCC GCTAC GACGT GTGCT CCTGC TCGGA |
| 1851 | CGGCC GCGAG TGCCT GTGCG GCGCC CTGGC CAGCT ATGCC GCGGC CTGCG |
| 1901 | CGGGG AGAGG CGTGC GCGTC GCGTG GCGCG AGCCA GGCCG CTGTG AGCTG |
| 1951 | AACTG CCCGA AAGGC CAGGT GTACC TGCAG TGCGG GACCC CCTGC AACCT |
| 2001 | GACCT GCCGC TCTCT CTCTT ACCCG GATGA GGAAT GCAAT GAGGC CTGCC |
| 2051 | TGGAG GGCTG CTTCT GCCCC CCAGG GCTCT ACATG GATGA GAGGG GGGAC |
| 2101 | TGCGT GCCCA AGGCC CAGTG CCCCT GTTAC TATGA CGGTG AGATC TTCCA |
| 2151 | GCCAG AAGAC ATCTT CTCAG ACCAT CACAO CATGT GCTAC TGTGA GGATG |
| 2201 | GCTTC ATGCA CTGTA CCATG AGTGG AGTCC CCGGA AGCTT GCTGC CTGAC |
| 2251 | GCTGT CCTCA GCAGT CCCCT GTCTC ATCGC AGCAA AAGGA GCCTA TCCTG |
| 2301 | TCGGC CCCCC ATGGT CAAGC TGGTG TGTCC CGCTG ACAAC CTGCG GGCTG |
| 2351 | AAGGG CTCGA GTGTA CCAAA ACGTG CCAGA ACTAT GACCT GGAGT GCATG |
| 2401 | AGCAT GGGCT GTGTC TCTGG CTGCC TCTGC CCCCC GGGCA TGGTC CGGCA |
| 2451 | TGAGA ACAGA TGTGT GGCCC TGGAA AGGTG TCCCT GCTTC CATCA GGGCA |
| 2501 | AGGAG TATGC CCCTG GAGAA ACAGT GAAGA TTGGC TGCAA CACTT GTGTC |
| 2551 | TGTCG GGACC GGAAG TGGAA CTGCA CAGAC CATGT GTGTG ATGCC ACGTG |
| 2601 | CTCCA CGATC GGCAT GGCCC ACTAC CTCAC CTTCG ACGGG CTCAA ATACC |
| 2651 | TGTTC CCCGG GGAGT GCCAG TACGT TCTGG TGCAG GATTA CTGCG GCAGT |
| 2701 | AACCC TGGGA CCTTT CGGAT CCTAG TGGGG AATAA GGGAT GCAGC CACCC |
| 2751 | CTCAG TGAAA TGCAA GAAAC GGGTC ACCAT CCTGG TGGAG GGAGG AGAGA |
| 2801 | TTGAG CTGTT TGACG GGGAG GTGAA TGTGA AGAGG CCCAT GAAGG ATGAG |
| 2851 | ACTCA CTTTG AGGTG GTGGA GTCTG GCCGG TACAT CATTC TGCTG CTGGG |
| 2901 | CAAAG CCCTC TCCGT GGTCT GGGAC CGCCA CCTGA GCATC TCCGT GGTCC |
| 2951 | TGAAG CAGAC ATACC AGGAG AAAGT GTGTG GCCTG TGTGG GAATT TTGAT |
| 3001 | GGCAT CCAGA ACAAT GACCT CACCA GCAGC AACCT CCAAG TGGAG GAAGA |
| 3051 | CCCTG TGGAC TTTGG GAACT CCTGG AAAGT GAGCT CGCAG TGTGC TGACA |
| 3101 | CCAGA AAAGT GCCTC TGGAC TCATC CCCTG CCACC TGCCA TAACA ACATC |
| 3151 | ATGAA GCAGA CGATG GTGGA TTCCT CCTGT AGAAT CCTTA CCAGT GACGT |
| 3201 | CTTCC AGGAC TGCAA CAACC TGGTG GACCC CGAGC CATAT CTGGA TGTCT |
| 3251 | GCATT TACGA CACCT GCTCC TGTGA GTCCA TTGGG GACTG CGCCG CATTC |
| 3301 | TGCGA CACCA TTGCT GCCTA TGCCC ACGTG TGTGC CCAGC ATGGC AAGGT |
| 3351 | GGTGA CCTGG AGGAC GGCCA CATTG TGCCC CCAGA GCTGC GAGGA GAGGA |
| 3401 | ATCTC CGGGA GAACG GGTAT GAGGC TGAGT GGCGC TATAA CAGCT GTGCA |
| 3451 | CCTGC CTGTC AAGTC ACGTG TCAGC ACCCT GAGCC ACTGG CCTGC CCTGT |
| 3501 | GCAGT GTGTG GAGGG CTGCC ATGCC CACTG CCCTC CAGGG AAAAT CCTGG |
| 3551 | ATGAG CTTTT GCAGA CCTGC GTTGA CCCTG AAGAC TGTCC AGTGT GTGAG |
| 3601 | GTGGC TGGCC GGCGT TTTGC CTCAG GAAAG AAAGT CACCT TGAAT CCCAG |
| 3651 | TGACC CTGAG CACTG CCAGA TTTGC CACTG TGATG TTGTC AACCT CACCT |
| 3701 | GTGAA GCCTG CCAGG AGCCG ATATC GGGTA CCTCA GAGTC TGCTA CCCCC |
| 3751 | GAGTC AGGGC CAGGA TCAGA GCCAG CCACC TCCGG GTCTG AGACA CCCGG |
| 3801 | GACTT CCGAG AGTGC CACCC CTGAG TCCGG ACCCG GGTCC GAGCC CGCCA |
| 3851 | CTTCC GGCTC CGAAA CTCCC GGCAC AAGCG AGAGC GCTAC CCCAG AGTCA |
| 3901 | GGACC AGGAA CATCT ACAGA GCCCT CTGAA GGCTC CGCTC CAGGG TCCCC |
| 3951 | AGCCG GCAGT CCCAC TAGCA CCGAG GAGGG AACCT CTGAA AGCGC CACAC |
| 4001 | CCGAA TCAGG GCCAG GGTCT GAGCC TGCTA CCAGC GGCAG CGAGA CACCA |
| 4051 | GGCAC CTCTG AGTCC GCCAC ACCAG AGTCC GGACC CGGAT CTCCC GCTGG |
| 4101 | GAGCC CCACC TCCAC TGAGG AGGGA TCTCC TGCTG GCTCT CCAAC ATCTA |
| 4151 | CTGAG GAAGG TACCT CAACC GAGCC ATCCG AGGGA TCAGC TCCCG GCACC |
| 4201 | TCAGA GTCGG CAACC CCGGA GTCTG GACCC GGAAC TTCCG AAAGT GCCAC |
| 4251 | ACCAG AGTCC GGTCC CGGGA CTTCA GAATC AGCAA CACCC GAGTC CGGCC |
| 4301 | CTGGG TCTGA ACCCG CCACA AGTGG TAGTG AGACA CCAGG ATCAG AACCT |
| 4351 | GCTAC CTCAG GGTCA GAGAC ACCCG GATCT CCGGC AGGCT CACCA ACCTC |
| 4401 | CACTG AGGAG GGCAC CAGCA CAGAA CCAAG CGAGG GCTCC GCACC CGGAA |
| 4451 | CAAGC ACTGA ACCCA GTGAG GGTTC AGCAC CCGGC TCTGA GCCGG CCACA |
| 4501 | AGTGG CAGTG AGACA CCCGG CACTT CAGAG AGTGC CACCC CCGAG AGTGG |
| 4551 | CCCAG GCACT AGTAC CGAGC CCTCT GAAGG CAGTG CGCCA GATTC TGGCG |
| 4601 | GTGGA GGTTC CGGTG GCGGG GGATC CGGTG GCGGG GGATC CGGTG GCGGG |
| 4651 | GGATC CGGTG GCGGG GGATC CCTGG TCCCC CGGGG CAGCG GAGGC GACAA |
| 4701 | AACTC ACACA TGCCC ACCGT GCCCA GCTCC AGAAC TCCTG GGCGG ACCGT |
| 4751 | CAGTC TTCCT CTTCC CCCCA AAACC CAAGG ACACC CTCAT GATCT CCCGG |
| 4801 | ACCCC TGAGG TCACA TGCGT GGTGG TGGAC GTGAG CCACG AAGAC CCTGA |
| 4851 | GGTCA AGTTC AACTG GTACG TGGAC GGCGT GGAGG TGCAT AATGC CAAGA |
| 4901 | CAAAG CCGCG GGAGG AGCAG TACAA CAGCA CGTAC CGTGT GGTCA GCGTC |
| 4951 | CTCAC CGTCC TGCAC CAGGA CTGGC TGAAT GGCAA GGAGT ACAAG TGCAA |
| 5001 | GGTCT CCAAC AAAGC CCTCC CAGCC CCCAT CGAGA AAACC ATCTC CAAAG |
| 5051 | CCAAA GGGCA GCCCC GAGAA CCACA GGTGT ACACC CTGCC CCCAT CCCGG |
| 5101 | GATGA GCTGA CCAAG AACCA GGTCA GCCTG ACCTG CCTGG TCAAA GGCTT |
| 5151 | CTATC CCAGC GACAT CGCCG TGGAG TGGGA GAGCA ATGGG CAGCC GGAGA |
| 5201 | ACAAC TACAA GACCA CGCCT CCCGT GTTGG ACTCC GACGG CTCCT TCTTC |
| 5251 | CTCTA CAGCA AGCTC ACCGT GGACA AGAGC AGGTG GCAGC AGGGG AACGT |
| 5301 | CTTCT CATGC TCCGT GATGC ATGAG GCTCT GCACA ACCAC TACAC GCAGA |
| 5351 | AGAGC CTCTC CCTGT CTCCG GGTAA ATGA |
| VWF034 Protein Sequence | |
| (SEQ ID NO: 92) | |
| 1 | MIPARFAGVL LALALILPGT LCAEGTRGRS STARCSLFGS DFVNTFDGSM |
| 51 | YSFAGYCSYL LAGGCQKRSF SIIGDFQNGK RVSLSVYLGE FFDIHLFVNG |
| 101 | TVTQGDQRVS MPYASKGLYL ETEAGYYKLS GEAYGFVARI DGSGNFQVLL |
| 151 | SDRYFNKTCG LCGNFNIFAE DDFMTQEGTL TSDPYDFANS WALSSGEQWC |
| 201 | ERASPPSSSC NISSGEMQKG LWEQCQLLKS TSVFARCHPL VDPEPFVALC |
| 251 | EKTLCECAGG LECACPALLE YARTCAQEGM VLYGWTDHSA CSPVCPAGME |
| 301 | YRQCVSPCAR TCQSLHINEM CQERCVDGCS CPEGQLLDEG LCVESTECPC |
| 351 | VHSGKRYPPG TSLSRDCNTC ICRNSQWICS NEECPGECLV TGQSHFKSFD |
| 401 | NRYFTFSGIC QYLLARDCQD HSFSIVIETV QCADDRDAVC TRSVTVPLPG |
| 451 | LHNSLVKLKH GAGVAMDGQD IQLPLLKGDL RIQHTVTASV RLSYGEDLQM |
| 501 | DWDGRGRLLV KLSPVYAGKT CGLCGNYNGN QGDDFLTPSG LAEPRVEDFG |
| 551 | NAWKLHGDCQ DLQKQHSDPC ALNPRMTRFS EEACAVLTSP TFEACHRAVS |
| 601 | PLPYLRNCRY DVCSCSDGRE CLCGALASYA AACAGRGVRV AWREPGRCEL |
| 651 | NCPKGQVYLQ CGTPCNLTCR SLSYPDEECN EACLEGCFCP PGLYMDERGD |
| 701 | CVPKAQCPCY YDGEIFQPED IFSDHHTMCY CEDGFMHCTM SGVPGSLLPD |
| 751 | AVLSSPLSHR SKRSLSCRPP MVKLVCPADN LRAEGLECTK TCQNYDLECM |
| 801 | SMGCVSGCLC PPGMVRHENR CVALERCPCF HQGKEYAPGE TVKIGCNTCV |
| 851 | CRDRKWNCTD HVCDATCSTI GMAHYLTFDG LKYLFPGECQ YVLVQDYCGS |
| 901 | NPGTFRILVG NKGCSHPSVK CKKRVTILVE GGEIELFDGE VNVKRPMKDE |
| 951 | THFEVVESGR YIILLLGKAL SVVWDRHLSI SVVLKQTYQE KVCGLCGNFD |
| 1001 | GIQNNDLTSS NLQVEEDPVD FGNSWKVSSQ CADTRKVPLD SSPATCHNNI |
| 1051 | MKQTMVDSSC RILTSDVFQD CNKLVDPEPY LDVCIYDTCS CESIGDCAAF |
| 1101 | CDTIAAYAHV CAQHGKVVTW RTATLCPQSC EERNLRENGY EAEWRYNSCA |
| 1151 | PACQVTCQHP EPLACPVQCV EGCHAHCPPG KILDELLQTC VDPEDCPVCE |
| 1201 | VAGRRFASGK KVTLNPSDPE HCQICHCDVV NLTCEACQEP ISGTSESATP |
| 1251 | ESGPGSEPAT SGSETPGTSE SATPESGPGS EPATSGSETP GTSESATPES |
| 1301 | GPGTSTEPSE GSAPGSPAGS PTSTEEGTSE SATPESGPGS EPATSGSETP |
| 1351 | GTSESATPES GPGSPAGSPT STEEGSPAGS PTSTEEGTST EPSEGSAPGT |
| 1401 | SESATPESGP GTSESATPES GPGTSESATP ESGPGSEPAT SGSETPGSEP |
| 1451 | ATSGSETPGS PAGSPTSTEE GTSTEPSEGS APGTSTEPSE GSAPGSEPAT |
| 1501 | SGSETPGTSE SATPESGPGT STEPSEGSAP DIGGGGGSGG GGSLVPRGSG |
| 1551 | GDKTHTCPPC PAPELLGGPS VFLFPPKPKD TLMISRTPEV TCVVVDVSHE |
| 1601 | DPEVKFNWYV DGVEVHNAKT KPREEQYNST YRVVSVLTVL HQDWLNGKEY |
| 1651 | KCKVSNKALP APIEKTISKA KGQPREPQVY TLPPSRDELT KNQVSLTCLV |
| 1701 | KGFYPSDIAV EWESNGQPEN NYKTTPPVLD SDGSFFLYSK LTVDKSRWQQ |
| 1751 | GNVFSCSVMH EALHNHYTQK SLSLSPGK* |

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are incorporated by reference herein in their entirety.

1. A chimeric molecule comprising a von Willebrand Factor (VWF) protein, a heterologous moiety (H1), an extended recombinant polypeptide (XTEN) sequence, and a VWF linker connecting the VWF protein with the heterologous moiety, wherein the VWF linker comprises a polypeptide selected from:
i. an a2 region from Factor VIII (FVIII);
ii. an a1 region from FVIII;
iii. an a3 region from FVIII;
iv. a thrombin cleavage site which comprises X-V-P-R (SEQ ID NO: 3) and a PAR1 exosite interaction motif, wherein X is an aliphatic amino acid; or
v. any combination thereof, and
wherein the XTEN sequence is connected to the VWF protein, the heterologous moiety (H1), the VWF linker, or any combination thereof.

2. The chimeric molecule of claim 1, wherein the XTEN sequence connects the VWF protein with the VWF linker or the VWF linker with the heterologous moiety.

3. The chimeric molecule of claim 1, further comprising a second polypeptide chain which comprises a FVIII protein, wherein the first polypeptide chain comprising the VWF protein and the second polypeptide chain are associated with each other.

4. The chimeric molecule of claim 3, wherein the FVIII protein further comprises an additional XTEN sequence.

5. (canceled)

6. The chimeric molecule of claim 3, wherein the second polypeptide chain further comprises a second heterologous moiety (H2).

7. A chimeric molecule comprising a first polypeptide chain which comprises a VWF protein, a heterologous moiety (H1), and a VWF linker connecting the VWF protein and the heterologous moiety (H1) and a second polypeptide chain comprising a FVIII protein and an XTEN sequence, wherein the VWF linker in the first polypeptide chain comprises:
i. an a2 region from FVIII;
ii. an a1 region from FVIII;
iii. an a3 region from FVIII;
iv. a thrombin cleavage site which comprises X-V-P-R (SEQ ID NO: 3) and a PAR1 exosite interaction motif, wherein X is an aliphatic amino acid; or
v. any combination thereof,
and wherein the first polypeptide chain and the second polypeptide chain are associated with each other.

8-11. (canceled)

12. The chimeric molecule of claim 1, wherein the XTEN sequence comprises about 42 amino acids, about 72 amino acids, about 108 amino acids, about 144 amino acids, about 180 amino acids, about 216 amino acids, about 252 amino acids, about 288 amino acids, about 324 amino acids, about 360 amino acids, about 396 amino acids, about 432 amino acids, about 468 amino acids, about 504 amino acids, about 540 amino acids, about 576 amino acids, about 612 amino acids, about 624 amino acids, about 648 amino acids, about 684 amino acids, about 720 amino acids, about 756 amino acids, about 792 amino acids, about 828 amino acids, about 836 amino acids, about 864 amino acids, about 875 amino acids, about 912 amino acids, about 923 amino acids, about 948 amino acids, about 1044 amino acids, about 1140 amino acids, about 1236 amino acids, about 1318 amino acids, about 1332 amino acids, about 1428 amino acids, about 1524 amino acids, about 1620 amino acids, about 1716 amino acids, about 1812 amino acids, about 1908 amino acids, or about 2004 amino acids.

13. The chimeric molecule of claim 1, wherein the XTEN sequence is selected from AE42, AE72, AE864, AE576, AE288, AE144, AG864, AG576, AG288, AG144, SEQ ID NO: 39; SEQ ID NO: 40; SEQ ID NO: 47; SEQ ID NO: 45; SEQ ID NO: 44; SEQ ID NO: 41; SEQ ID NO: 48; SEQ ID NO: 46, SEQ ID NO: 44, and SEQ ID NO: 42.

14-17. (canceled)

18. The chimeric molecule of claim 1, wherein:
(i) the VWF linker comprises the a2 region which comprises an amino acid sequence at least about 80%, about 85%, about 90%, about 95%, or 100% identical to Glu720 to Arg740 corresponding to full-length FVIII, wherein the a2 region is capable of being cleaved by thrombin;
(ii) the VWF linker comprises the a1 region which comprises an amino acid sequence at least about 80%, at least about 85%, at least about 90%, at least about 95%, or 100% identical to Met337 to Arg372 corresponding to full-length FVIII, wherein the a1 region is capable of being cleaved by thrombin;
(ii) the VWF linker comprises the a3 region which comprises an amino acid sequence at least about 80%, about 85%, about 90%, about 95%, or 100% identical to Glu1649 to Arg1689 corresponding to full-length FVIII, wherein the a3 region is capable of being cleaved by thrombin;
(iv) the VWF linker comprises the thrombin cleavage site which comprises X-V-P-R (SEQ ID NO: 3) and the PAR1 exosite interaction motif and wherein the PAR1 exosite interaction motif comprises S-F-L-L-R-N(SEQ ID NO: 7); or
(v) any combination thereof.

19. The chimeric molecule of claim 18, wherein:
(i) the a2 region comprises SEQ ID NO: 4;
(ii) the a1 region comprises SEQ ID NO: 5;
(iii) the a3 region comprises SEQ ID NO: 6; or
(iv) any combination thereof.

20-24. (canceled)

25. The chimeric molecule of claim 1, wherein the PAR1 exosite interaction motif further comprises a sequence selected from the group consisting of P, P-N, P-N-D, P-N-D-K (SEQ ID NO: 8), P-N-D-K-Y (SEQ ID NO: 9), P-N-D-K-Y-E (SEQ ID NO: 10), P-N-D-K-Y-E-P (SEQ ID NO: 11), P-N-D-K-Y-E-P-F (SEQ ID NO: 12), P-N-D-K-Y-E-P-F-W (SEQ ID NO: 13), P-N-D-K-Y-E-P-F-W-E (SEQ ID NO: 14), P-N-D-K-Y-E-P-F-W-E-D (SEQ ID NO: 20), P-N-D-K-Y-E-P-F-W-E-D-E (SEQ ID NO: 21), P-N-D-K-Y-E-P-F-W-E-D-E-E (SEQ ID NO: 22), P-N-D-K-Y-E-P-F-W-E-D-E-E-S(SEQ ID NO: 23), and any combination thereof.

26-29. (canceled)

30. The chimeric molecule of claim 1, wherein the VWF linker further comprises one or more amino acids, wherein the one or more amino acids comprise GlyGly or a Gly/Ser peptide.

31-35. (canceled)

36. The chimeric molecule of claim 1, wherein the VWF protein comprises the D′ domain and D3 domain of VWF, wherein the D′ domain and D3 domain are capable of binding to a FVIII protein.

37-43. (canceled)

44. The chimeric molecule of claim 36, wherein the VWF protein consists essentially of or consists of: (1) the D′ and D3 domains of VWF or fragments thereof; (2) the D1, D′, and D3 domains of VWF or fragments thereof; (3) the D2, D′, and D3 domains of VWF or fragments thereof; (4) the D1, D2, D′, and D3 domains of VWF or fragments thereof; or (5) the D1, D2, D′, D3, and A1 domains of VWF or fragments thereof.

45. (canceled)

46. The chimeric molecule of claim 1, wherein the VWF protein is pegylated, glycosylated, hesylated, or polysialylated.

47. (canceled)

48. The chimeric molecule of claim 1, wherein the heterologous moiety (H1) comprises an immunoglobulin constant region or a portion thereof, albumin, albumin-binding polypeptide, PAS, the C-terminal peptide (CTP) of the β subunit of human chorionic gonadotropin, polyethylene glycol (PEG), hydroxyethyl starch (HES), albumin-binding small molecules, or any combinations thereof, or wherein the heterologous moiety (H1) comprises a clearance receptor, or fragment thereof, wherein the clearance receptor blocks binding of a FVIII protein to FVIII clearance receptors.

49. The chimeric molecule of claim 48, wherein the immunoglobulin constant region or a portion thereof comprises an FcRn binding partner.

50-52. (canceled)

53. The chimeric molecule of claim 6, wherein the second heterologous moiety (H2) comprises an immunoglobulin constant region or a portion thereof, albumin, albumin-binding polypeptide, PAS, the C-terminal peptide (CTP) of the β subunit of human chorionic gonadotropin, polyethylene glycol (PEG), hydroxyethyl starch (HES), albumin-binding small molecules, or any combinations thereof.

54-56. (canceled)

57. The chimeric molecule of claim 6, wherein the second heterologous moiety comprises an FcRn binding partner.

58-61. (canceled)

62. The chimeric molecule of claim 6, wherein the first heterologous moiety is an FcRn binding partner and the second heterologous moiety is an FcRn binding partner.

63-67. (canceled)

68. The chimeric molecule of claim 6, comprising a formula selected from the group consisting of:
(a) V-L1-X1-H1:H2-L2-X2-C;
(b) V-X1-L1-H1:H2-L2-X2-C;
(c) V-L1-X1-H1:H2-X2-L2-C;
(d) V-X1-L1-H1:H2-X2-L2-C;
(e) V-L1-X1-H1:H2-L2-C(X2);
(f) V-X1-L1-H1:H2-L2-C(X2);
(g) C-X2-L2-H2:H1-X1-L1-V;
(h) C-X2-L2-H2:H1-L1-X1-V;
(i) C-L2-X2-H2:H1-L1-X1-V;
(j) C-L2-X2-H2:H1-L1-X1-V;
(k) C(X2)-L2-H2:H1-X1-L1-V; or
(l) C(X2)-L2-H2:H1-L1-X1-V;
wherein V is the VWF protein;
L1 is the VWF linker;
L2 is an optional FVIII linker;
H1 is the first heterologous moiety;
H2 is the second heterologous moiety;
C is the FVIII protein;
C(X2) is the FVIII protein fused to the XTEN sequence, wherein the XTEN sequence is inserted between two FVIII amino acids adjacent to each other;
(-) is a peptide bond or one or more amino acids; and
(:) is a covalent bond between the H1 and the H2.

69-83. (canceled)

84. A polynucleotide or a set of polynucleotides encoding the chimeric molecule of claim 1 or a complementary sequence thereof.

85. (canceled)

86. A vector or a set of vectors comprising the polynucleotide or the set of polynucleotides of claim 84 and one or more promoter operably linked to the polynucleotide or the set of polynucleotides.

87. (canceled)

88. A host cell comprising the polynucleotide or the set of the polynucleotides of claim 86.

89-90. (canceled)

91. A pharmaceutical composition comprising the chimeric molecule of claim 1 and a pharmaceutically acceptable carrier.

92-93. (canceled)

94. A method of reducing a frequency or degree of or preventing an occurrence of a bleeding episode in a subject in need thereof comprising administering an effective amount of the chimeric molecule of claim 1.

95-97. (canceled)

98. A method of making a chimeric molecule, comprising transfecting one or more host cell with the polynucleotide or the set of polynucleotides of claim 84 and expressing the chimeric molecule in the host cell.

99. (canceled)

100. A method of improving FVIII activity of a chimeric FVIII protein comprising a VWF protein, a heterologous moiety (H1), and a VWF linker connecting the VWF protein and the heterologous moiety (H1) and a second polypeptide chain comprising a FVIII protein and an XTEN sequence, wherein the VWF linker in the first polypeptide chain comprises:
vi. an a2 region from FVIII;
vii. an a1 region from FVIII;
viii. an a3 region from FVIII;
ix. a thrombin cleavage site which comprises X-V-P-R (SEQ ID NO: 3) and a PAR1 exosite interaction motif, wherein X is an aliphatic amino acid; or
x. any combination thereof.

101-111. (canceled)


SEQUENCE LISTING (92 sequences)

SEQ ID NO: 1 - DNA, 8442 bp, Homo sapiens
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcrgagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgccccccgacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgygtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagstgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtcttgctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtga

SEQ ID NO: 2 - protein, 2813 aa, Homo sapiens
  MISC_FEATURE (2016)..(2016): where Xaa can be any amino acid other than cysteine
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVEXHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 3 - protein, 4 aa, Artificial Sequence
  Thrombin cleavage site
XVPR

SEQ ID NO: 4 - protein, 34 aa, Artificial Sequence
  a2 region
ISDKNTGDYYEDSYEDISAYLLSKNNAIEPRSFS

SEQ ID NO: 5 - protein, 40 aa, Artificial Sequence
  a1 region
ISMKNNEEAEDYDDDLTDSEMDVVRFDDDNSPSFIQIRSV

SEQ ID NO: 6 - protein, 46 aa, Artificial Sequence
  a3 region
ISEITRTTLQSDQEEIDYDDTISVEMKKEDFDIYDEDENQSPRSFQ

SEQ ID NO: 7 - protein, 6 aa, Artificial Sequence
  PAR1 exosite interaction motif
SFLLRN

SEQ ID NO: 8 - protein, 4 aa, Artificial Sequence
  PAR1 exosite interaction motif
PNDK

SEQ ID NO: 9 - protein, 5 aa, Artificial Sequence
  PAR1 exosite interaction motif
PNDKY

SEQ ID NO: 10 - protein, 6 aa, Artificial Sequence
  PAR1 exosite interaction motif
PNDKYE

SEQ ID NO: 11 - protein, 7 aa, Artificial Sequence
  PAR1 exosite interaction motif
PNDKYEP

SEQ ID NO: 12 - protein, 8 aa, Artificial Sequence
  PAR1 exosite interaction motif
PNDKYEPF

SEQ ID NO: 13 - protein, 9 aa, Artificial Sequence
  PAR1 exosite interaction motif
PNDKYEPFW

SEQ ID NO: 14 - protein, 10 aa, Artificial Sequence
  PAR1 exosite interaction motif
PNDKYEPFWE

SEQ ID NO: 15 - protein, 19 aa, Artificial Sequence
  Signal Peptide
MQIELSTCFFLCLLRFCFS

SEQ ID NO: 16 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 17 - DNA, 7053 bp, Homo sapiens
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 18 - protein, 1438 aa, Artificial Sequence
  B-domain-deleted FVIII
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 19 - DNA, 4371 bp, Artificial Sequence
  B-domain-deleted FVIII
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctac

SEQ ID NO: 20 - protein, 11 aa, Artificial Sequence
  PAR1 exosite interaction motif
PNDKYEPFWED

SEQ ID NO: 21 - protein, 12 aa, Artificial Sequence
  PAR1 exosite interaction motif
PNDKYEPFWEDE

SEQ ID NO: 22 - protein, 13 aa, Artificial Sequence
  PAR1 exosite interaction motif
PNDKYEPFWEDEE

SEQ ID NO: 23 - protein, 14 aa, Artificial Sequence
  PAR1 exosite interaction motif
PNDKYEPFWEDEES

SEQ ID NO: 24 - protein, 26 aa, Artificial Sequence
  VWF linker
GGLVPRSFLLRNPNDKYEPFWEDEES

SEQ ID NO: 25 - protein, 4 aa, Artificial Sequence
  Thrombin cleavage site
LVPR

SEQ ID NO: 26 - protein, 7 aa, Artificial Sequence
  Thrombin cleavage site
ALRPRVV

SEQ ID NO: 27 - protein, 9 aa, Artificial Sequence
  FXIa cleavage site
TQSFNDFTR

SEQ ID NO: 28 - protein, 10 aa, Artificial Sequence
  FXIa cleavage site
SVSQTSKLTR

SEQ ID NO: 29 - protein, 10 aa, Artificial Sequence
  Thrombin cleavage site
DFLAEGGGVR

SEQ ID NO: 30 - protein, 7 aa, Artificial Sequence
  Thrombin cleavage site
TTKIKPR

SEQ ID NO: 31 - protein, 5 aa, Artificial Sequence
  Thrombin cleavage site
LVPRG

SEQ ID NO: 32 - protein, 20 aa, Artificial Sequence
  PAS sequence
ASPAAPAPASPAAPAPSAPA

SEQ ID NO: 33 - protein, 20 aa, Artificial Sequence
  PAS sequence
AAPASPAPAAPSAPAPAAPS

SEQ ID NO: 34 - protein, 20 aa, Artificial Sequence
  PAS sequence
APSSPSPSAPSSPSPASPSS

SEQ ID NO: 35 - protein, 19 aa, Artificial Sequence
  PAS sequence
APSSPSPSAPSSPSPASPS

SEQ ID NO: 36 - protein, 20 aa, Artificial Sequence
  PAS sequence
SSPSAPSPSSPASPSPSSPA

SEQ ID NO: 37 - protein, 24 aa, Artificial Sequence
  PAS sequence
AASPAAPSAPPAAASPAAPSAPPA

SEQ ID NO: 38 - protein, 20 aa, Artificial Sequence
  PAS sequence
ASAAAPAAASAAASAPSAAA

SEQ ID NO: 39 - protein, 42 aa, Artificial Sequence
  XTEN AE42
GAPGSPAGSPTSTEEGTSESATPESGPGSEPATSGSETPASS

SEQ ID NO: 40 - protein, 78 aa, Artificial Sequence
  XTEN AE72
GAPTSESATPESGPGSEPATSGSETPGTSESATPESGPGSEPATSGSETPGTSESATPESGPGTSTEPSEGSAPGASS

SEQ ID NO: 41 - protein, 143 aa, Artificial Sequence
  XTEN AE144
GSEPATSGSETPGTSESATPESGPGSEPATSGSETPGSPAGSPTSTEEGTSTEPSEGSAPGSEPATSGSETPGSEPATSGSETPGSEPATSGSETPGTSTEPSEGSAPGTSESAPESGPGSEPATSGSETPGTSTEPSEGSAP

SEQ ID NO: 42 - protein, 144 aa, Artificial Sequence
  XTEN AG144
GTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSP

SEQ ID NO: 43 - protein, 288 aa, Artificial Sequence
  XTEN AE288
GTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 44 - protein, 288 aa, Artificial Sequence
  XTEN AG288
PGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGS

SEQ ID NO: 45 - protein, 576 aa, Artificial Sequence
  XTEN AE576
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAP

SEQ ID NO: 46 - protein, 576 aa, Artificial Sequence
  XTEN AG576
PGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGS

SEQ ID NO: 47 - protein, 864 aa, Artificial Sequence
  XTEN AE864
GSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAP

SEQ ID NO: 48 - protein, 864 aa, Artificial Sequence
  XTEN AG864
GASPGTSSTGSPGSSPSASTGTGPGSSPSASTGTGPGTPGSGTASSSPGSSTPSGATGSPGSSPSASTGTGPGASPGTSSTGSPGTPGSGTASSSPGSSTPSGATGSPGTPGSGTASSSPGASPGTSSTGSPGASPGTSSTGSPGTPGSGTASSSPGSSTPSGATGSPGASPGTSSTGSPGTPGSGTASSSPGSSTPSGATGSPGSSPSASTGTGPGSSPSASTGTGPGSSTPSGATGSPGSSTPSGATGSPGASPGTSSTGSPGASPGTSSTGSPGASPGTSSTGSPGTPGSGTASSSPGASPGTSSTGSPGASPGTSSTGSPGASPGTSSTGSPGSSPSASTGTGPGTPGSGTASSSPGASPGTSSTGSPGASPGTSSTGSPGASPGTSSTGSPGSSTPSGATGSPGSSTPSGATGSPGASPGTSSTGSPGTPGSGTASSSPGSSTPSGATGSPGSSTPSGATGSPGSSTPSGATGSPGSSPSASTGTGPGASPGTSSTGSPGASPGTSSTGSPGTPGSGTASSSPGASPGTSSTGSPGASPGTSSTGSPGASPGTSSTGSPGASPGTSSTGSPGTPGSGTASSSPGSSTPSGATGSPGTPGSGTASSSPGSSTPSGATGSPGTPGSGTASSSPGSSTPSGATGSPGSSTPSGATGSPGSSPSASTGTGPGSSPSASTGTGPGASPGTSSTGSPGTPGSGTASSSPGSSTPSGATGSPGSSPSASTGTGPGSSPSASTGTGPGASPGTSSTGSPGASPGTSSTGSPGSSTPSGATGSPGSSPSASTGTGPGASPGTSSTGSPGSSPSASTGTGPGTPGSGTASSSPGSSTPSGATGSPGSSTPSGATGSPGASPGTSSTGSP

SEQ ID NO: 49 - protein, 12 aa, Artificial Sequence
  XTEN Motif Family AD
GESPGGSSGSES

SEQ ID NO: 50 - protein, 12 aa, Artificial Sequence
  XTEN Motif Family AD
GSEGSSGPGESS

SEQ ID NO: 51 - protein, 12 aa, Artificial Sequence
  XTEN Motif Family AD
GSSESGSSEGGP

SEQ ID NO: 52 - protein, 12 aa, Artificial Sequence
  XTEN Motif Family AD
GSGGEPSESGSS

SEQ ID NO: 53 - protein, 12 aa, Artificial Sequence
  XTEN Motif Family AE, AM
GSPAGSPTSTEE

SEQ ID NO: 54 - protein, 12 aa, Artificial Sequence
  XTEN Motif Family AE, AM, AQ
GSEPATSGSETP

SEQ ID NO: 55 - protein, 12 aa, Artificial Sequence
  XTEN Motif Family AE, AM, AQ
GTSESATPESGP

SEQ ID NO: 56 - protein, 12 aa, Artificial Sequence
  XTEN Motif Family AE, AM, AQ
GTSTEPSEGSAP

SEQ ID NO: 57 - protein, 12 aa, Artificial Sequence
  XTEN Motif Family AF, AM
GSTSESPSGTAP

SEQ ID NO: 58 - protein, 12 aa, Artificial Sequence
  XTEN Motif Family AF, AM
GTSTPESGSASP

SEQ ID NO: 59 - protein, 12 aa, Artificial Sequence
  XTEN Motif Family AF, AM
GTSPSGESSTAP

SEQ ID NO: 60 - protein, 12 aa, Artificial Sequence
  XTEN Motif Family AF, AM
GSTSSTAESPGP

SEQ ID NO: 61 - protein, 12 aa, Artificial Sequence
  XTEN Motif Family AG, AM
GTPGSGTASSSP

SEQ ID NO: 62 - protein, 12 aa, Artificial Sequence
  XTEN Motif Family AG, AM
GSSTPSGATGSP

SEQ ID NO: 63 - protein, 12 aa, Artificial Sequence
  XTEN Motif Family AG, AM
GSSPSASTGTGP

SEQ ID NO: 64 - protein, 12 aa, Artificial Sequence
  XTEN Motif Family AG, AM
GASPGTSSTGSP

SEQ ID NO: 65 - protein, 12 aa, Artificial Sequence
  XTEN Motif Family AQ
GEPAGSPTSTSE

SEQ ID NO: 66 - protein, 12 aa, Artificial Sequence
  XTEN Motif Family AQ
GTGEPSSTPASE

SEQ ID NO: 67 - protein, 12 aa, Artificial Sequence
  XTEN Motif Family AQ
GSGPSTESAPTE

SEQ ID NO: 68 - protein, 12 aa, Artificial Sequence
  XTEN Motif Family AQ
GSETPSGPSETA

SEQ ID NO: 69 - protein, 12 aa, Artificial Sequence
  XTEN Motif Family AQ
GPSETSTSEPGA

SEQ ID NO: 70 - protein, 12 aa, Artificial Sequence
  XTEN Motif Family AQ
GSPSEPTEGTSA

SEQ ID NO: 71 - protein, 12 aa, Artificial Sequence
  XTEN Motif Family BC
GSGASEPTSTEP

SEQ ID NO: 72 - protein, 12 aa, Artificial Sequence
  XTEN Motif Family BC
GSEPATSGTEPS

SEQ ID NO: 73 - protein, 12 aa, Artificial Sequence
  XTEN Motif Family BC
GTSEPSTSEPGA

SEQ ID NO: 74 - protein, 12 aa, Artificial Sequence
  XTEN Motif Family BC
GTSTEPSEPGSA

SEQ ID NO: 75 - protein, 12 aa, Artificial Sequence
  XTEN Motif Family BD
GSTAGSETSTEA

SEQ ID NO: 76 - protein, 12 aa, Artificial Sequence
  XTEN Motif Family BD
GSETATSGSETA

SEQ ID NO: 77 - protein, 12 aa, Artificial Sequence
  XTEN Motif Family BD
GTSESATSESGA

SEQ ID NO: 78 - protein, 12 aa, Artificial Sequence
  XTEN Motif Family BD
GTSTEASEGSAS

SEQ ID NO: 79 - DNA, 4974 bp, Artificial Sequence
  VWF057 (VWF D′D3-Fc with LVPR thrombin site in the linker)
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgatatcgggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcggcggtggaggatccggtggcgggggatccggtggcgggggatccggtggcgggggatccggtggcgggggatccggtggcgggggatccctggtcccccggggcagcggaggcgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 80 - protein, 1662 aa, Artificial Sequence
  VWF057 (VWF D′D3-Fc with LVPR thrombin site linker)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPISGAPTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSGGGGSGGGGSGGGGSGGGGSGGGGSGGGGSGGGGSLVPRGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 4959 bp, Artificial Sequence
  VWF059 (VWF D′D3-Fc with acidic region thombin site in the linker)
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgatatcgggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgatatctgacaagaacactggtgattattacgaggacagttatgaagatatttcagcatacttgctgagtaaaaacaatgccattgaaccaagaagcttctctgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 82 - protein, 1652 aa, Artificial Sequence
  VWF059 (VWF D′D3-Fc with LVPR thrombin site in the linker)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPISGAPTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASISDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 4860 bp, Artificial Sequence
  VWF062 (VWF D′D3-Fc with no thrombin site in the linker)
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgatatcgggcgcgccaacatcagagagcgccacccctgaaagtggtcccgggagcgagccagccacatctgggtcggaaacgccaggcacaagtgagtctgcaactcccgagtccggacctggctccgagcctgccactagcggctccgagactccgggaacttccgagagcgctacaccagaaagcggacccggaaccagtaccgaacctagcgagggctctgctccgggcagcccagccggctctcctacatccacggaggagggcacttccgaatccgccaccccggagtcagggccaggatctgaacccgctacctcaggcagtgagacgccaggaacgagcgagtccgctacaccggagagtgggccagggagccctgctggatctcctacgtccactgaggaagggtcaccagcgggctcgcccaccagcactgaagaaggtgcctcgagcgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 84 - protein, 1619 aa, Artificial Sequence
  VWF062 (VWF D′D3-Fc with no thrombin site in the linker)
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPISGAPTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGASSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 6033 bp, Artificial Sequence
  FVIII 286 (FVIII-Fc with additional a2 region in between FVIII and Fc)
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggctagcttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacggcgcgccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagcctcgagcccaccagtcttgaaacgccatcaagctgaaataactcgtactactcttcagtcagatcaagaggaaatcgattatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaagaacactggtgattattacgaggacagttatgaagatatttcagcatacttgctgagtaaaaacaatgccattgaaccaagaagcttctctgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 86 - protein, 1991 aa, Artificial Sequence
  FVIII 286 (FVIII-Fc with additional a2 region in between FVIII and Fc)
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSPPVLKRHQAEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKNTGDYYEDSYEDISAYLLSKNNAIEPRSFSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 5937 bp, Artificial Sequence
  FVIII 169
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggctagcttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctctcaaaacggcgcgccaggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagcctcgagcccaccagtcttgaaacgccatcaagctgaaataactcgtactactcttcagtcagatcaagaggaaatcgattatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctacgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 88 - protein, 1978 aa, Artificial Sequence
  FVIII 169
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNGAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPASSPPVLKRHQAEITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 15 aa, Artificial Sequence
  Gly/Ser linker
GGGGSGGGGSGGGGS

SEQ ID NO: 90 - protein, 20 aa, Artificial Sequence
  Gly/Ser linker
GGGGSGGGGSGGGGSGGGGS

SEQ ID NO: 91 - DNA, 5379 bp, Artificial Sequence
  VWF034
atgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctatgccgggaagacctgcggcctgtgtgggaattacaatggcaaccagggcgacgacttccttaccccctctgggctggcggagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgccgcattctgcgacaccattgctgcctatgcccacgtgtgtgcccagcatggcaaggtggtgacctggaggacggccacattgtgcccccagagctgcgaggagaggaatctccgggagaacgggtatgaggctgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgatatcgggtacctcagagtctgctacccccgagtcagggccaggatcagagccagccacctccgggtctgagacacccgggacttccgagagtgccacccctgagtccggacccgggtccgagcccgccacttccggctccgaaactcccggcacaagcgagagcgctaccccagagtcaggaccaggaacatctacagagccctctgaaggctccgctccagggtccccagccggcagtcccactagcaccgaggagggaacctctgaaagcgccacacccgaatcagggccagggtctgagcctgctaccagcggcagcgagacaccaggcacctctgagtccgccacaccagagtccggacccggatctcccgctgggagccccacctccactgaggagggatctcctgctggctctccaacatctactgaggaaggtacctcaaccgagccatccgagggatcagctcccggcacctcagagtcggcaaccccggagtctggacccggaacttccgaaagtgccacaccagagtccggtcccgggacttcagaatcagcaacacccgagtccggccctgggtctgaacccgccacaagtggtagtgagacaccaggatcagaacctgctacctcagggtcagagacacccggatctccggcaggctcaccaacctccactgaggagggcaccagcacagaaccaagcgagggctccgcacccggaacaagcactgaacccagtgagggttcagcacccggctctgagccggccacaagtggcagtgagacacccggcacttcagagagtgccacccccgagagtggcccaggcactagtaccgagccctctgaaggcagtgcgccagattctggcggtggaggttccggtggcgggggatccggtggcgggggatccggtggcgggggatccggtggcgggggatccctggtcccccggggcagcggaggcgacaaaactcacacatgcccaccgtgcccagctccagaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 92 - protein, 1778 aa, Artificial Sequence
  VWF034
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCAAFCDTIAAYAHVCAQHGKVVTWRTATLCPQSCEERNLRENGYEAEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPISGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPDIGGGGGSGGGGSLVPRGSGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK